(19) United States
(12) Patent Application Publication
(10) Pub. No.: US 2022/0275458 A1
(43) Pub. Date: Sep. 1, 2022

(54) PREDICTION OF CANCER THERAPY EFFICACY

(71) Applicant: UNIVERSITY OF MASSACHUSETTS, Boston, MA (US)

(72) Inventors: Sharon B. Cantor, Worcester, MA (US); Nicholas Panzarino, Worcester, MA (US); Ke Cong, Worcester, MA (US); Matt Bere, Worcester, MA (US); Sumeet Nayak, Worcester, MA (US)

(21) Appl. No.: 17/629,893

(22) PCT Filed: Aug. 3, 2020
(86) PCT No.: PCT/US20/44774

§ 371 Date: Jan. 25, 2022

Related U.S. Application Data
(60) Provisional application No. 62/883,859, filed on Aug. 7, 2019.
(60) Provisional application No. 62/910,904, filed on Oct. 4, 2019.

(51) Int. Cl. C12Q 1/6886; G16B 20/20

(52) U.S. Cl. CPC C12Q 1/6886; G16B 20/20; C12Q 2600/106

(57) ABSTRACT

BRCA1 or BRCA2 (BRCA)-deficient tumor cells have defects in DNA double strand break repair that are thought to underlie the sensitivity to poly(ADP-ribose) polymerase inhibitor (PARPi). Given the recent finding that PARPi initially accelerates DNA replication, it was proposed that high speed DNA replication ultimately causes DNA breaks that drive synthetic lethality in BRCA deficient cells. Here, an alternative hypothesis is disclosed that PARPi sensitivity results from combined replication dysfunction causing a toxic accumulation of replication-associated single-stranded DNA (ssDNA) gaps. Consistent with this interpretation, PARPi treatment induces ssDNA gaps in replication tracts that are increased in BRCA deficient cells and avoided in FANCJ deficient cells that are not sensitive to PARPi. Furthermore, it is demonstrated that gap suppression underlies known and de novo models of PARPi resistance in BRCA deficient cell lines and tumor samples. Collectively, a molecular link between PARPi sensitivity and ssDNA gaps provides a paradigm for understanding synthetic lethal interactions in BRCA cancer.

### FIELD OF THE INVENTION

The present invention is related to the field of cancer therapy. In particular, the invention predicts the efficacy of a particular cancer therapy. The number of single stranded deoxyribonucleotide gaps near a replication fork induced by chemotherapeutic replication stress is correlated with the clinical efficacy of a chemotherapeutic agent. Consequently, a patient biopsy can be used to predict the in vivo chemotherapeutic efficacy before a cancer therapy is clinically administered.

Background

There are several biomarkers that are proposed to predict the response of cancer to chemotherapy. In particular, biomarkers that detect the integrity of DNA repair have been used to predict the response to genotoxic agents such as cisplatin, as well as to targeted therapies such as poly(ADP-ribose) polymerase (PARP). These agents are expected to have especially potent killing activity in cancer cells with defects in DNA repair; for example, in cancers that lack the hereditary breast cancer genes, BRCA1 and BRCA2, which have many critical functions in the repair of damaged DNA.

Accordingly, the overall art-accepted premise is that genotoxic agents kill cancer cells via inducing DNA damage that is less effectively repaired in BRCA deficient cancer cells due to defects in DNA repair as well as in the protection of DNA replication forks, which leads to the accumulation of more DNA breaks. Therefore, if BRCA1, BRCA2 or the DNA repair protein RAD51 are expressed and functional (not mutated) in tumors, this indicates the integrity of DNA repair by the pathway of homologous recombination and fork protection. Thus, this approach suggests that RAD51 positive staining indicates that the chemotherapy will be less effective and RAD51 staining is a biomarker for poor therapy response. Clinical data has suggested that the DNA-repair based prediction of therapeutic success has met with little prognostic value.

What is needed in the art is an alternative approach that does not depend upon the convention DNA repair hypothesis to predict cancer therapy success.

### SUMMARY OF THE INVENTION

The present invention is related to the field of cancer therapy. In particular, the invention predicts the efficacy of a particular cancer therapy. The number of single stranded deoxyribonucleotide gaps near a replication fork induced by chemotherapeutic replication stress is correlated with the clinical efficacy of a chemotherapeutic agent. Consequently, a patient biopsy can be used to predict the in vivo chemotherapeutic efficacy before a cancer therapy is clinically administered.

In one embodiment, the present invention contemplates a method, comprising: a) providing; i) a cancer cell replicating a deoxyribonucleic acid (DNA) sequence; and ii) a probe configured to detect a single stranded DNA (ssDNA) gap within said DNA sequence; b) scoring a quantity of ssDNA gaps within said DNA sequence with said probe; and c) predicting that a chemotherapeutic agent is efficacious against said cancer cell when said quantity of ssDNA gaps is above a threshold number. In one embodiment, the cancer cell is a breast cancer cell. In one embodiment, the cancer cell is an ovarian cancer cell.

In one embodiment, the present invention contemplates a method, comprising: a) providing; i) a cancer cell replicating a deoxyribonucleic acid (DNA) sequence; and ii) a probe configured to detect a single stranded DNA (ssDNA) gap within said DNA sequence; b) scoring a quantity of ssDNA gaps within said DNA sequence with said probe; and c) predicting that said cancer cell is resistant to a chemotherapeutic agent when said quantity of ssDNA gaps is below a threshold number. In one embodiment, the cancer cell is a breast cancer cell. In one embodiment, the cancer cell is an ovarian cancer cell.

In one embodiment, the present invention contemplates a method, comprising: a) providing; i) a patient comprising at least one cancer cell, wherein said at least one cancer cell is replicating a deoxyribonucleic (DNA) sequence; ii) a probe configured to detect a single stranded DNA (ssDNA) gap within said DNA sequence; and iii) a chemotherapeutic agent; b) removing said at least one cancer cell from said patient; c) contacting said at least one cancer cell with said chemotherapeutic agent; d) scoring a quantity of said ssDNA gaps within said DNA sequence with said probe; and e) predicting that said patient is responsive to said chemotherapeutic agent when said quantity of ssDNA gaps is above a threshold number. In one embodiment, the cancer cell is a breast cancer cell. In one embodiment, the cancer cell is an ovarian cancer cell.

In one embodiment, the present invention contemplates a method, comprising: a) providing; i) a patient comprising at least one cancer cell, wherein said at least one cancer cell is replicating a deoxyribonucleic (DNA) sequence; ii) a probe configured to detect a single stranded DNA (ssDNA) gap within said DNA sequence; and iii) a chemotherapeutic agent; b) removing said at least one cancer cell from said patient; c) contacting said at least one cancer cell with said chemotherapeutic agent; d) scoring a quantity of said ssDNA gaps within said DNA sequence with said probe; and e) predicting that said patient is non-responsive to said chemotherapeutic agent when said quantity of ssDNA gaps is below a threshold number. In one embodiment, the cancer cell is a breast cancer cell. In one embodiment, the cancer cell is an ovarian cancer cell.

In one embodiment, the present invention contemplates a method, comprising: a) providing; i) a patient comprising at least one chemoresistant cancer cell, wherein said at least one cancer cell is replicating a deoxyribonucleic (DNA) sequence; and ii) a compound to induce a single stranded DNA (ssDNA) gap within said DNA sequence; b) treating said patient with said compound, wherein said at least one chemoresistant cancer cell is converted into at least one chemosensitive cancer cell. In one embodiment, the cancer cell is a breast cancer cell. In one embodiment, the cancer cell is an ovarian cancer cell.

In one embodiment, the present invention contemplates a method, comprising: a) providing; i) a patient comprising at least one chemoresistant cancer cell, wherein said at least one cancer cell is replicating a deoxyribonucleic (DNA) sequence; and ii) a compound to induce a single stranded DNA (ssDNA) gap within said DNA sequence; and iii) a chemotherapeutic agent; b) treating said patient with said compound, wherein said at least one chemoresistant cancer cell is converted into at least one chemosensitive cancer cell; and c) treating said patient with said chemotherapeutic agent, wherein said chemotherapeutic agent is efficacious against said at least one chemosensitive cancer cell. In one embodiment, the cancer cell is a breast cancer cell. In one embodiment, the cancer cell is an ovarian cancer cell.

In one embodiment, the present invention contemplates a method, comprising: a) providing; i) a patient comprising at least one chemoresistant cancer cell, wherein said at least one cancer cell is replicating a deoxyribonucleic (DNA) sequence; and ii) a compound to inhibit ssDNA gap filling of said DNA sequence; b) treating said patient with said compound, wherein said at least one chemoresistant cancer cell is converted into at least one chemosensitive cancer cell. In one embodiment, the cancer cell is a breast cancer cell. In one embodiment, the cancer cell is an ovarian cancer cell.

In one embodiment, the present invention contemplates a method, comprising: a) providing; i) a patient comprising at least one chemoresistant cancer cell, wherein said at least one cancer cell is replicating a deoxyribonucleic (DNA) sequence; and ii) a compound to inhibit ssDNA gap filling of said DNA sequence; and iii) a chemotherapeutic agent; b) treating said patient with said compound, wherein said at least one chemoresistant cancer cell is converted into at least one chemosensitive cancer cell; and c) treating said patient with said chemotherapeutic agent, wherein said chemotherapeutic agent is efficacious against said at least one chemosensitive cancer cell. In one embodiment, the cancer cell is a breast cancer cell. In one embodiment, the cancer cell is an ovarian cancer cell.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “cancer cell”, as used herein, refers to any biological cell (e.g., a mammalian cell) which has undergone a proliferative dysregulation such that cell growth cannot be naturally regulated or controlled. For example, such proliferative dysregulation may be due to a genetic mutation occurring in genes including, but not limited to, BRCA1, BRCA2 or RAD51.

The term “replicate”, “replication” or “replicating” as used herein, refers to the activity synthesis of a nucleic acid sequence in a living biological cell (e.g., a mammalian cell). Such replication occurs by a combination of replication machinery components including, but not limited to, a replication origin, a replication fork and/or a replication tract.

The term “single stranded deoxyribonucleotide acid (DNA) gap” or “ssDNA gap” as used herein, refers to a single stranded region in a nascent DNA strand usually occurring near a replication fork. Usually, such ssDNA gaps are formed as a result of high rate replication induced by an intracellular stress factor (e.g., a chemical such as hydroxyurea).

The term “ssDNA gap filling” as used herein, refers to a process by which the single stranded DNA is repaired using a DNA polymerase-based reaction such that the ssDNA gap is converted into double stranded DNA. For example, such gap filling may be performed by a translesion system (TLS).

The term “predicting” as used herein, refers to a quantitative method of detecting and measuring (e.g., scoring) biomarkers such that the total score is either above or below a predetermined threshold level. For example, if a quantity of ssDNA gaps in a cancer cell are scored to be above a predetermined threshold level, a chemotherapeutic agent is predicted to be efficacious against the cancer cell. Alternatively, if a quantity of ssDNA gaps in a cancer cell are scored to be below a predetermined threshold level, a chemotherapeutic agent is predicted not to be efficacious against the cancer cell.

The term “threshold number” as used herein, refers to an empirically derived value which separates cancer cells which are sensitive to therapeutic agent from cancer cells which are resistant to therapeutic agents. For example, the quantity of ssDNA gaps in nascent DNA tracts in non-cancer cells provide a threshold number. Alternatively, a threshold number of ssDNA gaps can be determined by performing a receiver operated characteristic area under the curve (ROC AUC) analysis based upon comparing a cancer population with a non-cancer population.

The term “efficacious” as used herein, refers to a clinically effective treatment response following administration of a therapeutic agent.

The term “resistant” as used herein, refers to a clinically ineffective treatment response following administration of a therapeutic agent. The term “chemotherapeutic agent” as used herein, refers to a compound or molecule that inhibits and/or reduces the growth of a cancer cell.

The term “removing” as used herein, refers to the physical separation of a material from its current environment. For example, a cancer cell may be “removed” from a tumor cell by known biopsy techniques.

The term “contacting” as used herein refers to a physical interaction between two different chemical or materials. In some cases, the contacting may induce a response in either one, or both, of the materials.

The term “converted” as used herein refers to a change in physical and/or biochemical characteristics subsequent to a physical interaction between two different chemical or materials. Usually, the converted material can be verified by subsequent testing for specific markers that confirm its identity.

The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis. The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subj ect.

The term “inhibitory compound” as used herein, refers to any compound capable of interacting with (i.e., for example, attaching, binding etc.) to a binding partner under conditions such that the binding partner becomes unresponsive to its natural ligands. Inhibitory compounds may include, but are not limited to, small organic molecules, antibodies, and proteins/peptides.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “portion” when used in reference to a nucleotide sequence refers to fragments of that nucleotide sequence. The fragments may range in size from 5 nucleotide residues to the entire nucleotide sequence minus one nucleic acid residue.

The term “antibody” refers to immunoglobulin evoked in animals by an immunogen (antigen). It is desired that the antibody demonstrates specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells.

The terms “specific binding” or “specifically binding” when used in reference to the interaction of an antibody and a protein or peptide means that the interaction is dependent upon the presence of a particular structure (i.e., for example, an antigenic determinant or epitope) on a protein; in other words an antibody is recognizing and binding to a specific protein structure rather than to proteins in general. For example, if an antibody is specific for epitope “A”, the presence of a protein containing epitope A (or free, unlabeled A) in a reaction containing labeled “A” and the antibody will reduce the amount of labeled A bound to the antibody.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

As used herein, the term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

As used herein, the terms “siRNA” refers to either small interfering RNA, short interfering RNA, or silencing RNA. Generally, siRNA comprises a class of double-stranded RNA molecules, approximately 20-25 nucleotides in length. Most notably, siRNA is involved in RNA interference (RNAi) pathways and/or RNAi-related pathways. wherein the compounds interfere with gene expression.

As used herein, the term “shRNA” refers to any small hairpin RNA or short hairpin RNA. Although it is not necessary to understand the mechanism of an invention, it is believed that any sequence of RNA that makes a tight hairpin turn can be used to silence gene expression via RNA interference. Typically, shRNA uses a vector stably introduced into a cell genome and is constitutively expressed by a compatible promoter. The shRNA hairpin structure may also cleaved into siRNA, which may then become bound to the RNA-induced silencing complex (RISC). This complex binds to and cleaves mRNAs which match the siRNA that is bound to it.

As used herein, the term “microRNA”, “miRNA”, or “pRNA” refers to any single-stranded RNA molecules of approximately 21-23 nucleotides in length, which regulate gene expression. miRNAs may be encoded by genes from whose DNA they are transcribed but miRNAs are not translated into protein (i.e. they are non-coding RNAs). Each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

The term “sample” or “biopsy” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

As used herein, the term “probe” refers; to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

### BRIEF DESCRIPTION OF THE FIGURES

FIG. 1 illustrates single stranded DNA gap prediction of cancer therapy response and the role of replication regulatory genes (e.g., BRCA1, BRCA2, or RAD51) during chemotherapeutic drug —induced replication stress.
Upper Therapy Resistance Panel: Functional BRCA1, BRCA2, or RAD51 slow replication stress rates induced by chemotherapeutic drugs thereby preventing DNA gap formation and cancer cell death.
Therapy Sensitivity Panel: Non-functional BRCA1, BRCA2, or RAD51 permits replication stress rates induced by chemotherapeutic drugs to continue such that DNA gaps accumulate resulting in cancer cell death (BRACAness).
Lower Therapy Resistance Panel: Gaps are avoided because the replication rate either slows/pauses or the replication continues but with gap filling (i.e., translesion synthesis). FIG. 2 illustrates various methods by which ssDNA gaps may be detected in a nucleic acid segment, for example ssDNA gaps that are induced near a replication fork.

FIG. 3 presents exemplary data showing DNA analogue incorporation with non-denaturing immunofluorescence (IF) that shows that BRCA1-deficient cells have more PARPi induced gaps as compared to controls.

FIG. 4 presents exemplary data showing DNA fiber cutting with an S1 nuclease demonstrating that BRCA1-deficient cells have more PARPi-induced gaps.

FIG. 5 presents an illustrative model of BRCA-RAD51 function in fork restraint and mechanisms of gap suppression that confer chemoresistance.

FIG. 6 presents exemplary data showing that TLS reduces replication fork-slowing and -reversal without gap induction.

FIG. 6A: Western blot with the indicated antibodies (Abs) of lysates from U2OS CRISPR control and FANCJ CRISPR knockout (K/O) cells that were complemented with empty vector (V), wild-type (WT) FANCJ and the FANCJ-BRCA1 binding deficient mutant (S990A).

FIG. 6B/C: Schematic of the DNA fiber assay and the quantification of CldU tract length. Each dot represents one fiber; Experiments were performed in biological triplicate with at least 100 fibers per replicate. Red bars represent the median. Statistical analysis according to two-tailed Mann-Whitney test; ****, P<0.0001. Mean and 95% confidence intervals are shown.

FIG. 6D: Quantification of frequency of fork reversal in the U2OS cell lines following indicated treatment. Around 75-90 replication intermediates were analyzed from two independent experiments. EM image and cartoon showing reversed fork structures.

FIG. 6E: Same as FIG. 6C.

FIG. 7 presents exemplary data showing that TLS disrupts global replication stress response without ssDNA gap induction.

FIG. 7A: Schematic of EdU labeling;

FIG. 7B: CldU labelling and representative immunofluorescence images of the U2OS cell lines with or without the ATR inhibitor (ATRi) stained as indicated for EdU using the ClickiT chemistry or CldU Ab to detect ssDNA and DAPI. At least 300 cells were counted from multiple fields and graphed.

FIG. 8 presents exemplary data showing; i) TLS factor targeting; ii) REV1 restoration of replication rate slowing; and iii) ssDNA gaps and the cisplatin sensitivity.

FIG. 8A: Schematic and quantification of CldU tract length in the indicated U2OS cells.

FIG. 8B: Representative images and quantification of EdU positive cells in the presence or absence of HU with or without TLSi 1.

FIG. 8C: Schematic of a combined assay and representative images of the FANCJS990A U2OS cells with indicated staining. Statistics and data collection as described in FIG. 6 and FIG. 7.

FIG. 8D: Cell survival assays as indicated. Data represent the mean percent±SD of survival from three independent experiments.

FIG. 9 presents exemplary data showing that TLSi 1 disrupts DNA replication, induces ssDNA and reduces clonogenic capacity of TLS cancer cells.

FIGS. 9A-C: Quantification of EdU or ssDNA positive cells with the indicated treatments.

FIG. 9D: Representative clonogenic assays with the indicated cell lines and treatments.

FIG. 10 presents exemplary data showing changes in the composition of the TLS replisome using a Western blot with the indicated Abs and U2OS cell lines.

FIG. 11 presents exemplary data showing that TLS induction by p21 depletion is inhibited by the TLSi.

FIG. 11A: Western blot with indicated Abs and shRNA reagents in U2OS cells.

FIG. 11B: Combined assay schematic and quantification.

FIG. 11C: Representative photomicrographs.

FIG. 12 presents exemplary data showing that the FANCJ gene is required for HeLa cell replication and clonogenic capacity.

FIG. 12A: Western blot with indicated cell lines and Abs.

FIG. 12B: Quantification of EdU positive cells in the indicated cells with or without HU.

FIG. 12C: Representative clonogenic assays with indicated cell lines.

FIG. 13 presents exemplary data showing that high REV1 mRNA predicts poor cancer therapy response in BRCA2 deficient ovarian cancer. TCGA ovarian cancer patients with germline mutations that deactivate BRCA2 (e.g. truncated by premature stop codon) were identified using the Gnome Analysis Tool Kit, and plotted survival in cBioPortal for high and low REV1 mRNA levels (top 25% vs bottom 25%).

FIG. 14 presents exemplary embodiments of several potential lead compounds as inhibitors of REV1.

FIG. 15 presents exemplary data showing TLS vs non-TLS cells that will be used to test TLS inhibitors by imaging.

FIG. 15A: Schematic and representative images showing expected response of distinct cells to indicated treatments.

FIG. 15B: Outline and model of screen to identify TLSi that are selective to TLS cancer cells.

FIG. 16 presents an illustrative flow chart showing a method for a combination assay of TLS inhibition and cologenesis.

FIG. 17 presents exemplary azo group analogues for REV1 inhibitor compound 2.

FIG. 18 presents an illustrative representation of how cancer might subvert replication stress using TLS that provides a target for global cancer therapy.

FIG. 19 presents exemplary data showing that BRCA2 mediates replication fork restraint and ssDNA gap avoidance:

FIG. 19A: Western blot and cell survival assay.

FIG. 19B: Schematic and quantification of CldU tract lengths. Experiments were performed in biological triplicate with at least 100 fibers per replicate. Statistical analysis according to two-tailed Mann-Whitney test; *** p<0.001. Mean and 95% confidence intervals are shown.

FIG. 19C: Schematic, representative images and quantification of nuclear imaging, p<0.05 (*) as determined by t-test of biological triplicate experiments.

FIG. 19D: Nondenaturing fiber assay identifies exposed ssDNA in newly replicating regions after stress in PEO1, but not C4-2 cells. Regions of active replication were detected with EdU-ClickIT (green signal). p<0.01 (***) as determined by t-test of biological triplicate experiments.

FIG. 19E: Data interpretation. Circle shows region at fork typically analyzed for degradation.

FIG. 20 presents exemplary data showing that CHD4 deletion reduces ssDNA gaps and cisplatin sensitivity but replication forks are more unrestrained:

FIG. 20A: Western blot and cell survival assay.

FIG. 20B: Schematic and quantification of CldU track length in restraint S1 cutting, and fork degradation.

FIG. 20C: Schematic and quantification of nuclear imaging.

FIG. 20D: A non-denaturing fiber assay.

FIG. 20E: Data interpretation: p<0.05 (*), p<0.01 (**)

FIG. 21 presents exemplary data showing that SILAC proteomics identifies proteins required for cisplatin sensitivity in BRCA2 deficient cancers.

FIG. 21A: Experimental outline.

FIG. 21B: Proteomics reveals proteins significantly enriched in a in BRCA2 mutant (red) and proficient (blue) cells 18 h after cisplatin treatment. Black: not significant.

FIG. 21C: PEO1 cells with the noted shRNAs were analyzed in survival assays following cisplatin.

FIG. 21D: Reduced ZFHX3 mRNA levels predict poor patient response to therapy (progression free survival) for BRCA2 deficient ovarian cancers from the TCGA database.

FIG. 22 presents exemplary data showing that suppression of ssDNA gaps predicts cisplatin resistance in cell culture and patient xenografts.

FIG. 22A: Schematic and quantification of CldU track lengths in restraint and S1 cutting assay in indicated cells.

FIG. 22B: Data interpretation.

FIG. 22C: PEO1 xenograft and BRCA1 deficient patient derived xenografts.

FIG. 23 presents exemplary data showing that inactivation of MRE11 or fork reversal does not suppress ssDNA gaps or cisplatin sensitivity.

FIG. 23A: Western blot and survival assay

FIG. 23B: Schematic and quantification of CldU track lengths in fork protection assay

FIG. 23C: Restraint assay

FIG. 23D: S1 cutting assay.

FIG. 24 presents exemplary data showing that RAD51 mutant cells have restraint defect and gaps. Schematic and quantification of CldU track lengths in restraint S1 assay.

FIG. 25 presents exemplary data of a combined assay to survey global replication and gap induction. Schematic of the combined assay and representative images of BRCA-RAD51 (C4-2) proficient or deficient (PEO1 w or w/o CHD4 depletion). Cells with indicated staining and treatment with HU.

FIG. 26 presents exemplary data showing that PARPi-induced gaps are exacerbated in BRCA K/O cells and avoided in FANCJ K/O cells.

FIG. 26A: Immunoblot assay.

FIG. 26B: Cell survival assay.

FIG. 26C: Schematic and quantification of CldU track length in fork restraint,

FIG. 26D: S1 cutting assay.

FIG. 26E: Non-denaturing IF detection of ssDNA.

FIG. 26F: Data interpretation.

FIG. 27 illustrates one embodiment of a Venn diagram to identify the protein machinery of ssDNA gap formation versus ssDNA gap avoidance.

FIG. 28 presents exemplary data showing that the suppression of ssDNA gaps predicts PARPi resistance in patient xenografts. Schematic and quantification of CldU track lengths in S1 cutting assay in BRCA1 deficient patient derived xenografts.

FIG. 29 presents exemplary data showing that TLS promotes replication during stress and suppresses ssDNA gaps.

FIG. 29A: Western blot analysis with the indicated Abs of WCE from U2OS CRISPR control and FANCJ CRISPR knockout (K/O) cells and FANCJ K/O cells complemented with empty vector (V), wild-type (WT) FANCJ and the FANCJ-BRCA1 binding deficient mutant (S990A).

FIG. 29B: Experimental schematic, representative immunofluorescence images and quantification of EdU and ssDNA positive cells. Cells were stained for EdU using the ClickiT chemistry and for ssDNA using CldU specific Ab under non-denaturing conditions and DAPI. Percent EdU and ssDNA positive cells were quantitated from around 300 cells counted from multiple fields.

FIG. 29C: Experimental schematic and representative immunofluorescence images at 100X following label with EdU in presence or absence of HU and/or TLSi and stained for EdU using the ClickiT chemistry and stained for ssDNA using ssDNA specific antibody and DAPI.

FIG. 29D: Western blot analysis with the indicated Abs of whole cell extract (WCE) from U2OS cells expressing shRNA against NSC or p21.

FIGS. 29E & 29F: Experimental schematic and quantification of EdU and ssDNA positive cells. Cells were stained as described above.

FIG. 30 presents exemplary data showing:

FIG. 30A: Cell survival assays with U2OS FANCJ K/O complemented cells (complemented with FANCJWT (WT), empty vector (V) and the FANCJ-BRCA1 binding deficient, FANCJS990A (pro-TLS) treated with increasing concentrations of MMC.

FIGS. 30B & 30C: Experimental schematic, representative images and quantification of the EdU positive cells. Staining and quantitation performed as described in FIG. 29.

FIG. 30E: Cell survival assays with U2OS FANCJ K/O complemented cells under increasing concentrations of either cisplatin alone or TLSi alone or cisplatin plus TLSi. Data represent the mean percent±s.d. of survival from three independent experiments

FIG. 31 presents exemplary data showing:

FIG. 31A: Western blot analysis with the indicated Abs of WCE from U2OS cells expressing shRNA against NSC or p21, partly shown in FIG. 29D.

FIG. 31B: Experimental schematic and representative immunofluorescence images following label with EdU in presence or absence of varying dose of HU. Cells stained for EdU using the ClickiT chemistry and DAPI.

FIGS. 31C & 31D: Experimental schematic, quantification and representative images of EdU and ssDNA positive cells. Staining and quantitation performed as described in FIG. 29.

FIG. 32 presents exemplary data showing that TLS limits replication fork slowing, reversal, degradation and gap induction. Experiments were performed in complemented U2OS FANCJ K/O cells. For every DNA fiber assay analysis, each dot represents one fiber. Experiments were performed in biological triplicate with at least 100 fibers per replicate. Red bars represent the median. Statistical analysis according to two-tailed Mann-Whitney test; ****, p<0.0001. Mean and 95% confidence intervals are shown

FIG. 32A: Quantification of the frequency of fork reversal following treatment with 4 mM HU for 2 h. Following number of replication intermediates were analyzed; untreated sample—94 for (Control) and 84 for (pro-TLS), while for HU treated—130 for (Control) and 176 for (pro-TLS) from two independent experiments.

FIG. 32B: Experimental schematic and quantification of CldU tract length.

FIG. 32C: Experimental schematic and quantification of CldU/IdU ratio.

FIGS. 32D & 32E: Experimental schematic and quantification of CldU tract length after co-incubation with 0.5 mM HU or 2J/m2 of UV or along with TLSi.

FIG. 32F: Experimental schematic and quantification of CldU track length following −/+S1 .

FIG. 33 presents exemplary data showing:

FIG. 33A: Western blot analysis with the indicated Abs of lysates from FA-J cells complemented with FANCJWT (WT), empty vector (V) and the FANCJ-BRCA1 binding deficient, FANCJS990A (pro-TLS), the FANCJ helicase dead mutant (K52R) and the FANCJ-BRCA1 binding deficient and helicase dead (K52R) double mutant.

FIG. 33B: Cell survival assays with FA-J complemented cells under increasing concentrations of cisplatin. Data represent the mean percent±SD of survival from three independent experiments. All the DNA fiber assay experiments were performed in the complemented FA-J cells.

FIG. 33C: Experimental schematic and the quantification of CldU tract length.

FIG. 33D: Experimental schematic and quantification of CldU/IdU ratio.

FIG. 33E: Experimental schematic and the quantification of CldU tract length after a long term degradation.

FIG. 33F: Experimental schematic and quantification of CldU tract length after co-incubation with 0.5 mM HU or 2J/m2 of UV.

FIG. 33G: Experimental schematic and quantification of CldU track length following −/+S1 nuclease.

For every DNA fiber assay analysis (C-G), each dot represents one fiber. Experiments were performed in biological triplicate with at least 100 fibers per replicate. Red bars represent the median. Statistical analysis according to two-tailed Mann-Whitney test; ****, p<0.0001. Mean and 95% confidence intervals are shown.

FIG. 34 presents exemplary data showing:

FIG. 34A: Western blot analysis with the indicated Abs of WCE from PEO1 cells expressing empty vector (V), FANCJWT (WT), and the FANCJ-BRCA1 binding deficient, FANCJS990A (pro-TLS). Cell survival assays with above three cell lines under increasing concentrations of cisplatin. Data represent the Mean percent±s.d. of survival from three independent experiments. Experimental schematic and quantification of CldU/IdU ratio and CldU tract length.

FIG. 34B: Experimental schematic and quantification of CldU tract length, percent CldU tract with IdU, percent only IdU and percent only CldU tract in FA-J complemented cells.

For every DNA fiber assay analysis, each dot represents one fiber. Experiments were performed in biological triplicate with at least 100 fibers per replicate. Red bars represent the median. Statistical analysis according to two-tailed Mann-Whitney test; ****, p<0.0001. Mean and 95% confidence intervals are shown.

FIG. 35 presents exemplary data that;

FIG. 35A: Western blot analysis with the indicated Abs of WCE from HeLa CRISPR control and FANCJ CRISPR knockout (K/O) cells.

FIG. 35B: Cell survival assays with HeLa CRISPR control and FANCJ CRISPR knockout (1(10) cells under increasing concentrations of cisplatin. Data represent the mean percent±SD of survival from three independent experiments.

FIG. 35C: Experimental schematic and quantification of EdU positive cells. Cells were stained for EdU using the ClickiT chemistry and DAPI. Percent EdU positive cells were quantitated from around 300 cells counted from multiple fields.

FIG. 35D: Representative images and quantification of the colony formation assay.

FIG. 35E: Western blot analysis with the indicated Abs of whole cell extract (WCE) from HeLa FANCJ K/O cells expressing shRNA against NSC or p21.

FIG. 35F: Experimental schematic and quantification of EdU and ssDNA positive cells performed as described in FIG. 29.

FIG. 35G: Representative images and quantification of the colony formation assay with and without continuous presence of TLSi (20 μM).

FIG. 36 presents exemplary data that TLS subverts the replication stress response to promote cancer fitness.

FIG. 36A: Experimental schematic and quantification of EdU and ssDNA positive cells. Cells were stained for EdU using the ClickiT chemistry and for ssDNA using CldU specific Ab under non-denaturing conditions and DAPI. Percent EdU and ssDNA positive cells were quantitated from around 300 cells counted from multiple fields.

FIGS. 36B & C: Representative images and quantification of the colony formation assay with and without continuous presence of TLSi (20 μM).

FIG. 36D: Model summarizing that TLS is a replication stress avoidance mechanism in cancer.

FIG. 37 presents exemplary data that:

FIG. 37(A): Experimental schematic and quantification of EdU positive cells. Cells were stained for EdU using the ClickiT chemistry and DAPI. Percent EdU positive cells were quantitated from around 300 cells counted from multiple fields.

FIG. 37(B): Experimental schematic and representative immunofluorescence images of EdU and ssDNA positive cells. Cells were stained for EdU using the ClickiT chemistry and for ssDNA using CldU specific Ab under non-denaturing conditions and DAPI, after co-incubation with 0.5 mM HU and or with TLSi.

FIGS. 37C-D: Experimental schematic and quantification of EdU and ssDNA positive cells. Cells were stained for EdU using the ClickiT chemistry and for ssDNA using CldU specific Ab under non-denaturing conditions and DAPI. Percent EdU and ssDNA positive cells were quantitated from around 300 cells counted from multiple fields.

FIG. 37E: Representative images of the colony formation assay with and without continuous presence of TLSi (20 μM).

FIG. 38 presents exemplary data of:

FIG. 38A: Western blot analysis with the indicated antibodies of lysates from Control, FANCJ K/O and BRCA1 K/O RPE1 cells. WCE, whole cell lysates.

FIG. 38B: Cell survival assays for Control, FANCJ K/O and BRCA1 K/O RPE1 cells under increasing concentrations of olaparib or cisplatin. Data represent the mean percent±SD of survival for each dot.

FIG. 38C: Schematic, representative images, and quantification of the length of dual-color tracts in indicated cells following olaparib treatment (10 μM, 2 hours).

FIG. 38D: Schematic, representative images, and quantification of mean ssDNA intensity for indicated cells following CldU pre-labeling and olaparib release (10μM, 2 hours). At least 300 cells are quantified from three independent experiments. Bars represent the mean±SD. Statistical analysis according to t test. Scale bars, 50 μm.

FIG. 38E: Schematic and quantification of the length of IdU tracts with or without S1 nuclease incubation in indicated cells following olaparib treatment (10 μm, 2 hours). For (C) and (E), each dot represents one fiber; at least 200 fibers are quantified from two independent experiments. Red bars represent the median. Statistical analysis according to two tailed Mann-Whitney test.

FIG. 38F: Western blot analysis with the indicated antibodies of lysates from U2OS cells expressing siRNA against non-silencing control, FANCJ and BRCA1.

FIG. 38G: Quantification of average RPA density at each fork in STORM analysis for indicated cells and treatment. Bars represent the mean±SD. Each dot represents the pair localized RPA density from one nucleus. Statistical analysis according to t test. All p values are described in Statistical methods.

FIG. 39 presents exemplary data of:

FIG. 39A: Quantification of the ratio of IdU/CldU in indicated cells following olaparib treatment (10 μM, 2 hours). Each dot represents one fiber; at least 200 fibers are quantified from two independent experiments. Red bars represent the median. Statistical analysis according to two tailed Mann-Whitney test.

FIG. 39B: Experimental scheme for STORM imaging of replication forks and representative super-resolution image of a single U2OS nucleus labeled for nascent DNA (using EdU, red), RPA70 (grey), MCM6 (green) and PCNA (blue). Scale bar, 3000nm.

FIG. 39C: Cell survival assays for Control, FANCJ K/O and BRCA1 K/O RPE1 cells under increasing concentrations of camptothecin (CPT). Data represent the mean percent±SD of survival for each dot.

FIGS. 39(D and 39E: Western blot analysis with the indicated antibodies of lysates from Control, FANCJ K/O U2OS cells (C) and 293T cells (D). Cell survival assays for indicated cells under increasing concentrations of olaparib and mitomycin C (MMC). Data represent the mean percent±SD of survival for each dot.

FIG. 40 presents exemplary data of:

FIGS. 40A and 40B: Western blot analysis with the indicated antibodies of lysates from U20S(A) and RPE1(B) cells expressing shRNA against non-silencing control (NSC), p21(A) and p21(B).

FIGS. 40C and 40D: Schematic and quantification of the length of CldU tracts in indicated U2OS and RPElcells following olaparib treatment (10 μM, 2 hours). For (C) and (D), each dot represents one fiber; at least 100 fibers are quantified. Red bars represent the median. Statistical analysis according to two tailed Mann-Whitney test.

FIGS. 40E and 40F: Cell survival assays for indicated U2OS and RPE1 cells under increasing concentrations of olaparib. Data represent the mean percent±SD of survival for each dot.

FIGS. 40G; and 40H: Schematic and quantification of mean ssDNA intensity for indicated cells following CldU pre-labeling and olaparib release (10 μm, 2 hours). At least 200 cells are quantified from two independent experiments. Bars represent the mean±SD. Statistical analysis according to t test. All p values are described in Statistical methods.

FIG. 41 presents exemplary data of:

FIG. 41A: Cell survival assays for T2, BR5 and BR5-R1 (BR5-derived resistant cells) cells under increasing concentrations of olaparib without or with ATR inhibitor (VE-821, 1 μM). Data represent the mean percent±SD of survival for each dot.

FIG. 41B: Schematic and quantification of the length of IdU tracts in indicated cells following olaparib treatment (10 μM, 2 hours). Each dot represents one fiber; at least 200 fibers are quantified from two independent experiments. Red bars represent the median. Statistical analysis according to two tailed Mann-Whitney test.

FIG. 41C: Schematic and quantification of mean ssDNA intensity for BR5 and BR5-R1 cells following CldU pre-labeling and olaparib release (10 μM, 2 hours), without or with ATR inhibitor (VE-821, 1 μM). At least 200 cells are quantified from two independent experiments. Bars represent the mean±SD. Statistical analysis according to t test.

FIG. 41D: Schematic and quantification of the length of IdU tracts with or without S1 nuclease incubation in indicated PDX samples and olaparib treatment (10 μm, 2 hours). Each dot represents one fiber; at least 150 fibers are quantified. Red bars represent the median. Statistical analysis according to two tailed Mann-Whitney test. All p values are described in Statistical methods.

FIG. 42 presents exemplary data of:

FIG. 42A: Cell survival assays for T2, BR5 and BR5-R1 cells under increasing concentrations of olaparib with ATR inhibitor (VE-821, 1 μM) and ATR inhibitor alone. Data represent the mean percent±SD of survival for each dot.

FIG. 42B: Cell survival assays for C4-2 and PEO1 cells under increasing concentrations of Olaparib. Data represent the mean percent±SD of survival for each dot.

FIG. 42C: Schematic and quantification of the length of IdU tracts in PEO1 and C4-2 cells following olaparib treatment (10 μM, 2 hours). Each dot represents one fiber, at least 100 fibers are quantified. Red bars represent the median. Statistical analysis according to two tailed Mann-Whitney test.

FIG. 42D: Schematic and quantification of mean ssDNA intensity for indicated cell lines following CldU pre-labeling and olaparib release (1 μM, 4 hours). At least 200 cells are quantified from two independent experiments. Bars represent the mean±SD. Statistical analysis according to t test. All p values are described in Statistical methods.

FIG. 43 presents exemplary data of:

FIG. 43A: Western blot analysis with the indicated antibodies of lysates from T2 and BR5 cells expressing shRNA against NSC, 53BP1(A) and 53BP1(B). FIG. 43B: Cell survival assays for indicated cells under increasing concentrations of olaparib. Data represent the mean percent±SD of survival for each dot.

FIG. 43C: Quantification of mean ssDNA intensity for BR5 cells expressing shRNA against NSC, 53BP1(A) and 53BP1(B) following CldU pre-labeling and olaparib release (10 μM, 2 hours).

FIG. 44 presents exemplary data of:

FIG. 44A: A schematic posing the question of whether ssDNA gaps could sensitize cells despite HR or FP proficiency?

FIG. 44B: Cell survival assays for patient fibroblasts (RA2630) RAD51 T131P and RAD51 CRISPR corrected cells under increasing concentrations of olaparib. Data represent the mean percent±SD of survival for each dot.

FIG. 44C: Schematic and quantification of mean ssDNA intensity for indicated cell lines following CldU prelabeling and olaparib release (10 μM, 2 h). The gap ratio is the fold change of ssDNA intensity from treated to untreated.

FIG. 44D: Western blot analysis with the indicated antibodies of lysates from patient fibroblasts (RA2630) RAD51 T131P cells expressing shRNA against non-silencing control (NSC) and RADX.

FIG. 44E: Cell survival assays for patient fibroblasts (RA2630) RAD51 T131P expressing shRNA against nonsilencing control (NSC) and RADX under increasing concentrations of olaparib. Data represent the mean percent±SD of survival for each dot.

FIG. 44F: Quantification of mean ssDNA intensity for indicated cell lines following CldU pre-labeling and olaparib release (10 μM, 2 hours). For FIG. 44C and FIG. 44F, at least 200 cells are quantified from two independent experiments. Bars represent the mean±SD. Statistical analysis according to t test. All p values are described in Statistical methods. The gap ratio is the fold change of ssDNA intensity from treated to untreated.

FIG. 44G: Model summarizing that gaps are the determent of PARPi therapy response.

FIG. 45 presents exemplary data of Analysis of rescue of BRCA-RAD51 deficiency.

FIG. 45A: Western blot analysis with the indicated antibodies of lysates from T2 and BR5 cells expressing shRNA against NSC, 53BP1(A) and 53BP1(B).

FIG. 45B: Cell survival assays for indicated cells under increasing concentrations of olaparib. Data represent the mean percent±SD of survival for each dot.

FIG. 45C: Quantification of mean ssDNA intensity for BR5 cells expressing shRNA against NSC, 53BP1(A) and 53BP1(B) following CldU pre-labeling and olaparib release (10 μM, 2 hours). At least 200 cells are quantified from two independent experiments. Bars represent the mean±SD. Statistical analysis according to t test.

FIG. 46 presents exemplary data of a quantification of the CldU/IdU ratio after 3 mM HU treatment for 5 h in patient fibroblasts (RA2630) RAD51 T131P expressing shRNA against non-silencing control (NSC) and RADX. Each dot represents one fiber. For each analysis, at least 100 fibers are quantified. Red bars represent the median. Statistical analysis according to two-tailed Mann-Whitney test. All p values are described in Statistical methods

FIG. 47 presents exemplary data showing that BRCA2 deficient cancer cells fail to restrain replication in the presence of stress, generating regions of ssDNA gaps that are destroyed after continued exposure.

FIG. 47A: Left, Western blot detects truncated BRCA2 protein in BRCA2 deficient PEO1 cells, and detects full-length BRCA2 protein in BRCA2 proficient C4-2 cells that are derived from PEO1 cells. Right, cell survival assay confirms PEO1 cells are hypersensitive to cisplatin compared to C4-2 cells.

FIG. 47B: Schematic and quantification of CldU track length shows (white panel) that PEO1 cells fail to arrest replication in the presence of stress. These regions are degraded by S1 nuclease (light grey panel), and are also destroyed after continued exposure to replication stress (dark grey panel). Each dot represents one fiber. Experiments were performed in biological triplicate with at least 100 fibers per replicate. Statistical analysis according to two-tailed Mann-Whitney test; *** p<0.001. Mean and 95% confidence intervals are shown.

FIG. 47C: Schematic, representative images and quantification of nuclear imaging identifies a greater percentage of EdU positive cells in PEO1 as compared to C4-2. p<0.05 (*) as determined by t-test of biological triplicate experiments.

FIG. 47D: Nondenaturing fiber assay identifies exposed ssDNA in newly replicating regions after stress in PEO1, but not C4-2 cells. Regions of active replication were detected with EdU-ClickIT (green signal). p<0.01 (***) as determined by t-test of biological triplicate experiments.

FIG. 47E: Model of fiber assay interpretation.

FIG. 48 presents exemplary data showing that BRCA deficient cancer cells fail to restrain replication in the presence of stress and gaps develop.

FIG. 48A: Western blot confirms BRCA deficiency and complementation in BRCA2 deficient VC-8 hamster cells. and

FIG. 48B Western blot confirms BRCA deficiency and complementation in BRCA1 deficient breast cancer cells HCC1937.

FIG. 48C: Western blot confirms BRCA deficiency and complementation in BRCA1 deficient breast cancer cells MDA-MB-436. Schematic (above) and quantification of CldU track length shows BRCA complementation restores fork restraint.

FIG. 48D: Schematic, representative images, and quantification of CldU track length shows that PEO1 and C4-2 have similar track lengths in untreated conditions.

FIG. 48E: Same as D, but with a 2 h pulse.

FIG. 48F: Schematic and representative fiber images for FIG. 1B show that PEO1 cells fail to arrest replication in the presence of stress.

FIG. 48G: Schematic, representative images and quantification of nuclear imaging identifies a similar percentage of EdU positive cells in PEO1 as compared to C4-2 in unchallenged conditions.

FIG. 48H: Schematic and quantification of CldU track length shows BRCA1 deficient HCC1937 (top) and MDA-MB-436 (bottom) are sensitive to S1 nuclease. Each dot represents one fiber. Experiments were performed in biological duplicate with at least 100 fibers per replicate. Statistical analysis according to two-tailed Mann-Whitney test; *** p<0.001. Mean and 95% confidence intervals are shown.

FIG. 49 presents exemplary data showing that CHD4 depletion suppresses ssDNA gaps but does not restore fork restraint.

FIG. 49A: Left, Western blot confirms CHD4 is depleted by shRNA compared to non-silencing control (NSC) in BRCA2 deficient PEO1. Right, cell survival assay confirms PEO1 with depleted CHD4 are resistant to cisplatin compared to PEO1 NSC.

FIG. 49B: Schematic and quantification of CldU track length shows (white panel) that PEO1 with depleted CHD4 increase replication in the presence of stress (white panel). These regions are protected from S1 nuclease (light grey panel), and are also protected after continued exposure to replication stress (dark grey panel). Each dot represents one fiber. Experiments were performed in biological triplicate with at least 100 fibers per replicate. Statistical analysis according to two-tailed Mann-Whitney test; *** p<0.001. Mean and 95% confidence intervals are shown.

FIG. 49C: Schematic, representative images and quantification of nuclear imaging identifies a greater percentage of EdU positive cells in CHD4 depleted PEO1 as compared to NSC. p<0.01 (**) as determined by t-test of biological triplicate experiments.

FIG. 49D: Nondenaturing fiber assay identifies that ssDNA in newly replicating regions after stress is reduced when CHD4 is depleted in PEO1 cells. Regions of active replication were detected with EdU-ClickIT (green signal). p<0.05 (*) as determined by t-test of biological duplicate experiments.

FIG. 49E: Model of fiber assay interpretation.

FIG. 50 presents exemplary data showing a replication restraint controls and depletion analysis.

FIG. 50A: Western blot confirms CHD4 is depleted by shRNA (61 and 62) compared to non-silencing control (NSC) in BRCA2 deficient PEO1.

FIG. 50B: Schematic and representative images of CIdU track length shows that CHD4 depletion increases replication in the presence of stress.

FIG. 50C: Schematic and representative images of nuclear imaging show that CHD4 shRNA and NSC have similar numbers of EdU positive cells in unchallenged conditions.

FIG. 50D: Schematic and representative images show that CHD4 shRNA and NSC have similar track lengths in unchallenged conditions with 30min labeling.

FIG. 50E: Schematic and representative images show that CHD4 shRNA and NSC have similar track lengths in unchallenged conditions with 2 h labeling.

FIG. 50F: Western blot of shRNA confirms FEN1 is depleted by shRNA (31 and 32) compared to non-silencing control (NSC) in BRCA2 deficient PEO1.

FIG. 50G: Cell survival assay shows PEO1 cells with inhibited MRE11 or depleted SMARCAL1 are not resistant to cisplatin as compared to controls.

FIG. 50H: Schematic and quantification of CIdU track length in MDA-MB-436 xenografts shows that complementation with BRCA1 protects from S1 .

FIG. 51 presents exemplary data showing that suppression of ssDNA gaps accurately predicts poor therapy response in both cell culture and patient xenografts.

FIG. 51A: Overview of the SILAC CHD4 immunoprecipitation experiment.

FIG. 51B: SILAC immunoprecipitation reveals that CHD4 interacts with ZFHX3, FEN1, and EZH2 after cisplatin treatment. Red and blue circles are proteins significantly enriched in the CHD4 network of either PEO1 or C4-2 cells. Yellow circles are known CHD4 interacting partners(O'Shaughnessy and Hendrich, 2013). Black plus signs represent proteins not significantly enriched in the CHD4 network of either PEO1 or C4-2. Three biological replicates were performed; see methods section for statistical analysis.

FIG. 51C: Western blot confirms ZFHX3 is depleted by shRNA in PEO1 as compared to NSC. Cell survival assay confirms PEO1 with depleted ZFHX3 are resistant to cisplatin compared to PEO1 NSC.

FIG. 51D: Reduced ZFHX3 mRNA levels predict poor patient response to therapy (progression free survival) for BRCA2 deficient ovarian cancers from the TCGA database.

FIG. 51E:) Schematic and quantification of CldU track length shows that depletion of CHD4 (shRNA 61), ZFHX3 or FEN1, or inhibition of EZH2, increase replication in the presence of stress (white panel) and protect nascent DNA from S1 nuclease (gray panel).

FIG. 51F: Schematic and quantification of CldU track length shows S1 fiber sensitivity is suppressed in BRCA1 deficient patient derived xenografts that have acquired chemoresistance. Each dot represents one fiber. Experiments were performed in biological triplicate with at least 100 fibers per replicate; the xenograft fiber assay was performed in duplicate. Statistical analysis according to two-tailed Mann-Whitney test; *** p<0.001. Mean and 95% confidence intervals are shown.

FIG. 52 presents exemplary data shwoing that ssDNA replication gaps, and not fork protection or homologous recombination, determine patient response to chemotherapy and define BRCAness.

FIG. 52A: Schematic and quantification of CldU track length in PEO1 cells shows that depleted SMARCAL1 or inhibited MRE11 does not increase replication in the presence of stress,

FIG. 52B: Schematic and quantification of CldU track length in PEO1 cells shows that depleted SMARCAL1 or inhibited MRElldoes not protect from S1 nuclease as found for CHD4 depletion.

FIG. 52C: Neither SMARCAL1 nor MRE11 mRNA levels predict response of BRCA2 deficient ovarian cancer patients in TCGA dataset. In contrast, CHD4 mRNA levels do predict response in these patients.

FIG. 52D: Schematic and quantification of CldU track length shows (white panel) that fibroblasts from a Fanconi Anemia-like patient with a mutant allele of RAD51 (T131P; homologous recombination proficient cells, cisplatin sensitive) fail to arrest replication in the presence of stress, and these regions are degraded by S1 nuclease (light grey panel). Each dot represents one fiber. Experiments were performed in biological triplicate with at least 100 fibers per replicate; the xenograft fiber assay was performed in duplicate. Statistical analysis according to two-tailed Mann-Whitney test; *** p<0.001. Mean and 95% confidence intervals are shown.

FIG. 52E: Model of BRCAness and chemosensitivity. During stress, BRCA-deficient cells fail to effectively restrain replication, leading to ssDNA gaps that determine chemosensitvity: BRCAness. These cells acquire chemoresistance by eliminating the ssDNA gaps, either by gap filling, or by restoring fork slowing.

FIG. 53 presents exemplary data showing that depletion of SMARCAL1 or inhibition of MRE11 protects replication forks, but does not suppress ssDNA gaps or predict poor patient response.

FIG. 53A: Western blot confirms SMARCAL1 is depleted by shRNA as compared to NSC.

FIGS. 53B and 53C: Schematic and quantification of CldU track length shows that PEO1 with depleted SMARCAL1 or inhibited MRE11 protects forks after exposure to replication stress. Each dot represents one fiber. Experiments were performed in biological duplicate with at least 100 fibers per replicate. Statistical analysis according to two-tailed Mann-Whitney test; *** p<0.001. Mean and 95% confidence intervals are shown. D) cell survival assay confirms that SMARCAL1 depletion or MRE11i in PEO1 cells does not confer cisplatin resistance.

FIG. 54 presents exemplary data of:

FIG. 54A: Western blot analysis with the indicated antibodies of lysates from Control, BRCA1 K/O and BRCA1/53BP1 K/O RPE1 cells.

FIG. 54B: Cell survival assays for Control, BRCA1 K/O and BRCA1/53BP1 K/O RPE1 cells under increasing concentrations of olaparib. Data represent the mean percent±SD of survival for each dot.

FIG. 54C: Schematic and quantification of mean ssDNA intensity for indicated cell lines following CldU pre-labeling and olaparib release (10 μM, 2 hours).

FIG. 55 presents exemplary data that TLS as a gap suppression mechanism in cancer that can be re-sensitized by using the TLSi.

FIGS. 55A and 55B: Quantification of the colony formation assay after dose dependent treatment with ATR and WEE1i alone or in combination with TLSi.

FIG. 55C: Model summarizing that TLS is a replication stress avoidance mechanism in cancer. Experiments were performed in biological triplicate. Bars represent the mean±SD. Statistical analysis according to two-tailed Mann-Whitney test. All p values are described in Statistical methods.

FIGS. 56A-D presnet exemaplary data showing that TLSi as a promising cancer therapy. Representative images of the colony formation assay after dose dependent treatment with ATR and WEE1i alone or in combination with TLSi.

FIG. 57 presents exemplary data showing a therapeutic range (0.5 uM Olaparib) reveals that replication gaps predict PARPi sensitivity:

FIG. 57A: Schematic of assay design and antibody detection of ssDNA.

FIG. 57B: Quantitation of ssDNA and EdU incorporation.

While control and FANCJ K/O cells are not sensitive to 0.5 uM Olaparib, BRCA1 K/O RPE cells are sensitive (See FIG. 26) and this sensitivity correlates with ssDNA increasing in EdU positive-replicating cells as detected in non-denaturing immunofluorescence following CldU labeling and EdU pulse.

FIG. 58 presents exemplary data showing RPA exhaustion (replication catastrophe) predicts PARPi-sensitive cells.

FIG. 58A: RPE1 cells are treated as indicated and stained for gH2AX/Chromatin bound-RPA. Dashed lines indicate maximum gH2AX levels in untreated Control cells. Cells higher than those are marked as red for replication catastrophe and calculated for percentages. Each dot represents one cell; cells (n=1500±300) are collected from two independent experiments.

FIG. 58B: A model that depicts results indicating that BRCA1 K/O cells having wide-spread replication gaps that exceed the capacity of ssDNA binding protein, RPA to bind and protect and therefore DNA undergos degradation by nucleases causing a greater induction of g-H2AX following PARPi treatment

FIG. 59 presents exemplary data hsowing that dependence on RPA predicts sensitivity to PARPi.

FIG. 59A: Immunoblot shows RPA depletion in the distinct RPE cell lines.

FIG. 59B: Relative PARPi sensitivity in cell survival assays.

FIG. 60 presents exemplary data showing that elevated PAR predicts PARPi sensitivity BRCA1 K/O cells.

FIG. 60A: Representative images of immunofluorescence for RPE1 cells stained for poly (ADP-ribose) (PAR) and EdU after incubation with PARG inhibitor (10 μM, 20 min).

FIG. 60B: Quantification of mean PAR intensity and EdU are plotted as indicated. Dashed lines indicate maximum PAR level in Control cells. Cells higher than those are calculated for percentages. Each dot represents one cell; at least 500 cells are collected from three independent experiments.

FIG. 61 presents exemplary data showing a representative techniques to analyze ssDNA lesions with dynamic molecular combing.

FIG. 61A: Labeled genomic DNA from human cancer cells combed uniformly onto a silanized coverslip with dynamic molecular combing for analysis by fluorescence microscopy.

FIG. 61B: Atomic force microscopy of combed genomic DNA from human cancer cells to identify dsDNA and ssDNA regions on a silanized coverslip.

### DETAILED DESCRIPTION OF THE INVENTION

The present invention is related to the field of cancer therapy. In particular, the invention predicts the efficacy of a particular cancer therapy. The number of single stranded deoxyribonucleotide gaps near a replication fork induced by chemotherapeutic replication stress is correlated with the clinical efficacy of a chemotherapeutic agent. Consequently, a patient biopsy can be used to predict the in vivo chemotherapeutic efficacy before a cancer therapy is clinically administered.

In one embodiment, the present invention contemplates a method for directly scoring single stranded DNA gaps in cancer cell nucleic acids. In one embodiment, the number of ssDNA gaps induced by a chemotherapeutic agent is correlated with clinical efficacy. In one embodiment, an in vitro method accurately predicts the chemotherapeutic efficacy of a cancer therapy before the therapy is administered to the patient. Although it is not necessary to understand the mechanism of an invention it is believed that these ssDNA gaps are a core lesion that confers sensitivity to chemotherapy. In contrast, conventional biomarkers to DNA repair proteins rely on an incorrect model of chemotherapy (e.g., double strand DNA breaks), and therefore provide an indirect and inaccurate assessment of whether cells will be resistant to chemotherapy.

In one embodiment, the present invention contemplates an in vitro test to predict tumor response to therapy (e.g., breast tumors or ovarian tumors). In one embodiment, the test suggests whether other cancer therapies are also predicted to be effective. Although it is not necessary to understand the mechanism of an invention, it is believed that after performing the presently disclosed method, a medical practitioner is informed as to whether a particular tumor will, or will not, respond to a given therapy. This knowledge permits a real-time preemptive modification and/or improvement of a patient's drug regimen. Currently, physicians and patients have to wait weeks or months to determine if a drug is working, and they only know that a treatment is failing when the tumor grows larger, wasting precious time and allowing the disease to progress to a more dangerous state. The presently disclosed method allows physicians and patients to preemptively move to a different treatment to which the tumor is known sensitive. Such a method improves clinical outcomes and prevents unnecessary disease progression resulting from the conventional trial and error process. Another advantage of the presently disclosed method is that patient's are not subject to enduring highly toxic rounds of needless chemotherapy, only to realize after months of suffering that the chemotherapy was never going to be effective against their tumor in the first place.

In one embodiment, the present invention contemplates methods to find therapies that restore clinical response to chemotherapies. For example, when tumors develop cisplatin resistance, the method can screen for drugs that restore cisplatin sensitivity so that patients can be treated effectively. As disclosed herein, cancer cells that suppress ssDNA gap induction are resistant to chemotherapy, thus the present methods can screen for drugs that restore ssDNA gaps. In one embodiment, these restorative drugs block ssDNA gap suppression pathways such as those mediated by translesion synthesis (TLS) or replication fork restraint regulated by BRCA1/2 and/or RAD51 proteins.

The present invention has commercial applicability in the development of biomarkers, monotherapies, and combination therapies to determine chemotherapeutic efficacy before going to market. In addition, the medical community at large enjoys an improved patient outcome. Patients are prevented from having to undergo toxic and painful therapy only to find out it failed months later and was a mistake to endure which provides an overall social and community benefit and lowering the overall medical burden. For example, insurance expenditures are reduced because the presently disclosed method prevents unnecessary expensive and highly toxic chemotherapy.

### I. Nucleic Acid Replication Stress and Cancer

DNA replication stress responses are believed activated by a range of conditions including oncogene expression'. Replication stress triggers the slowing and remodeling of DNA replication forks into reversed fork structures. Local fork dynamics mediate a global replication arrest signal that halts replication even in regions not directly confronting DNA stress or lesions'. The replication stress response causes cells to either undergo replicative senescence or engage in DNA repair or other transactions that restart stalled DNA replication forks to recover global replication

By limiting replication to optimal conditions, the replication stress response serves as a barrier to cancer⁴. Thus, tumorigenesis requires replication stress response subversion by mechanisms that are not fully understood. Further complicating an understanding of how replication stress is overcome, disruption of tumor suppressors or checkpoint responses in cancer can also enhance replication stress. In particular, tumorigenesis is associated with inactivation of the classical tumor suppressor p53. Both cell cycle regulation and apoptosis are disrupted⁵. However, p53 loss also leads to enhanced replication stress because it promotes replication restart⁶. Therefore, loss of p53 may initially enhance replication stress. Cancer is also thought to gain a foothold by loss or inactivation of checkpoint signaling pathways. Indeed, inhibition of the S-phase checkpoint kinase ATR (taxia telangiectasia and Rad3-related) allows cells to replicate during stress, but this also causes wide-spread ssDNA induction that reduces cell fitness^{3,7}. Thus, ATR inhibitors are a promising cancer therapy^{8,9}. These findings support the conclusion that a failure to slow or pause replication during stress is not a productive means for tumorigenesis because ssDNA gaps are toxic.

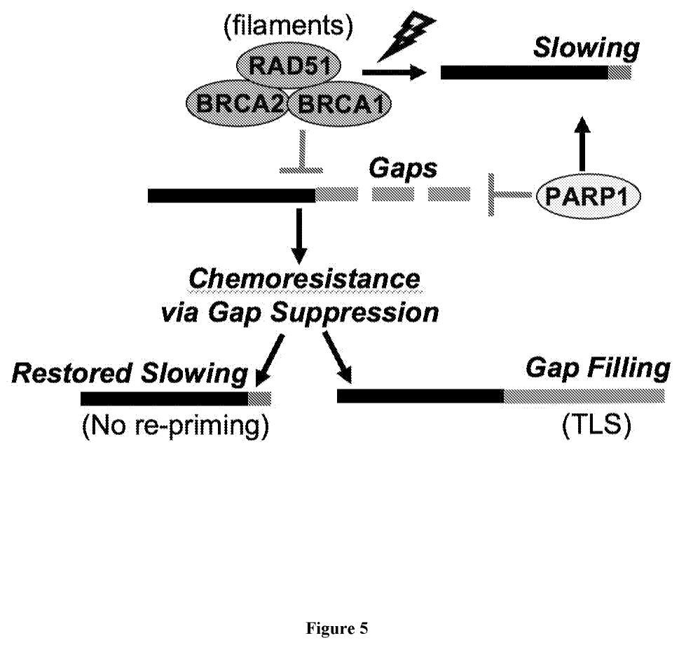

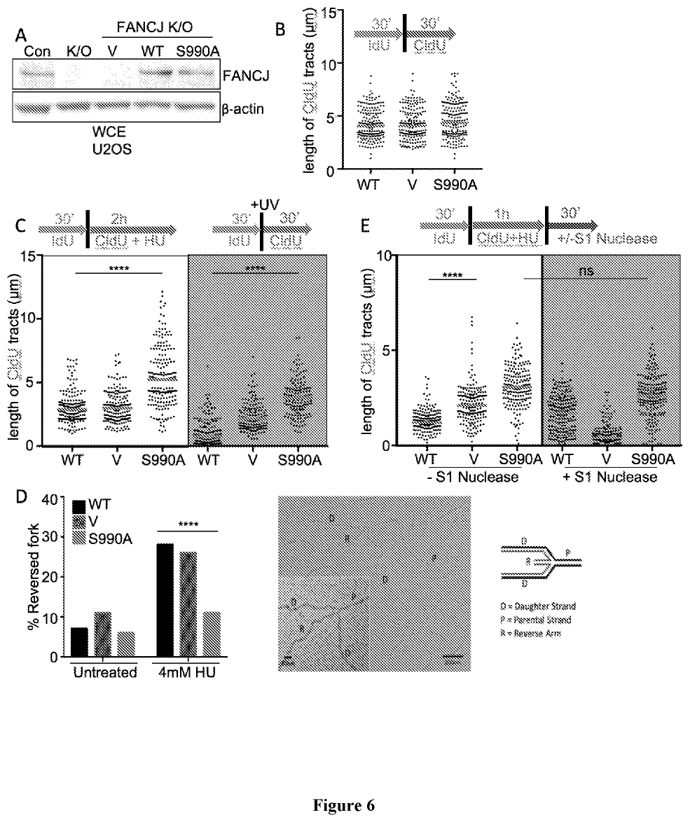

To test a hypothesis that TLS avoids the replication stress response, TLS was induced followed by an analysis of replication fork dynamics. See, FIG. 5. It was previously found that TLS is induced by the DNA helicase FANCJ when its ability to bind BRCA1 is blocked¹³. Here, the FANCJ interaction-defective BRCA1 mutant (FANCJ^{S990A}) was employed to promote TLS via polymerase polh¹⁴. FANCJ K/O U2OS osteosarcoma cancer cells were complemented and, as before, elevated cisplatin resistance compared to vector control¹⁴. See, FIG. 6A. The replication tract lengths were then measured using DNA fiber spreading analysis. This protocol involves labeling live cells with DNA analogues followed by collection of genomic DNA that is spread on glass slides for analysis of replication tracts. In particular, cells were labeled with sequential pulses of iodo-2-deoxyuridine (IdU) and 5-chloro-2-deoxyuridine (CldU) and the DNA tract lengths were measured. DNA fiber spreading analysis revealed that the complemented cell lines in unchallenged conditions had similar tract lengths. See, FIG. 6B. When CldU labeling was co-incident with hydroxyurea (HU) treatment or just following ultraviolet radiation (UV), cells with FANCJ^{WT} or vector had an expected reduction in tract lengths as compared to untreated. While overall tract length in cells with FANCJ^{S990A} also shortened with replication stress, tracts were longer than in the other U2OS cell lines suggesting that replication fork slowing was perturbed by induction of TLS in both cancer and non-cancer cells. See, FIG. 6C.

To investigate the effect of TLS on the fine replication fork architecture, psoralen cross-linking coupled to reversed fork intermediates were detected with electron microscopy (EM) with or without elevated TLS. Following 4 mM HU treatment, a substantial fraction of reversed replication forks were found in FANCJ^{WT} and empty vector U2OS cells (˜26% and 23%, respectively). On the other hand, FANCJ^{S990A} U2OS cells had a significantly lower frequency of fork reversal events, ˜11%. See, FIG. 6D. Collectively, these results strongly suggest that TLS restricts restriction fork reversal.

To test the hypothesis that TLS maintains continuous replication without ssDNA gaps, replication tracts were analyzed with or without S1 nuclease treatment. See, FIG. 6E. The S1 nuclease identifies and degrades DNA fibers with ssDNA gaps not observable in the standard DNA fiber assay₁₅. As before, during stress FANCJ^{S990A} complemented cells generated significantly longer tracts than FANCJ^{WT} U2OS cells suggesting a failure to properly slow replication during stress. With HU treatment restricted to 1 h, vector cells had longer tracts as compared to FANCJ^{WT} U2OS cells. This initial failure to slow replication rate appeared to induce ssDNA gaps. Specifically, the vector tract length reduced significantly with the addition of S1 nuclease whereas FANCJ^{WT} cells did not have a significant change in the CldU tract length. In contrast, the FANCJ^{S990A} U2OS cells had similar tract lengths in the absence or presence of the S1 nuclease indicating that TLS disrupts slowing without ssDNA gap induction. See, FIG. 6E. Collectively, these findings indicate that despite exogenous stress, replication forks are able to continue replication without gap induction when TLS is activated.

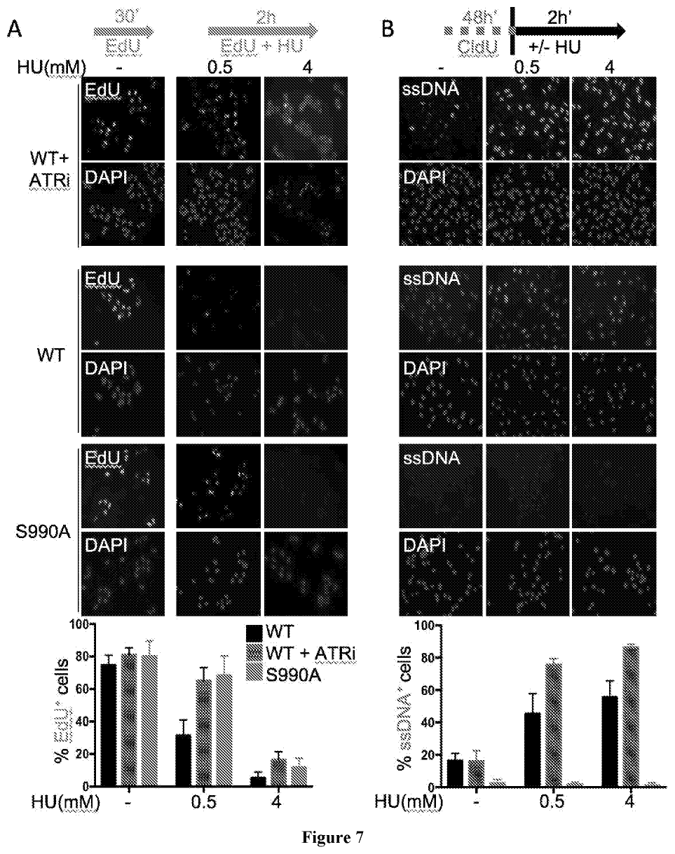

Local fork slowing and remodeling is believed involved in the global arrest of DNA replication³. Thus, TLS interference with replication fork dynamics in response to stress may also disrupt the global arrest of DNA replication. To test this hypothesis, an asynchronous population of complemented U2OS cell lines were either left untreated or treated with varying doses of HU while also being labeled with 5-ethynyl-2-deoxyuridine (EdU) to mark active replication. The efficiency of replication was quantified by scoring the number of EdU positive cells. EdU incorporation was similar in unperturbed conditions; however, HU treatment arrested replication in the FANCJ^{WT}, but not in the FANCJ^{S990A} U2OS cells. See, FIG. 7A. Despite continued replication, ssDNA gaps were not detected when analyzed under non-denaturing conditions following 48 h of CldU incorporation. See, FIG. 7B. As expected, treatment of cells with an ATR inhibitor (e.g.,VE-821; Selleckchem) promoted replication during stress, but with significant ssDNA gap induction^{3,7}. Cf., FIGS. 7A and 7B.

To validate TLS-dependent replication and ssDNA gap suppression, TLS was inhibited to determine if any observed changes in the replication stress response were curtailed. A small molecule inhibitor that targets the REV1 C-terminus that binds TLS polymerases was employed^{16,17}. Replication forks were monitored during HU treatment and in the presence or absence of a TLS inhibitor. See, FIG. 6. As previously seen, the replication tract lengths under HU treatment in U2OS cells with FANCJ^{S990A} were significantly longer as compared to an empty vector or FANCJ^{WT}. However, with the addition of TLSi 1 (20 μM) these tract lengths in FANCJ^{S990A} U2OS cells dramatically shortened, while tract lengths in empty vector or FANCJ^{WT} cells did not change. See, FIG. 8A. Moreover, analysis of global replication demonstrated that the unrestrained replication despite HU treatment observed in the FANCJ^{S990A} cells was disrupted by TLSi 1. See, FIG. 8B. By simultaneously scoring both EdU incorporation along with ssDNA induction, unrestrained replication in HU treatment was reduced by TLSi 1 and ssDNA gaps were elevated. See, FIG. 8B and FIG. 8C.

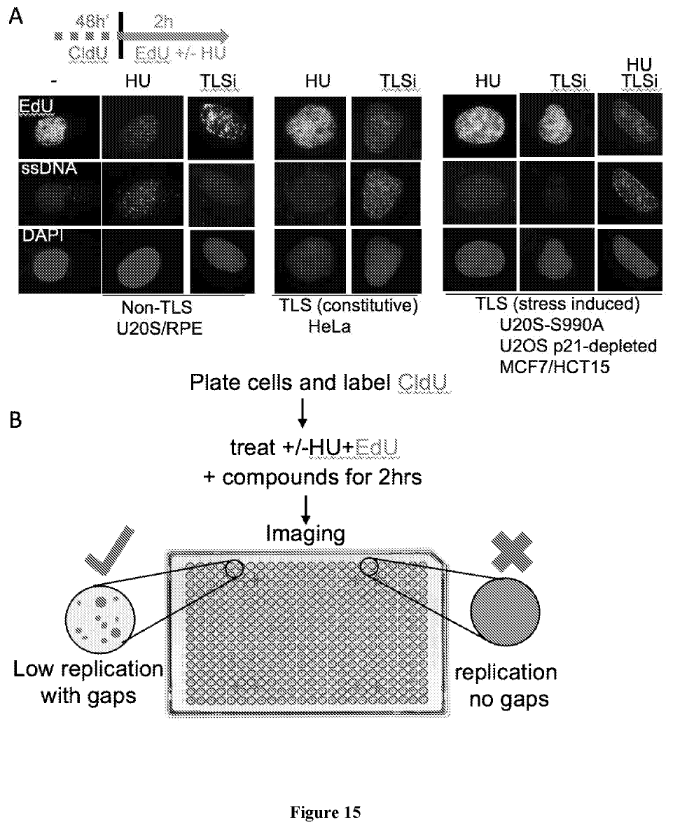

Notably, analysis of global replication demonstrated that TLSi 1 did not interfere with replication in either of the U2OS cell lines in unchallenged conditions. See, FIG. 8B. Furthermore, TLSi 1 did not alter the cisplatin sensitivity of empty vector or FANCJ^{WT}, only the FANCJ^{S990A} U2OS cells were sensitized. See, FIG. 8C. These findings further validate that the FANCJ^{S990A} mutant promotes cisplatin resistance via a REV1-dependent TLS mechanism and indicate that a combined assay is a useful tool to assess whether cancer cells rely on TLS to replicate in a continuous manner and define compounds that block TLS¹⁴ See, FIG. 15.

### II. Translesional Synthesis (TLS)

One pathway for tolerating replication stress, at least in the form of DNA lesions, is translesion synthesis (TLS). TLS is facilitated by mono-ubiquitylation of proliferating cell nuclear antigen (PCNA). TLS polymerases are recruited to PCNA mono-ubiquitylation via their ubiquitin binding domains and lesions that hinder replication can be bypassed when the replicative polymerases are not functional or physically blocked¹⁰. TLS polymerases also have the ability to replicate through abnormal secondary DNA structures that form during replication stress¹¹. Given the findings that PCNA polyubiquitylation is required for fork slowing and reversal¹², PCNA monoubiquitylation that promotes TLS could oppose the replication stress response.

A. TLS Replisome Modifications

Although it is not necessary to understand the mechanism of an invention, it is believed that a cancer cell promotes replication during stress by rewiring a replisome to enhance TLS factors and reduce fork slowing factors. It is further believed that TLS drives ssDNA gap suppression during stress that is orchestrated by changes in the composition of the replisome. By performing single-molecule DNA fiber assays, electron microscopy, and immunoblot analysis, the presently disclosed data shows how replication fork dynamics are altered and detection of proteomic changes in cells with a TLS. For example, a TLS may be activated or inactivated in distinct ways and cell systems, which can construct a proteomic model of TLS. Some embodiments of the present invention disclose candidate TLS biomarkers identified by quantitative proteomics and Identification of Proteins On Nascent DNA (iPOND) to design TLS therapeutics to target and disrupt TLS.

To analyze the protein landscape of TLS, a TLS cell may be compared to a non-TLS cell. For example, U2OS cells comprising either FANCJ^{WT} (non-TLS) may be compared to the pro-TLS mutant FANCJ^{S990A} (TLS cell). In addition, inhibition of a TLS cell may be assessed by comparing FANCJ^{S990A} U2OS cells without or with TLSi 1. Alternatively, a U2OS cell TLS may be activated via p21 depletion. Cells can be cultured in either “light” and/or “heavy” stable isotopic versions of arginine and/or lysine, in which the ¹²C and ¹⁴N atoms are replaced by ¹³C and ¹⁵N atoms⁶⁰. The percentage of heavy amino acid incorporation are determined by MS analysis⁵².

A peptide containing a heavy amino acid results in a known mass shift compared with the same peptide that contains the light version of the amino acid. Therefore, each peptide identified has a light and a heavy peak; the ratio of these two peaks corresponds to the relative abundance of the peptide between the light- and heavy-labeled samples. Next, equal numbers of heavy and light cells were combined and processed together for iPOND. After LC-MS/MS, raw proteomics data can be analyzed with MaxQuant®⁶⁶ to identify proteins (with at least two unique peptides in the UniProt Human Reference Proteome UP000005640) and quantify their ratios between samples. Proteins found after an EdU labeling pulse and a thymidine chase are excluded from the analysis to ensure that only proteins associated with the replication fork are included.

Data from at least three independent iPOND experiments are then combined and analyzed. For a subsequent statistical analysis, first the ratios are converted to a log 2 scale for graphing. To normalize the data, the median ratio value of each experiment is obtained and subtracted from the ratio values for that experiment. Proteins that are only identified in one experiment were excluded from analysis. Finally, the data will be analyzed using the limma®⁶⁷ statistical package for R using the lmFit, eBayes, and topTable functions to obtain p-values. The p-values will be converted to −log 10(p-value) for graphing, and the data will be presented as log 2 Ratio vs −log 10(p-value).

To validate and prioritize replisome candidates from iPOND data several criteria are considered. First, only proteins with statistically significant changes (enriched or reduced) are included and proteins that precipitate with the EdU label following a thymidine chase that are no longer with the active replisome are excluded. Second, priority is given to “node proteins” in pathways related to DNA repair or DNA replication identified using Gene Ontology Enrichment Analysis, PANTHER, and STRING. Candidates mutated or over-expressed in cancer are prioritized. Focusing on high-priority proteins, SILAC-chromatin-MS and immunoblot analysis validations are performed. Collectively, this approach reveals how TLS alters the replisome in actively replicating cells.

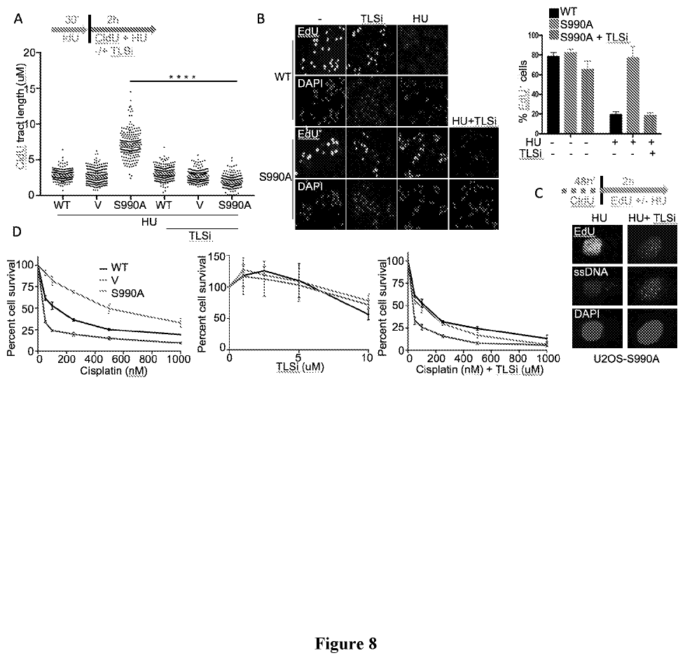

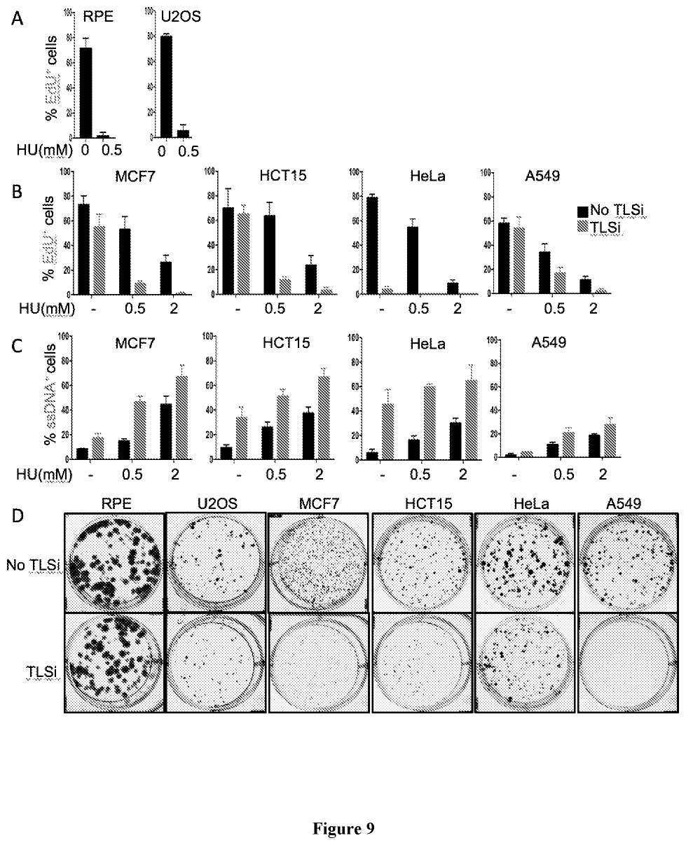

Preliminary data indicated that TLS is employed by a range of cancers to subvert the replication stress response. See, FIG. 9. These findings indicate that TLS is a vulnerability of cancer cells that can be exploited for therapy. However, mechanistic understanding for TLS regulation is lacking. To determine how to selectively inhibit TLS and not affect replication machinery in non-cancer cells, one should identify proteins associated with a TLS cancer cell replisome. Thus, the identification of components of a TLS cancer cell vs a non-TLS cancer cell might be achieved by performing a technique known as Isolation Of Proteins On Nascent DNA—Stable Isotope Labeling Of Amino Acids In Cell Culture (iPOND-SILAC)⁵⁷. This approach has previously shown that FANCJ loss significantly alters the composition of the replisome⁵².

iPOND may also be combined with mass spectrometry (MS)-based proteomics^{13,52}. The iPOND approach is highly sensitive and proteins at the level of one or two molecules per replication fork can be identified by LC-MS/MS. iPOND uses EdU to label newly synthesized DNA. Following the labeling period, treatment with formaldehyde cross-links the DNA to associated proteins. Covalent linkage of the alkyne functional group on EdU to a biotin-azide (e.g., by click reaction) provides a biotin tag for purification of DNAprotein complexes⁵⁹. Streptavidin affinity purification followed by cross-link reversal frees the DNA-associated proteins for analysis by immunoblot or MS. SILAC can also be useful because this technique provides a sensitive and quantitative approach to detect protein changes in two distinct cell populations⁶⁰.

A combination of iPOND and proteomics can provide insight into how TLS activation vs inhibition alters the replisome and associated replication fork dynamics. These data demonstrate how TLS is orchestrated in mammalian cells, provide biomarkers of TLS and identify how to disrupt TLS in cancer.

B. Chromatin Composition of TLS Activated Cells

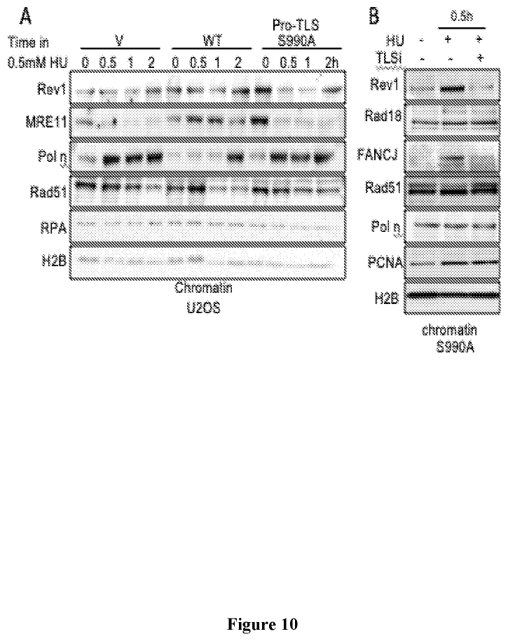

To gain an initial appreciation of how the replication stress response is subverted by TLS, an immunoblot analysis shows how TLS induction alters chromatin composition of a non-TLS cancer cell. In particular, TLS was induced by expressing FANCJ^{S990A} in non-TLS FANCJ K/O U2OS cells. These cells, as compared to empty vector or wild type-complemented cells, were analyzed for the recombinase RAD51 that also contributes to replication fork restraint and the fork degradation nuclease. Following HU treatment, both MRE11 and RAD51 expression was reduced, but TLS polymerase (polh) in the FANCJ^{S990A} cells expression was enhanced as compared to the FANCJ^{WT} U2OS cells. See, FIG. 10A. Furthermore, in the FANCJ^{S990A} cells, the TLS inhibitor displaced REV1 and FANCJ from the chromatin, but other factors analyzed were unchanged. See, FIG. 10B. These preliminary findings support a hypothesis that the TLS chromatin and replisome are modified, and in part disrupted, by the TLSi. A comprehensive evaluation of a TLS vs a non-TLS cancer replisome can provide much needed insight into how TLS is orchestrated in mammalian cells and how best to disrupt TLS in cancer.

Depletion of replication fork remodelers such as HLTF, SMARCAL1 or a slowing factor (e.g., RAD51) in BRCA-deficient cells restores replication fork protection⁶⁸. Thus, TLS may also restore replication fork protection in BRCA2 deficient ovarian cancer cells as reversed forks that are the target of MRE11 nucleases are avoided. If TLS activation protects replication forks and elevates resistance to chemotherapies such as cisplatin or PARPi, this supports findings that TLS is a mechanism of chemoresistance in BRCA-deficient cancer and highlights the involvement of targeting TLS as part of cancer therapy³³. With TLS activated, cells are expected to have a replisome chromatin composition with elevated TLS factors (e.g., polh, REV1, PCNA monoubiquitin) and/or reduced slowing/remodeling factors (e.g., FANCD2, FAN1, HLTF, RAD51)^{1,68-71}. Cells treated with low dose HU only induce a few changes in the replisome. TLS activation with higher doses of replication stress may readily activate PCNA monoubiquitination, a known biomarker of TLS⁷². However, with low dose HU treatment, the chromatin composition in TLS activated FANCJ^{S990A} expressing U2OS is distinct from controls and these changes can be disrupted by TLSi 1. In cases in which iPOND is not feasible due to low incorporation of EdU, chromatin may be compared by quantitative proteomics to identify TLS factors. See, FIG. 10.

A TLS cancer replisome may also be achieved by a phenocopy each other. Thus, replisome changes may occur following TLS activation or inhibition in distinct TLS cancer cells. This analysis could identify targets for TLS. Such data can be evaluated using immunoblot analysis on iPOND and chromatin samples or a proximity ligation assay (PLA)-based approach that measures the association of proteins to nascent DNA⁷³. Cells are labeled with EdU and subsequently treated with HU. Biotin can be conjugated to EdU by click chemistry, and PLA can used to detect protein binding to biotin-labeled nascent DNA⁷⁴. The number of biotin PLA foci can the be measured with a protein of interest with respect to the number of total replication sites marked by biotin/biotin PLA foci. To uncover a hierarchy of replisome localization, a depletion of a protein of interest may reduce the number of biotin PLA foci with another protein of interest.

C. TLS Suppression of the Replication Stress Response

It is believed that cancer cells may subvert the replication stress response by engaging the DNA damage tolerance mechanism translesion synthesis (TLS). For example, the data presented herein shows that TLS suppresses the replication stress response such that DNA replication continues despite replication stress. Moreover, small molecule inhibitors (e.g., TLS inhibitors;

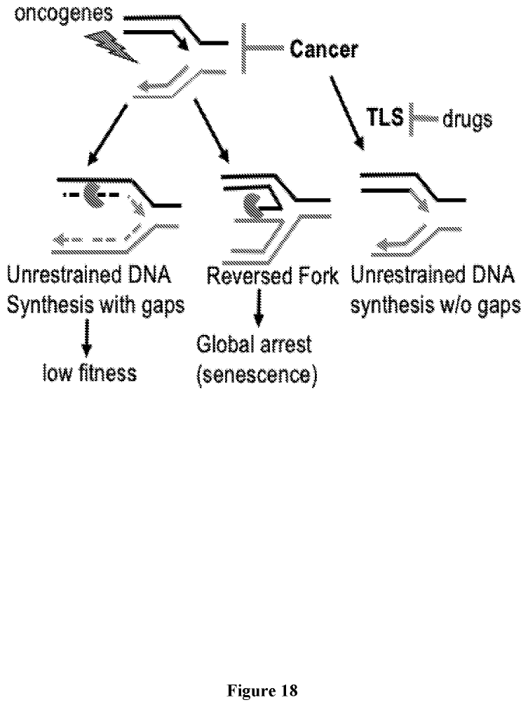

TLSi) are herein disclosed that restore the replication stress response selectively in cancer cells which are dependent on TLS. These TLSi compounds limit cancer cell replication and clonogenic capacity even without the addition of other drugs, but do not affect normal non-cancer cells. These preliminary findings suggest that TLS may be a wide-spread cancer mechanism employed to blunt inherent replication stress and maintain survival and that TLSi will selectively kill cancer cells (e.g., by apoptosis induction). See, FIG. 18. In one embodiment, the present invention contemplates that TLS is a target for cancer therapy that is highly specific and nontoxic that induces cancer cell death and prevents cancer cell development.

To verify that unrestrained replication without ssDNA gaps is a feature of TLS and not limited to TLS-driven by FANCJ^{S990A}, TLS was activated by other means in U2OS cells. For example, by depletion of p21, a known negative regulator of TLS⁶¹. TLS might also be activated by depletion of the de-ubiquitinase USP1 that removes PCNA mono-ubiquitination that promotes TLS⁶². TLS might also be activated by over-expression of the TLS factor REV1 that scaffolds TLS polymerases with its C-terminal domain to coordinate TLS⁶³.

Expression vectors comprising either a human REV1 wild-type, C-terminal mutant and/or a polymerase-inactive REV1 mutant were used to collect the following data. Following confirmation of expression or depletion in U2OS cells via immunoblot analysis, replication dynamics and global replication was analyzed. Depletion of p21 reduced p21 expression and elevated replication during stress, without ssDNA gap induction, was found using the described combined assay. See, FIG. 11. These data suggest that p21 depletion mimics expression of the FANCJ^{S990A}. To verify that TLS is activated, TLS disruption by the TLSi or depletion of TLS factors may restore the replication stress response. Indeed, unrestrained replication by p21 depletion was blocked by TLSi 1. See, FIG. 11. Here, TLS suppresses local and global replication stress responses following distinct forms of stress such as mitomycin C (MMC) or UV light, that induce lesions or aphidicolin, which similar to HU treatment, and does not induce DNA lesions.

Other cell systems that are non-TLS can be converted to TLS cells as found for U2OS cells. A non-TLS cell may be defined by an arrest of their replication and induction of ssDNA gaps (e.g., as determined in a combined assay) with a low dose (0.5 mM) HU treatment. But in the presence of a TLSi, continued replication without ssDNA induction or loss of clonogenic capacity is observed. See, FIG. 9B and FIG. 10D. In contrast, TLS-activated cells are defined by continued replication without ssDNA gap induction in the presence of a low dose HU treatment. But replication arrest, ssDNA induction, and loss of clonogenic capacity is seen when incubated with TLSi 1 either alone or with low dose HU treatment as observed in either FANCJ^{S990A} U2OS or HeLa cells. See, FIG. 9B and FIG. 10D. Non-tumorigenic, immortalized human fibroblasts and retinol pigment epithelial (RPE) cells can be tested in this assay subsequent to confirmation that these cells are non-TLS (i.e. replication arrests in low dose HU) and can be converted to TLS cells by means found to be successful in U2OS cells (i.e. p21 depletion).

D. TLS Domination in BRCA2-Deficient Cells

Reduced replication rate slowing and chromatin remodeling protects nascent DNA in replication forks from degradation. This has been reported in BRCA2 mutant cells in which reversed replication forks are not effectively protected from MRE11 nuclease degradation⁶⁴. Thus, activation of TLS in a BRCA2 mutant cell line (BRCA2-deficient PEO1 ovarian cancer cells65) by ectopically expressed FANCJ^{S990A} (p21 depletion vs controls) may prevent fork degradation. .

E. TLS and Enriched Proteins

With significant proteins/pathways upregulated/represented/validated in TLS cells, selective requirements in TLS as opposed to non-TLS cells for replication during stress and for clonogenic proficiency can be evaluated. For this approach, shRNAs targeting the proteins of interest vs controls in TLS vs non-TLS cells can confirm protein expression level changes by immunoblot. The data are compared to controls that disrupt TLS such as depletion of TLS factor, such RAD18 or TLSi 1. Replication rates are determined in a combined assay and measured for clonogenic survival. Proteins in which two or more siRNAs reproducibly reduce either parameter are advanced for further testing and validation in other TLS cells (e.g., MCF7, HeLa). With these enriched proteins, RNA expression in patient databases can determine if expression correlates with poor patient response and/or survival³³.

F. TLS Promotes Replication During Stress and Suppresses ssDNA Gaps

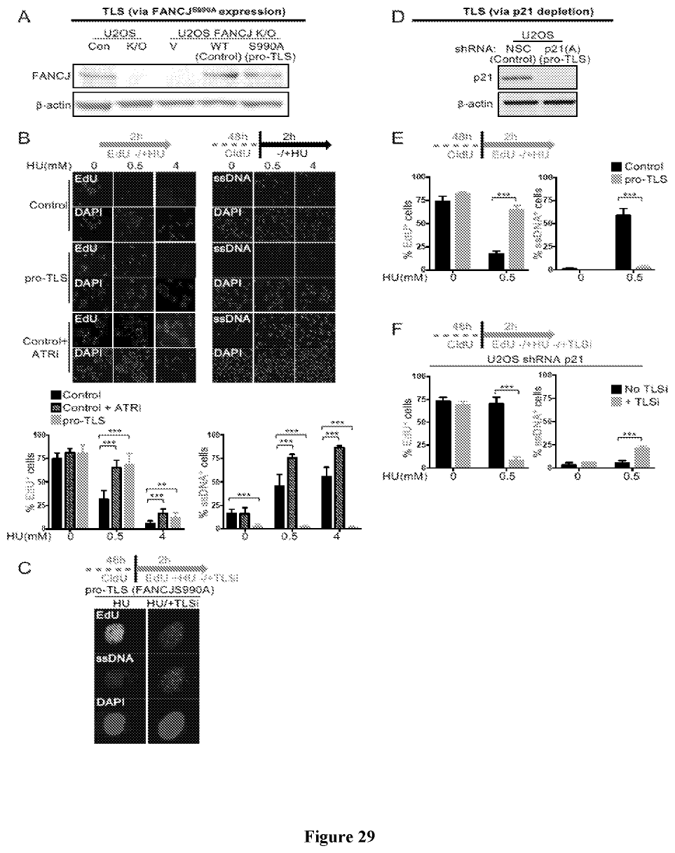

To test the hypothesis that replication during stress is possible when TLS is activated, it was sought to induce TLS and analyze replication during stress. Replication in the osteosarcoma U2OS cell line is significantly reduced upon replication stress induced by hydroxyurea (HU). Zellweger et al., “Rad51-mediated replication fork reversal is a global response to genotoxic treatments in human cells” J Cell Biol 208:563-579 (2015). It has previously been shown that the DNA helicase FANCJ promotes TLS when its BRCA1 interaction is disrupted (FANCJ^{S990A}) Thus, FANCJ CRISPR knockout (K/O) U2OS cells were complemented with FANCJ^{WT} (control), empty vector or the pro-TLS, FANCJS990A mutant. See, FIG. 29A. As expected, FANCJ^{WT} and FANCJ^{S990A} expressing U2OS cells elevated resistance to DNA interstrand crosslinking (ICL) agent, mitomycin-C (MMC), as compared to the empty vector control. See, FIG. 30;. Xie et al., “Targeting the FANCJ-BRCA1 interaction promotes a switch from recombination to poleta-dependent bypass. Oncogene 29:2499-2508 (2010).

In order to track active replication, cells were labeled with 5-ethynyl-2-deoxyuridine (EdU) and either left untreated or treated with low dose HU (0.5 mM), that activates replication stress without fully depleting nucleotide pools. Koc et al., “Hydroxyurea arrests DNA replication by a mechanism that preserves basal dNTP pools” J Biol Chem 279:223-230 (2004). Under unperturbed conditions, the EdU incorporation was similar between the (control) and (pro-TLS) U2OS cells, suggesting that TLS does not impact replication in unchallenged conditions. See FIG. 29B. Upon low dose HU treatment, it was observed that DNA replication slowed in the control complemented U2OS cells, similar to a parental U2OS cell line. However, the pro-TLS U2OS cells, continued to replicate not only in HU, but also following treatment with ultraviolet radiation (UV) suggesting that TLS can promote replication during stress. Cf, FIG. 29B and FIG. 30B.

To support the suggestion that TLS promotes replication during stress, it was sought to induce TLS by another means. The cyclin-dependent kinase inhibitor, p21, is a negative regulator of TLS Avkin et al., “p53 and p21 regulate error-prone DNA repair to yield a lower mutation load” Molecular Cell 22:407-413 (2006). See, FIG. 29D and FIG. 31A. Three distinct shRNAs demonstrated reduced expression of p21 as compared to a non-silencing control (NSC), all of which did not significantly alter replication in unchallenged conditions. See, FIG. 29E and FIGS. 31B & 31C. However, as compared to NSC, replication continued in the p21 depleted U2OS cells despite treatment with HU.

Collectively, these findings suggest that TLS enables replication during stress. It was also confirmed that ATR inhibition leads to replication during stress Mutreja et al., “ATR-Mediated Global Fork Slowing and Reversal Assist Fork Traverse and Prevent Chromosomal Breakage at DNA Interstrand Cross-Links” Cell Rep 24:2629-2642 (2018). See, FIG. 29B. Given that ATR inhibition is toxic to cells, it was considered that a difference between TLS activation and ATR inhibition was a mode of replication during stress. Specifically, ATR inhibition is known to cause ssDNA induction and TLS is associated with ssDNA suppression Couch et al., “ATR phosphorylates SMARCAL1 to prevent replication fork collapse” Genes Dev 27:1610-1623 (2013); and Yang et al., “Diverse roles of RAD18 and Y-family DNA polymerases in tumorigenesis” Cell Cycle 17:833-843 (2018).

To test whether replication during stress differed by ssDNA gap induction, non-denaturing immunofluorescence was performed following incorporation of 5-chloro-2′-deoxyuridine (CldU). In the presence of HU, it was found that ssDNA was significantly induced in the control, but not in the pro-TLS cells (e.g., either S990A mutant or p21-depleted). See, FIGS. 29B and 29E. Moreover, wide-spread ssDNA developed in the control cells treated with ATRi.

Collectively, these findings indicate that TLS, unlike ATR inhibition, promotes replication during stress without ssDNA induction. Further validating that TLS was activated and required for replication during stress, replication was arrested and ssDNA gaps developed when pro-TLS cells were treated with a TLS inhibitor (TLSi) that targets the C-terminus domain of REV, that scaffolds several TLS polymerases. Korzhnev et al., “Targeting the Translesion Synthesis Pathway for the Development of Anti-Cancer Chemotherapeutics” J Med Chem 59:9321-9336 (2016); and Sail et al., “Identification of Small Molecule Translesion Synthesis Inhibitors That Target the Revl-CT/RIR Protein-Protein Interaction” ACS Chem Biol 12:1903-1912 (2017). Notably, the TLSi did not interfere with the replication in unchallenged cells suggesting that TLS-dependent replication was dependent on the stress induced by HU treatment. See, FIGS. 29C & 29E, FIGS. 30C & 30D, and FIG. 31D. Moreover, the TLSi specifically sensitized the pro-TLS cells to cisplatin indicating the selectivity of TLSi for TLS dependent cells. See, FIG. 30E.

G. TLS Limits Replication Fork Slowing, Reversal, Degradation and Gap Induction

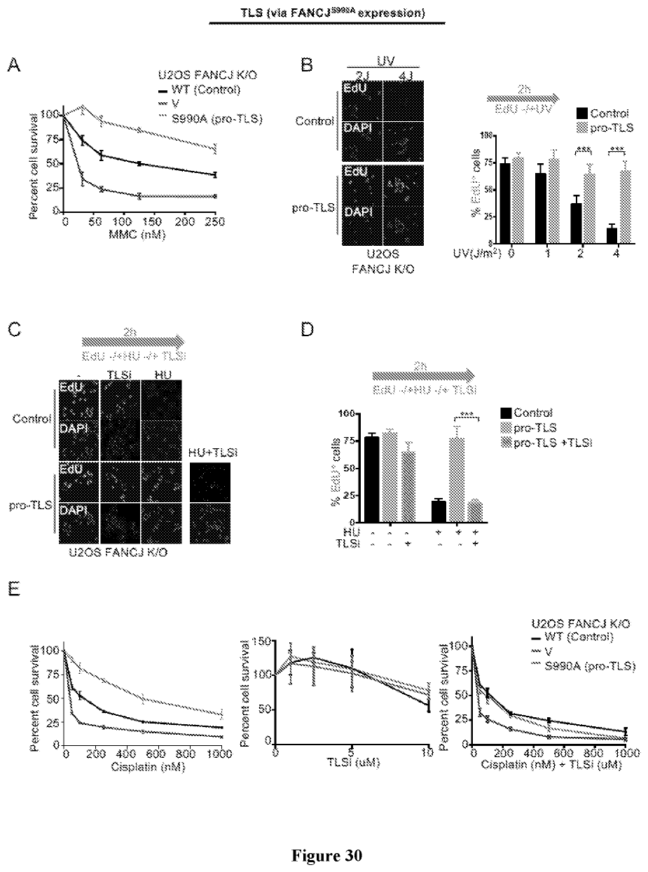

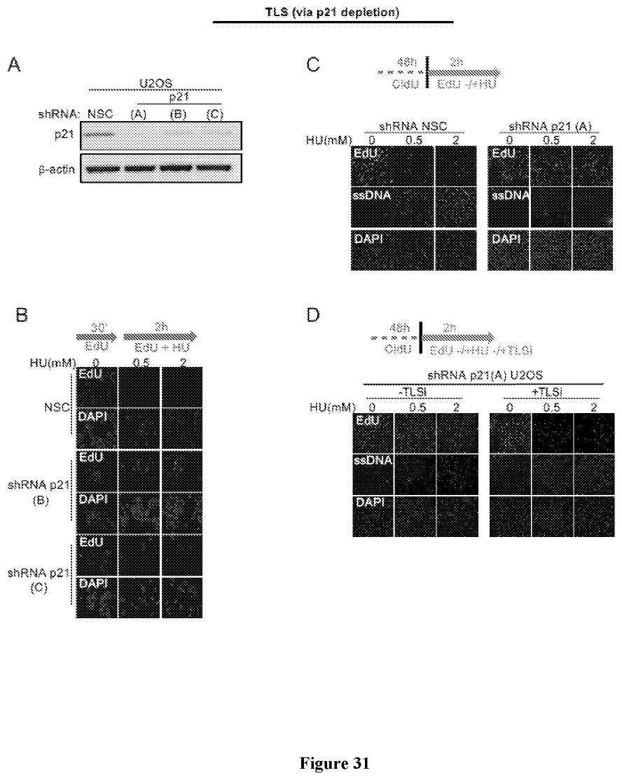

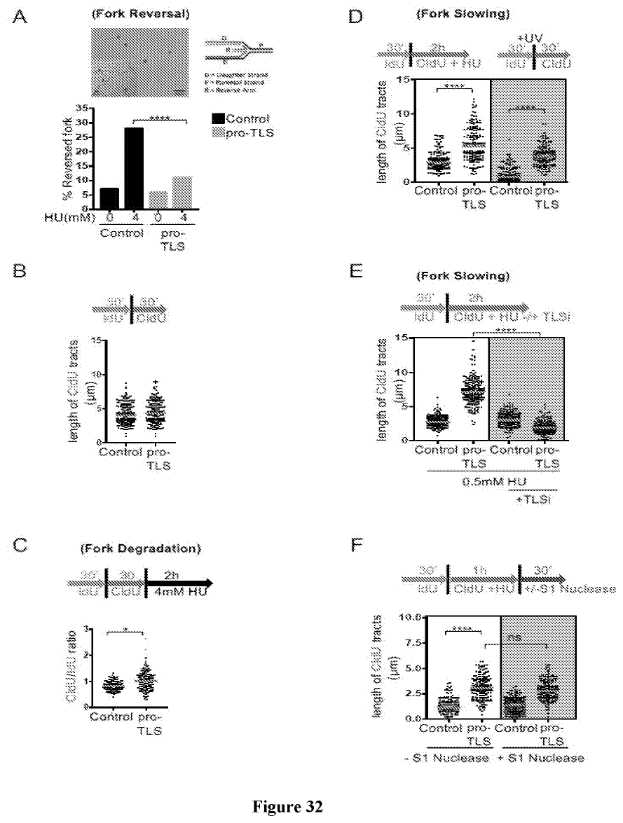

To understand a mechanism of how TLS maintains global replication during stress, it was considered that ATR inhibition might achieve this via a block to fork remodeling. To test whether TLS impacts fork dynamics, it was sought to determine if TLS impacted the fine replication fork architecture. Psoralen cross-linking was employed coupled with replication fork dynamics by performing Electron Microscopy (EM) to look at the frequency of reversed fork intermediates in cells with or without TLS induction. Following 4 mM HU treatment, a significant increase in the reversed fork structures was found in the control, whereas pro-TLS cells exhibited significantly lower frequency of fork reversal events. See, FIG. 32A. Collectively, these results suggest that TLS restricts fork reversal.

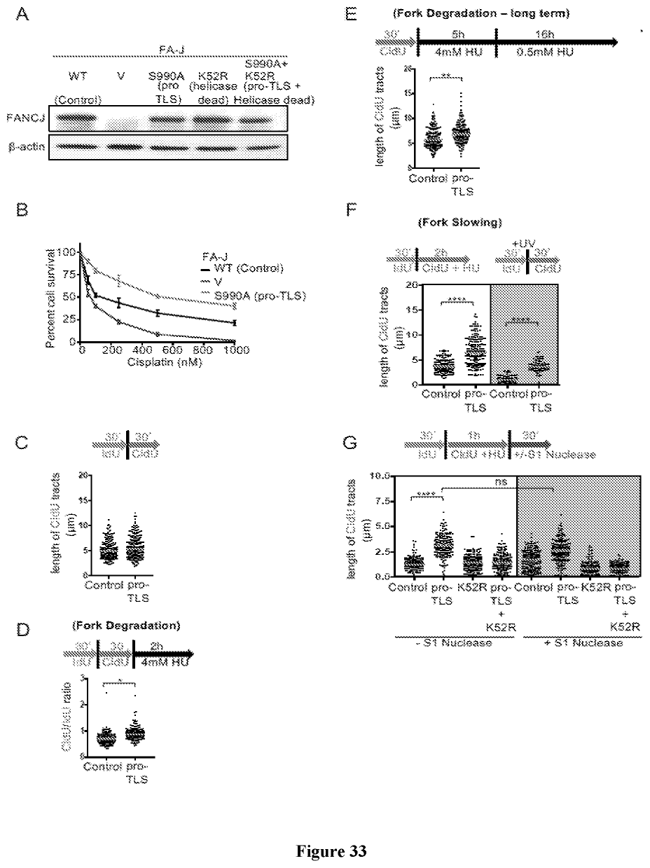

Next, it was assessed if TLS activation altered other aspects of replication fork dynamics by performing DNA fiber spreading analysis. Replication tract lengths were measured in control and pro-TLS U2OS or FANCJ-null FA-J patient immortalized fibroblasts cells complemented with FANCJ^{WT} (control), empty vector or the pro-TLS FANCJ^{S990A} mutant. The data show that FA-J cells with either FANCJ^{S990A} or FANCJ^{WT} as compared to an empty vector control gained cisplatin resistance. See, FIGS. 33A & 33B. In unchallenged conditions, the control or pro-TLS cells labeled with iodo-2-deoxyuridine (IdU) followed by CldU displayed similar tract lengths, indicating again that TLS did not impact normal replication progression. See, FIG. 32B and FIG. 33C.

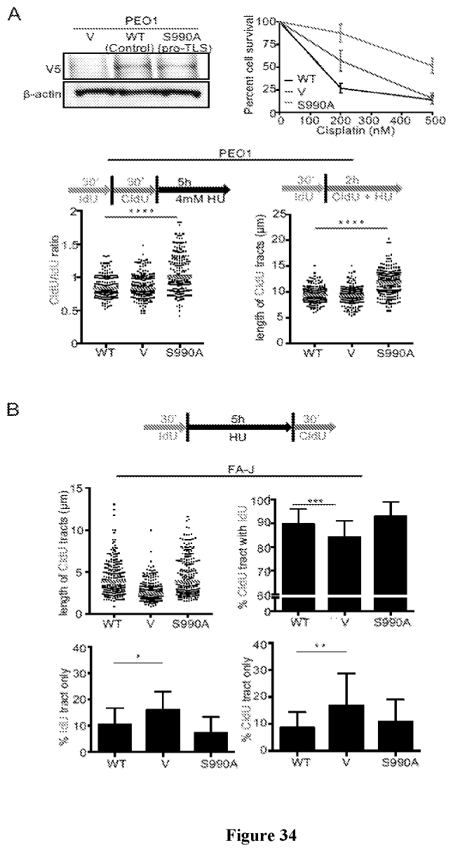

To measure replication fork degradation, the ratio of CldU to IdU tract lengths were analyzed following sequential pulses with IdU and CldU followed by HU treatment. Schlacher et al., “Double-strand break repair-independent role for BRCA2 in blocking stalled replication fork degradation by MRE11” Cell 145:529-542 (2011). See, FIG. 32C. Notably, as compared to control, the pro-TLS cells had a modestly enhanced CldU to IdU tract length ratio, consistent with less fork degradation. See, FIG. 32C and FIG. 33D. Pro-TLS cells also maintained fork integrity following prolonged replication stress. See, FIG. 33E. Moreover, ectopic expression of the pro-TLS mutant in BRCA2-deficient PEO1 ovarian cancer cells enhanced not only fork protection, but also cisplatin resistance. Sakai et al., “Functional restoration of BRCA2 protein by secondary BRCA2 mutations in BRCA2-mutated ovarian carcinoma” Cancer Res 69:6381-6386 (2009). See, FIG. 34A. Collectively, these findings indicate that TLS provides fork protection through suppression of fork remodeling. Prior to fork reversal, replication slows in response to stress. Indeed, the replication slowing (concomitantly shortening the replication tracts) in control cells is readily observed when HU or UV treatment is co-incident with analogue incorporation. See, FIG. 30D and FIG. 33F. As compared to control cells, it was observed that pro-TLS cells did not shorten DNA tracts as robustly, unless TLS was inhibited. See, FIG. 30E.

Conceivably, a more rapid restart of stalled replication forks and/or the firing of new replication origins could generate longer tracts in pro-TLS cells. However, this did not appear to be the case, based on an analysis of replication restart and new origin firing. While FANCJ null cells displayed replication restart defects and the aberrant activation of dormant origins, control and pro-TLS cells readily incorporated CldU following a brief HU arrest into a prior labelled IdU tract, indicative of replication restart from the pre-existing fork. See, FIG. 34B. Together, these findings suggest that TLS interferes with replication fork slowing, but does not alter replication restart or dormant origin firing in response to stress.

To test the hypothesis that TLS maintains replication during stress without inducing ssDNA gaps, replication tracts were analyzed as before, but in presence or absence of S1 nuclease treatment. The S1 nuclease identifies and degrades DNA fibers with ssDNA gaps otherwise not observable in the standard DNA fiber assay. Quinet et al., “DNA Fiber Analysis: Mind the Gap!” Methods Enzymol 591:55-82 (2017). .As before, during stress, pro-TLS cells generated significantly longer tracts than control cells and these tracts were maintained even after S1 nuclease treatment, indicating that failure to slow is not associated with ssDNA gaps. See, FIG. 31F and FIG. 33G. In contrast, tract shortening with S1 nuclease occurred in FANCJ null cells or in cells expressing the FANCJ^{K52R} (helicase dead) or TLS inactivating mutant FANCJ^{S990A+K52R} (pro-TLS+helicase dead). See, FIG. 33G. Cantor et al., “BACH1, a novel helicase-like protein, interacts directly with BRCA1 and contributes to its DNA repair function” Cell 105:149-160 (2001). Collectively these findings indicate that in response to stress, TLS suppresses fork reversal, degradation, and slowing without replication gap induction.

H. TLS Subverts the Replication Stress Response to Promote Cancer Fitness

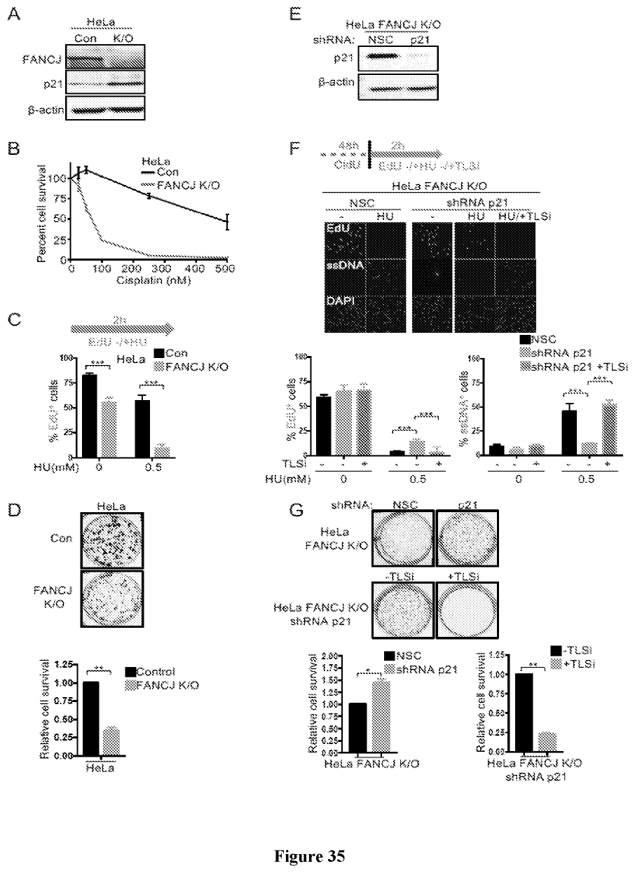

To test the idea that TLS plays a role in cancer, it was first considered that FANCJ could be co-opted in cancer to aberrantly promote TLS and reduce replication stress. Indeed, despite being a tumor suppressor, FANCJ is also overexpressed in cancer cells, such as the breast cancer cell line (MCF7), in which it was found FANCJ K/O was not achievable, suggesting that FANCJ could be involved in MCF7 cell viability. Eelen et al., “Expression of the BRCA1-interacting protein Brip1/BACH1/FANCJ is driven by E2F and correlates with human breast cancer malignancy” Oncogene 27:4233-4241 (2008). Moreover, K/O of FANCJ in the endometrial cancer cell line (HeLa) significantly reduced DNA replication and impaired clonogenic capacity. See, FIGS. 35A-D. Interestingly, along with cisplatin sensitivity, p21 levels were also elevated in the FANCJ K/O HeLa cells. Furthermore, p21 depletion improved replication, fitness and suppressed ssDNA gaps, unless TLS (via p21 depletion) was also inhibited by the TLSi. See, FIGS. 35E-G. These findings indicate that FANCJ suppresses p21 and that this in turn activates TLS in HeLa cells.

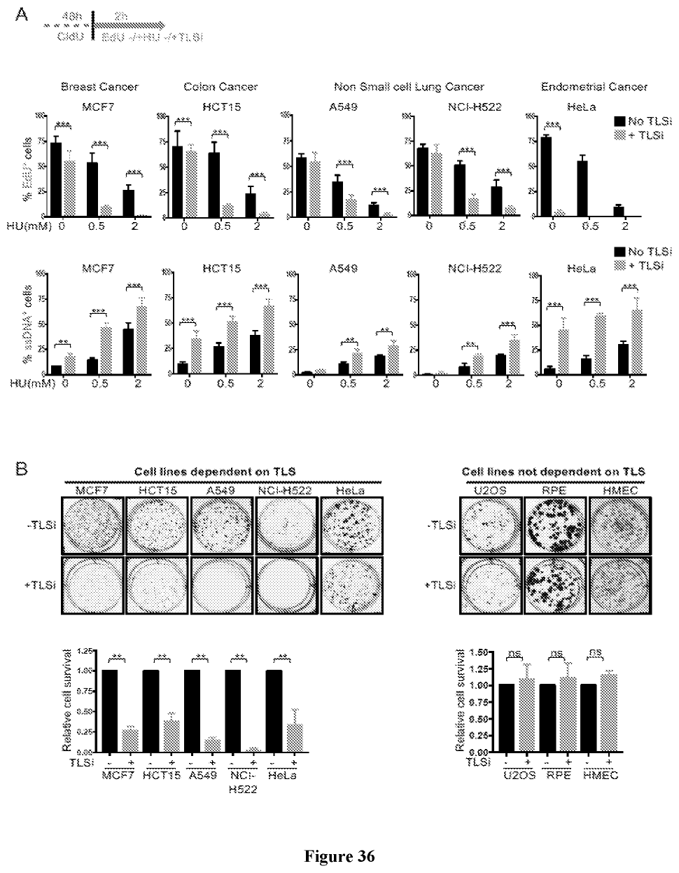

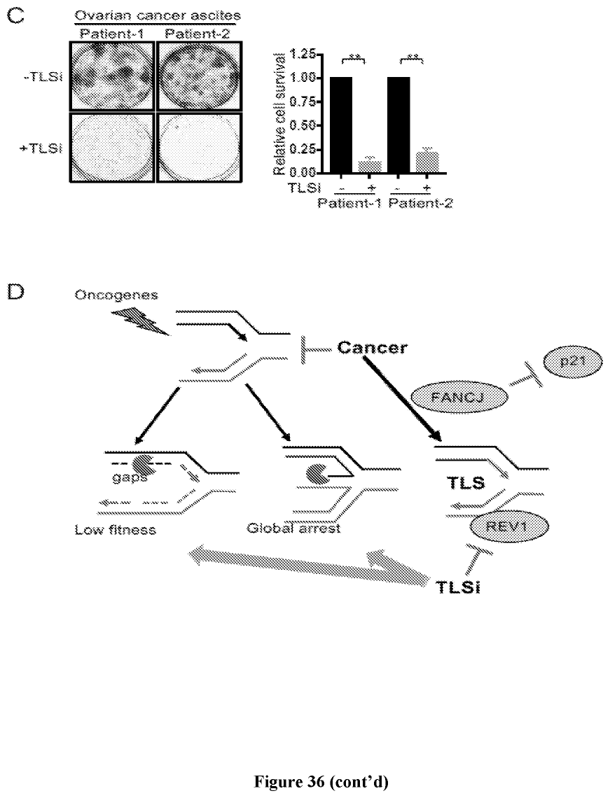

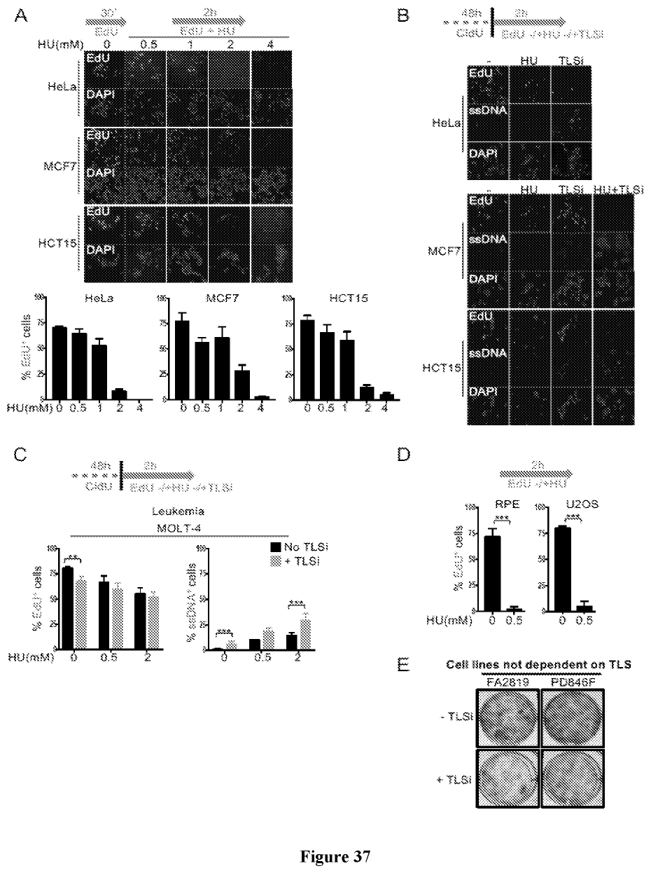

Rather than deleting FANCJ in numerous cancer cell lines, it was sought to screen cancer cells using a TLSi to ascertain whether activation of TLS is a widespread phenomenon in cancer cells. A panel of different cancer cell types was analyzed for their ability to replicate during stress. Remarkably, it was found that replication robustly continued not only in HeLa cells, but also in MCF7, the colon cancer cell line (HCT15), the lung cancer cell lines (A549 and NCI-H522), and the leukemia cell line (MOLT-4). See, FIG. 36A and FIG. 37A-C. However, similar to U2OS cells, the retinal pigment epithelial (RPE) immortalized cell line ceased to replicate in the presence of 0.5 mM HU. See, FIG. 37D.

The data presented herein shows that replication during stress exhibited by cancer cell lines was curtailed by a TLSi. Indeed, when a TLSi was co-incubated with HU, the unrestrained replication observed across these cancer cell lines was reduced and genomic ssDNA gaps were enhanced, consistent with the notion that TLS is required for replication during stress in these cancer cell lines. See, FIG. 36A and FIGS. 37B & 37C. Notably, upon TLSi treatment, MCF7 cells also showed a flattened morphology suggestive of senescence whereas HeLa cells halted replication and induced ssDNA even in the absence of HU treatment. See, FIG. 36A and FIG. 37B. These data suggest that TLS may also serve as a default mechanism for replication in some cancer cells

Thus, TLS provides a mechanism for cancer cells to replicate without global ssDNA gap induction when replication is confronted by exogenous or intrinsic stress. If TLS is fundamental for replication stress suppression in cancer, then TLSi could interfere with cell survival. To test this idea, clonogenic survival assays were performed in the presence and absence of a TLSi. The U2OS, RPE, human mammary epithelial cell line (HMEC), and patient fibroblast cell lines (e.g., FA2819 and PD846F) were unaffected by a TLSi. See, FIG. 36B and FIG. 37E. However, the cancer cell lines showing TLS-dependent replication dramatically lost their clonogenic capacity upon treatment with a TLSi. See, FIG. 36B.

To test the effect of a TLSi in an ex-vivo natural cancer model, early passage ovarian cancer ascites cells from two patient samples were analyzed for their ability to form colonies. Similar to the other cancer cell lines, a TLSi limited the overall survival of the ovarian cancer ascites cells, suggesting that TLS is indeed essential for their fitness. See, FIG. 36C. Taken together, these findings indicate that distinct cancer cell lines rely on TLS for replication without ssDNA gap induction and for overall cellular fitness.

I. Summary

The data presented herein demonstrates that replication stress response(s) are a robust protective barrier that arrests or eradicates cells that are undergoing replication stress such as from oncogene expression. Thus, cancers that overcome this barrier are expected to suppress the stress response by some mechanism such as by inactivation of tumor suppressors and/or checkpoint responses. However, tumor suppressors and checkpoint signaling pathways also suppress replication stress making it unclear how their loss could propel tumorigenesis. Based on the present disclosure, it is predicted that to overcome a replication stress response cells engage (e.g., activate) TLS.

Although it is not necessary to understand the mechanism of an invention, it is believed that distinct cancer cells rely on TLS to not only promote replication during extrinsic or intrinsic stress, but also to maintain cellular fitness. Mechanistically, it is found herein that, similar to ATR inactivation, TLS curtails the slowing and remodeling of replication forks and the global replication arrest response. However, unlike ATR inhibition, TLS also suppresses ssDNA gap induction that are currently believed toxic to cells. Forment et al., “Targeting the replication stress response in cancer” Pharmacol Ther 188:155-167 (2018); Gagna et al., “Use of anti-single stranded DNA antibodies to localize and quantify denatured DNA during cell death” Cell Biol Int 24:657-659 (2000); Peitsch et al., “DNA fragmentation during apoptosis is caused by frequent single-strand cuts” Nucleic Acids Res 21:4206-4209 (1993); Naruse et al., “Antibody against single-stranded DNA detects both programmed cell death and drug-induced apoptosis” Histochemistry 101:73-78 (1994); Chen et al., “Early detection of DNA strand breaks in the brain after transient focal ischemia: implications for the role of DNA damage in apoptosis and neuronal cell death” J Neurochem 69:232-245 (1997); Nur et al., “Single-stranded DNA induces ataxia telangiectasia mutant (ATM)/p53-dependent DNA damage and apoptotic signals” J Biol Chem 278:12475-12481 (2003); Huang et al., “Sensitivity and selectivity of the DNA damage sensor responsible for activating p53-dependent G1 arrest” Proc Natl Acad Sci USA 93:4827-4832 (1996); and Tidd et al., “Oligodeoxynucleotide 5mers containing a 5′-CpG induce apoptosis through a mitochondrial mechanism in T lymphocytic leukaemia cells” Nucleic Acids Res 28:2242-2250 (2000).

Thus, some embodiments of the present invention not only target TLS (e.g., with a TLSi) to enhance cancer therapy response, but also contemplate that TLS represents a vulnerable pathway for cancers in general.

Rewired replication that favors TLS could represent an adaptation to blunt oncogene induced replication stress that otherwise induces gaps, arrests cells or induces senescence. To facilitate continued replication during stress without ssDNA gaps, TLS polymerases could replicate despite low nucleotide pools, a condition that causes replicative polymerases to pause or skip over difficult to replicate regions, such as common fragile sites in early or late-replicating regions of the genome. Barnes et al., “Maintenance of Genome Integrity: How Mammalian Cells Orchestrate Genome Duplication by Coordinating Replicative and Specialized DNA Polymerases” Genes (Basel) 8 (2017). This skipping in the form of polymerase re-priming downstream of replication blocks could cause ssDNA gaps and reduced cell fitness.

FANCJ could promote TLS by disrupting secondary DNA fork structures, fork remodeling factors, or nucleases that interfere with TLS and in turn by stabilizing G-quadruplex secondary structures that are a substrate for REV1. Brosh et al., “Molecular and cellular functions of the FANCJ DNA helicase defective in cancer and in Fanconi anemia” Frontiers In Genetics 5:372 (2014); and Wu et al., “G-quadruplex recognition and remodeling by the FANCJ helicase” Nucleic Acids Res 44:8742-8753 (2016). Consistent with these functions, FANCJ^{S990A} enriches the chromatin association of REV1 and polh, and suppresses the nuclease MRE11 and the fork reversal and stability factor, RAD51. TLS is likely further licensed because FANCJ suppresses p21 that restricts TLS.

Conceivably, p21 suppression alone, as in cancer stem cells, suffices to avoid ssDNA-associated senescence. McGrail et al., “Defective Replication Stress Response Is Inherently Linked to the Cancer Stem Cell Phenotype” Cell Rep 23, 2095-2106 (2018). Other Fanconi anemia genes may function similar to FANCJ in TLS, given that FANCD2 loss also elevates p21 and reduces proliferation capacity. Ceccaldi et al., “Bone marrow failure in fanconi anemia is triggered by an exacerbated p53/p21 DNA damage response that impairs hematopoietic stem and progenitor cells” Cell Stem Cell 11:36-49 (2012). Although it is not necessary to understand the mechanism of an invention, it is believed that ssDNA gaps play a role in senescence and aging given that ssDNA gap suppression by Exol deletion improves the lifespan of telomere dysfunctional mice. Schaetzlein et al., “Exonuclease-1 deletion impairs DNA damage signaling and prolongs lifespan of telomere-dysfunctional mice” Cell 130: 863-877 (2007); and Pilzecker et al., “DNA damage tolerance in hematopoietic stem and progenitor cells in mice” Proc Natl Acad Sci USA 114:E6875-E6883 (2017).

While the full identity of TLS dependent cancer cells remains to be determined, a good place to start is with cancers expressing oncogenes that induce replication stress such as Cyclin E, CDC25A, KRAS, MOS, and MYC. Kotsantis et al., “Mechanisms of Oncogene-Induced Replication Stress: Jigsaw Falling into Place” Cancer Discov 8:537-555 (2018). Cancer cells can induce TLS in a few ways including, but not limited to, overexpression of TLS pathway components, enhanced TLS polymerase activities, or enrichment of factors promoting TLS. Notably, PARP10 is upregulated in human tumors and engages TLS to alleviate replication stress and promote tumorigenesis. Pilie et al., “State-of-the-art strategies for targeting the DNA damage response in cancer” Nat Rev Clin Oncol 16:81-104 (2019): and Schleicher et al., “PARP10 promotes cellular proliferation and tumorigenesis by alleviating replication stress” Nucleic Acids Res 46:8908-8916 (2018). It may also be possible to identify cancers dependent on TLS, by mutational signatures, expression of pro-TLS factors, or via their gene dependencies (see, for example, the cancer cell line encyclopedia; CCLE). It may be determined if cancers with a high FANCJ copy number displays a TLS signature. Gupta et al., “BRIP1 overexpression is correlated with clinical features and survival outcome of luminal breast cancer subtypes” Endocr Connect 7:65-77 (2018); Hampton et al., “A sequence-level map of chromosomal breakpoints in the MCF-7 breast cancer cell line yields insights into the evolution of a cancer genome” Genome Res 19:167-177 (2009); and Cantor et al., “Fork Protection and Therapy Resistance in Hereditary Breast Cancer” Cold Spring Harb Symp Quant Biol. (2018). Given that ssDNA gaps provide access to other mutagenic pathways it is possible that, in some genetic backgrounds, TLS suppresses mutagenesis.

It is demonstrated herein that not only do cancer cells rely on TLS for therapy resistance but also for basic cell fitness. These findings highlight that TLS inhibition alone could be a highly specific therapy for a wide-range of cancers.

III. Identification of Cancer Cells Vulnerable to TLS Inhibition

In one embodiment, the present invention contemplates a method comprising treating TLS-dependent cancers with TLS inhibitors. In one embodiment, TLS-dependent cancers are detected with TLS inhibitors. In one embodiment, the treating of TLS-dependent cancers with TLS inhibitors is lethal to TLS-dependent cancers.

Although it is not necessary to understand the mechanism of an invention, it is believed that TLS dependent cancers can be identified and selectively killed with TLS inhibitors (e.g., by inducing apoptosis). The data presented herein screens for cancer cell-TLS dependence and identifies TLS genes by analyzing patient outcomes and cancer cell viability based on gene dependence or expression in the latest cancer databases. The screens of available cancer cell collections for TLS dependence are based on TLS inhibition disrupting replication and clonogenic capacity. Comparisons are also made to establish whether non-transformed immortalized cells are not dependent on TLS. In one embodiment, the present invention contemplates a composition comprising at least one TLS inhibitor (TLSi) that is a broad and selective cancer therapy.

A. TLS Inhibition Reduces Replication and Clonogenic Capacity

The data presented herein suggest that in order to overcome a replication stress response, TLS is activated and promotes tumorigenesis due to acquired chemoresistance. For example, cancer cells can ignore exogenous stress induced by low dose HU treatment as found for U2OS cells with FANCJ^{S990A}. Indeed, similar to U2OS cells, RPE cells were arrested by HU, and replication continued in several other cancer cell lines such as in the breast cancer cell line, MCF7, the endometrial cancer cell line, HeLa and in the colon cancer cell line, HCT15. Moreover, this unrestrained replication with HU treatment was TLS-dependent as evidenced by the data herein showing that TLSi 1 reduced replication and induced ssDNA gap induction. Notably, TLSi 1 reduced replication and induced ssDNA gaps in the HeLa cells even in the absence of HU, suggesting that TLS is involved in the suppression of intrinsic replication stress in HeLa cells. See, FIG. 9B and FIG. 9C. Further suggesting that cancer cells have high intrinsic replication stress that needs to be subverted in order to maintain fitness, all three TLS cancer cell lines, unlike non-TLS U2OS or RPE cells, lost clonogenic survival when treated with TLSi 1 with no additional stress added. See, FIG. 7D. Together, these findings suggest that TLSi could act as a monotherapy to treat a broad spectrum of tumors.

B. Identification of TLS Cancer Cells

Screening for synthetic lethal genes involved in cancer cell TLS dependence was performed using the Cancer Cell Line Encyclopedia (CCLE). The FANCJ gene, which is a tumor suppressor, and is often mutated to cause loss-of-function was screened. For cancers dependent on FANCJ, it was expected that FANCJ would be either over-expressed or mutated to cause a gain-of-function similar to FANCJ^{S990A} that increases replication rates during stress. Indeed, several cancers were found both dependent on FANCJ and over-expressed, including MCF7 cells⁷⁵⁻⁷⁸. depmap.org/portal/gene/BRIP1?tab=dependency.

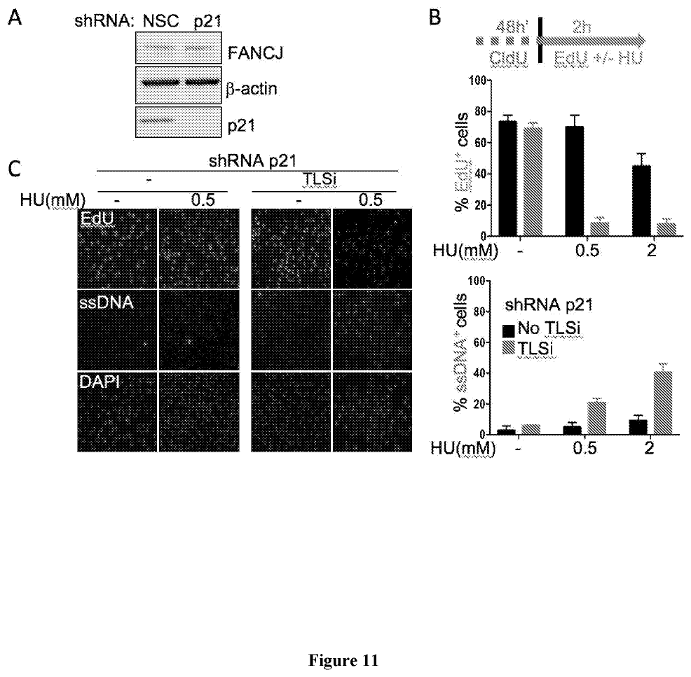

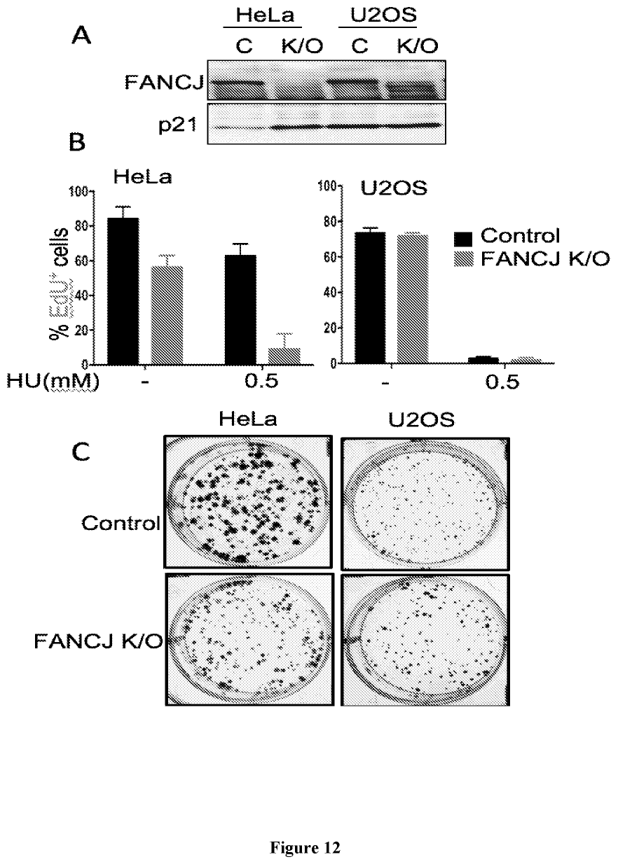

Using CRISPR-CAS9, FANCJ^{K/O} HeLa and U2OS cells were generated, however MCF7 cells were not viable, perhaps because FANCJ is essential or the high copy numbers lead to toxic CRISPR-CAS9 induced breaks that reduce viability. Notably, as compared to FANCJ^{K/O} in U2OS, in FANCJ^{K/O} in HeLa cells had higher p21 levels and both reduced replication in HU and clonogenic capacity in unchallenged conditions. See, FIG. 12. These findings further indicate that FANCJ is a pro-TLS factor and that loss of TLS by gene K/O or inhibition will disrupt replication in TLS, but not in non-TLS cancer cells.

These findings indicate that TLSi 1 could be used to screen cancer cells for TLS dependence. Thus, available cancer cell collections can be screened to determine if the deposited cell lines are TLS or non-TLS cells. As controls, known TLS cancer cells (e.g., HeLa, MCF7) and non-TLS cancer or non-cancer cells (e.g., U2OS vs RPE, respectively) can be utilized. The screen can also identify non-cancer immortalized cell lines to determine whether TLSi exclusively limits the fitness of cancer cell lines. In addition to RPE cells, approximately ten non-cancer immortalized cell lines have now been identified that can be analyzed for replication and clonogenic capacity with and without TLSi 1 to address whether TLSi will be selective to cancer. Other cell culture screens can utilize cancer cell line panels such as the NCI-60 cancer cell line set. Cancer cell lines can be selected from the CCLE database by having a predicted dependence on TLS genes vs non-dependent lines. Following the identification of TLS vs non-TLS cancer cells, the screens can be expanded to assess cell viability with the Broad PRISM cell line. Approximately seven hundred and fifty (750) cell lines have been extensively characterized and can provide the opportunity to assign commonalities and distinctions between TLS vs non-TLS cells that could uncover biomarkers of TLS and insight into how TLS is activated.

To develop a TLS DNA signature predictive of TLS cancer cells and therefore vulnerable to TLSi treatment, one should identify events that are common to the proteome and genomic patterns of TLS cancers and therefore distinct from non-TLS cancer. For example, differences in mutation rates or other genomic alterations that could be amenable as biomarkers. Continued replication despite HU treatment could induce mutations when error-prone TLS is employed.

Alternatively, TLS could protect the genome from mutagenesis that initiates on ssDNA gaps. In cells with TLS cancer, it can be determined that the frequency of mutations or chromosomal aberrations following replication stress can be bypassed (e.g., suggesting DNA crosslinks) or cannot be bypassed (e.g., suggesting DNA breaks) readily. For example, TLS cells with FANCJ^{S990A} were previously shown to be hyper-resistant to UV and MMC that induce DNA crosslinks, but sensitive to camptothecin (CPT) that induces breaks^{14,32}. Assessment of mutation frequency following these agents can easily be made using the hypoxanthine phosphoribosyl transferase (HPRT) gene. Using this HPRT assay, a 10-fold increase in the UV-induced mutation frequency in FANCJ deficient cells has been reported³². Chromosomal aberrations can also be assessed as indicators^{33,37}.

Genomic DNA can be prepared to perform WGS from starting single cell clones, vs clones left untreated or treated with low dose sublethal of replication stress (such as HU treatment) for 24 h and subsequently propagated for ˜50 days (e.g., ˜100 cell divisions). One observation is whether stress-activated TLS dependent cells (e.g., S990A mutant U2OS) will have greater mutations than TLS-independent (WT U2OS cells). If mutations are elevated, TLSi 1 may suppress the effect of these mutations. Mutation frequency, with or without stress in TLS-stress activated cancer cells, such as MCF7, may be suppressed by TLSi 1. By applying WGS to a range of TLS activated or inhibited cell lines, it can be determined if TLS contributes to mutagenesis in a cell context dependent manner.

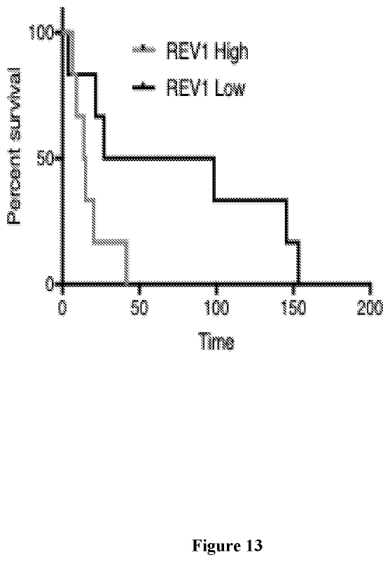

Identification of TLS cancer cell lines is expected by screening for gene-dependencies and by non-biased screening of large cancer cell collections. Cancer cell collections have been screened and analyzed for many parameters (transcriptomes, proteomes etc.) providing a wealth of information that could provide insight towards TLS biomarkers and mechanisms of activation. For example, the screens may find selective TLS targets from proteomics and genes predicting poor patient outcomes. Indeed, BRCA2 deficient cancers show TLS dependence as a predictor of poor patient outcomes³³. See, FIG. 13. Aside from identifying TLS cancers with a TLS inhibitor, loss of fitness could be screened upon K/O of TLS genes. This concept is supported by the finding that several cancer lines have high FANCJ copy number.

High REV1 mRNA predicts a poor response in BRCA2-deficient ovarian cancer. TCGA ovarian cancer patients with germline mutations that deactivate BRCA2 (e.g. truncated by premature stop codon) were identified using the Gnome Analysis Tool Kit, and plotted survival in cBioPortal for high and low REV1 mRNA levels (top 25% vs bottom 25%). to MCF775-78 that show loss of viable without FANCJ (i.e. FANCJ K/O in MCF7 was not successful). Cancer cell collection screens could be extended to cancers expressing oncogenes that induce replication stress such as Cyclin E, CDC25A, KRAS, MOS, and MYC81. Lastly, it may also be possible to identify cancers dependent on TLS, by mutational signatures. Failure to slow replication due to TLS could have consequences for genomic stability. Indeed, the FANCD2-FAN1 interaction is not required for MMC resistance, but is required to slow replication following HU treatment and to maintain genome integrity following treatment with HU or MMC⁷¹. Thus, eliminating TLS function in cancer could also reduce the ability of the cancer to mutate, and therefore retain its vulnerability to other therapies. While TLS depletion restricts cisplatin resistance, it is not clear if TLS disruption can stop cancer from forming^{82,83}.

In one embodiment, the present invention contemplates a method as to whether TLSi and/or TLS biomarkers have clinical or predictive value. Ideally, TLS inhibition with a small molecule disrupts cancer initiation as the TLS machinery is disassembled limiting cancer cell adaptation.

C. BRCA Cell Chemoresistance and TLS

It has previously been reported that TLS may be a mechanism in cancer that undermines therapy response. Specifically, CHD4 was identified in an unbiased screen as a gene whose loss conferred cisplatin and PARPi resistance in BRCA2 deficient ovarian cancer cells³³. CHD4-loss also occurs de novo in chemoresistant BRCA2 cancer and elevates TLS. Other genes have since been identified such as EZH2 or FEN1, however CHD4 loss has the strongest chemoresistance phenotype in cell culture and prediction power in patient databases^{33,79,80}.

In particular, the TCGA dataset presents three hundred and twelve (312) high-grade serous ovarian cancers for which gene expression, DNA copy number, promoter methylation and whole-exome DNA sequencing data are available. Evaluation of the association of expression of CHD4 with overall survival (OS) and progression free survival (PFS). Using the Affymetrix U133 raw gene expression database a survival analysis was performed using BRB-ArrayTools Version 4.2 (Biometrics Research Branch, National Cancer Institute, Bethesda, MD). In BRCA2 tumors, those cancers with an overexpressed CHD4 (i.e. those with higher than median expression of CHD4) had a better PFS and OS compared to the remaining tumors (i.e. those with lower than median expression of CHD4). Given these findings that CHD4 loss activates TLS, the data suggest that TLS gap-filling promotes chemoresistance in BRCA cancer.

D. Patient Outcome Prediction and TLS Genes

In one embodiment, the present invention contemplates a method comprising predicting patient survival in BRCA2 deficient cancer by evaluating TLS gene function. In preliminary findings, a high REV1 correlated with a poor patient response in BRCA2 deficient ovarian cancer. See, FIG. 13. These results clearly demonstrate the clinical relevance of REV1 as a cancer target and highlight the significance of targeting REV1-mediated TLS as a novel therapeutic strategy. Conversely, it would be expected that a poor patient response correlates with low levels of negative regulators of TLS, such as p21.

### IV. Identification of TLS Inhibitors

In one embodiment, the present invention contemplates a composition comprising a TLS inhibitor (TLSi). In one embodiment, the present invention contemplates a method to identify a

TLS inhibitor with enhanced potency and improved drug-like properties for development as anti-cancer agents. In one embodiment, the present invention contemplates a method comprising treating a cancer patient with a TLS inhibitor such that the cancer cells are killed.

As discussed above, the present invention discloses that small molecule inhibition of TLS is an effective cancer chemotherapeutic strategy. The data disclosed herein shows that REV1 targeting compounds selectively inhibit DNA replication and clonogenic capacity exclusively in

TLS-dependent cancer cells. Similar to the data regarding TLSi 1, this strategy leads to an ability to prioritize lead inhibitor compound scaffolds. These scaffolds are then derivatized to assess potency and drug-like properties to identify structure-activity relationships that maximize cancer therapy efficacy.

A. REV1 C-Terminus Targeted Compounds

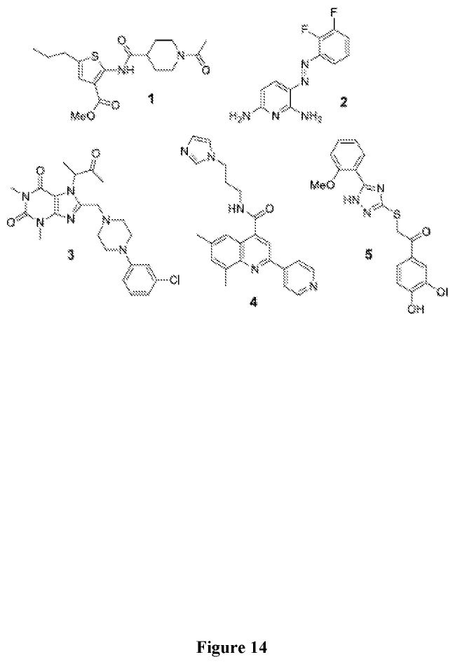

Biochemical and computational lead identification techniques were used to identify multiple, distinct small molecule scaffolds that disrupt the protein-protein interaction (PPI) between the C-terminal domain of REV1 (Rev1-CT) and the Revl interacting region (RIR) of several other TLS polymerases^{39,43}. See, FIG. 14. In vitro studies demonstrate that two of these compounds (TLSi 1 and TLSi 2) curtail replication stress avoidance in TLS cancer cells. See, FIG. 15.

Moreover, preliminary findings also show that TLSi 1 reduces clonogenic capacity of TLS cancer cells, but has little effect on non-TLS cells. See, FIG. 9D. Collectively, these findings suggest that replication ssDNA gaps are a biomarker of cancer therapy efficacy. Furthermore, agents that restore replication ssDNA gaps (e.g., REV1 inhibitors) may provide a therapeutic strategy for a variety of human malignancies.

These REV1 targeted compounds have been able to probe the role of TLS in the replication stress response in cell culture models; however, it remains to be determined whether these compounds have the properties necessary to be effective as cancer therapies. Nonetheless, the compounds may be prioritized according to activity selective to TLS cancer for development of chemical scaffolds as TLS inhibitors and anti-cancer agents through a series of preclinical in vitro studies to prepare optimized compounds for future in vivo models. In order to determine which of our lead scaffold(s) holds the most promise for further development, selected compounds can be evaluated in a series of preclinical in vitro studies to establish selectivity to TLS cancer, potency and drug-like parameters to establish selectivity to TLS cancer, potency and drug-like parameters.

As described above, multiple cellular system has been characterized to study the ability of REV1 inhibitors to selectively inhibit TLS cancer but not non-TLS cancer or non-cancer cells. For example, in non-TLS control cells (U2OS/RPE) these compounds do not affect replication forks or DNA gaps. TLS is activated in cancer cells by the addition of an external stress (U2OS-FANCJ^{S990A}, U2OS p21 depletion, and MCF-7/HCT15). Finally, TLS is constitutively active in HeLa cells.

A combination assay was performed in a high-throughput manner using 384-well plates. This assay served as a rapid and efficient method to determine the anti-TLS activity of the lead compounds and novel analogues. See, FIG. 15. Specifically, the assay identifies compounds that cause TLS cells, but not non-TLS cells, to lose replication (green) and gain ssDNA gaps (red). See, FIG. 15B. Colors can be identified using a high content and high throughput imaging camera. Initial screening optimization studies indicate that red and green signals are readily detected compared to controls. Moreover, red and green fluorescent readouts are readable/quantifiable from full well images at 20×. Several positive and negative controls that may be employed in parallel with the screen to induce gaps or suppress gaps. In particular, under these assay conditions replication with gaps are induced when ATR is inhibited.

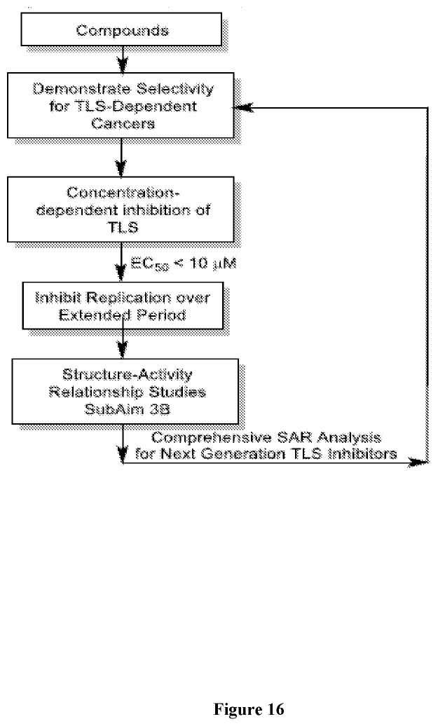

The lead compounds can be prioritized through potency and pharmacokinetic studies. On approach is to determine as to whether the compounds are selective to TLS cancer. For example, TLSi 3-5 may be selected to determining if their selectivity for TLS cancer is similar to TLSi 1 and 2. A combined assay can be performed to first analyze the ability of these TLSi's to inhibit global replication with concomitant induction of ssDNA in TLS cancer vs non-TLS cancer or non-cancer cells. Second, a colony formation assays in each of these models to determine whether the TLSi's selectively reduce colony formation in TLS cancer cells. See, FIG. 16. These TLSi's are then prioritized in accordance with selective activity in TLS cancers.

Initially, an ability of each TLSi to selectively inhibit TLS at a single concentration (e.g., 20 μM) can be determined. TLS specific compounds are re-evaluated in the combined and colony formation assays at multiple concentrations to determine EC₅₀ values for each selective TLS inhibitor. As noted, the combination assay measures TLS inhibition after a 2-hr incubation; therefore, compounds with an EC₅₀ less than 10 μM are further evaluated under extended incubation periods (in hour to day increments) to determine if they are durable enough in cell culture to inhibit replication over a prolonged period. In addition to utilizing this series of cellular experiments to prioritize REV1 compounds, binding affinities (K_{d}) for each compound are measured in a complex with Revl-CT via surface plasmon resonance (SPR). Baseline solubility (pH 7.4) and stability (T1/2, human liver microsomes) parameters are also established for these compounds. Taken together, these studies provide a complete characterization for each the lead compounds. See, Table 1.

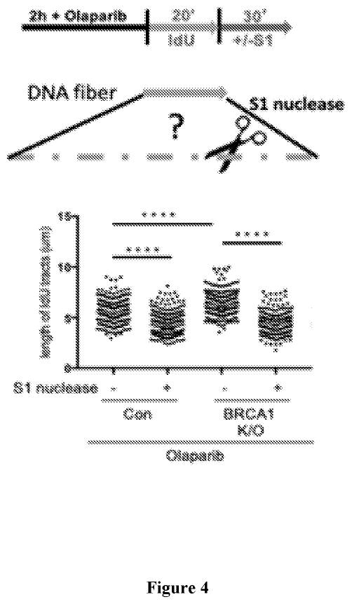

TABLE 1
Preliminary potency and in vitro PK parameters for lead TLSi's.

| | | Colony | | | | |
|---|---|---|---|---|---|---|
| | | Formation | | | | T_{1/2} |
| Cmpd | TLS specificity | Assay | Durability | K_{d} | Solubility | (min) |
| 1 | Yes (FIG. 4B) | Yes | — | — | 63.3 μM | 36.9 |
| 2 | Yes (FIG. 11B) | — | — | — | 8.5 μM | <2.3 |
| 3 | — | — | — | — | 176.6 | 3.4 |
| 4 | — | — | — | — | 146.1 | 4.7 |
| 5 | — | — | — | — | — | — |

Collectively, this data prioritizes REV1 inhibitor scaffolds based on a holistic approach to compound optimization prior to further structure-activity relationship studies.

After prioritizing the lead scaffolds, designing improved analogues utilizes ligand, structure, and computational approaches to guide conservative and systematic modifications to distinct regions of the scaffold to explore structure-activity relationships (SAR) for these compounds. In particular, analogues of the most promising compounds are prepared using rational design principles of medicinal chemistry. As a representative example, a series of analogues of compound 2 in which the azo linker between the phenyl and pyridine rings is modified will be prepared and evaluated. Multiple clinically used prodrugs contain an azo linker that is cleaved by bacterial azoreductases in the gut microflora to release the active form of the drug and a stability analysis also demonstrates that compound 2 is not stable enough for in vivo administration (Table 1, T1/2<2.3 min)⁸⁴. Consequently, the azo linker in the phenazopyridine scaffold may be a metabolic liability.

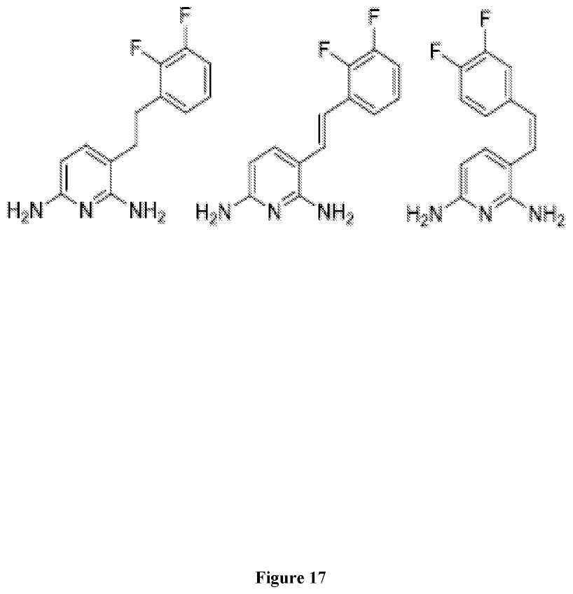

The azo group was then replaced with several chemically distinct tethers to determine not only whether these modifications improve overall scaffold stability, but also whether these changes affect TLS inhibition. Analogues can be prepared that incorporate unsaturated or saturated linkers as direct corollaries to the azo linker. See, FIG. 17. These analogue compounds allow a determination as to whether compound flexibility affects TLS inhibition. In addition, analogues are prepared that extend or reduce a tether link to explore the optimal distance between the two aromatic moieties.

The overall results from all of these assays are integrated to design subsequent analogue generations with improved potency and drug-like properties. At any point during this process, the scaffold can be subjected to further structure-activity relationship studies to improve potency and/or binding affinity or address PK liabilities.

Lead scaffolds demonstrate TLS inhibition and cellular potency that substantiate their advancement into the medicinal chemistry optimization phase of drug development. Exhibition of potent inhibition of TLS and significant cell killing effects, justifies the evaluation of analogues at lower concentrations. The benchmarks that have been set for advancing compounds to the next phase are primarily based on the activity of compound 1. However, these benchmark metrics may be modified to ensure that an appropriate number of analogues are advanced to the next stage.

V. Cancer Therapy and DNA Gap Induction In one embodiment, the present invention contemplates a method comprising at least one chemotherapy comprising sensitizing BRCA-deficient cancer to elicit an anti-cancer effect by inducing single stranded DNA (ssDNA) gaps in replicating DNA. See, FIG. 5. In one embodiment, the method does not target defects in HR or FP. The data presented herein demonstrates that, in response to replication stress, BRCA-RAD51 deficient cells fail to effectively restrain DNA replication and ssDNA gaps develop. Moreover, BRCA deficient cancer cells are sensitive to drugs, such as poly(ADP) ribose polymerase, and PARP inhibitors (PARPi) such that ssDNA gaps are exacerbated in BRCA deficient cells. Furthermore, ssDNA gap suppression confers chemoresistance in tissue culture and in cancer patients even when HR or FP are defective. Although it is not necessary to understand the mechanism of an invention, it is believed that BRCA1 deficiency is most effectively treated by cancer therapies that induce ssDNA gaps, such that gap formation is a biomarker of a tumor response to a chemotherapeutic agent. It is further believed that, to maximize therapy response, pathways limiting ssDNA gap formation should be targeted (e.g., inhibited) to prevent ssDNA gaps from being filled such that cancer therapies concomitantly become more efficacious.

The data presented herein demonstrate quantitative isolation of proteins at replication forks in parallel with visualization and quantification of DNA replication intermediates in DNA fiber assays performed in a series of knockout (k/o) human fibroblast and cancer cell lines generated by CRISPR gene-editing technology. These data demonstrate that: (1) the BRCA1/2-RAD51 pathway slows the DNA replication rate during stress to limit ssDNA gap induction; (2) a replication stress-inducing agent (e.g., PARPi) induces ssDNA gap formation resulting in an accumulation of ssDNA gaps which are toxic to a cancer cell; and (3) suppression of ssDNA gap induction mediates chemoresistance (e.g., cancer therapy failure). Although it is not necessary to understand the mechanism of an invention, it is believed that by uncovering molecular transactions at replication forks that determine cancer cell response to therapy, the term “BRCA deficiency” (e.g., :BRCAness”) can be redefined to describe how therapies sensitize cancer cells thereby defining compositions and methods for targeting resistant pathways and identifying biomarkers of clinically-relevant chemotherapeutic response.

A. Conventional Approaches to Cancer Therapy

It is currently believed in the art that loss of the breast cancer susceptibility genes (BRCA1 or BRCA2) in hereditary breast and ovarian cancer (HBOC) is characterized by defects in the protection of replication forks. This is commonly referred to as replication fork protection

(FP)) where DNA repair proceeds by homologous recombination (HR). It is commonly thought that HR and FP deficiencies produce points of vulnerability in cancer cells that sensitize them to DNA damaging agents such as to cisplatin and PARPi. However, it has recently been suggested that these replication stress inducing agents do not arrest or even initially pause replication forks, instead, chemotherapeutics (e.g., PARPi) accelerate replication.¹⁻³.

Thus, breaks or stalled replication fork structures may not be lesions that result in BRCA deficient sensitivity. This raises the question as to what really is the lesion that causes synthetic lethality to PARPi? This knowledge gap limits any ability to treat HBOC or understand how the BRCA-pathway is bypassed in chemotherapy resistant cancer which is believed to be a substantial obstacle in the long-term and effective treatment of BRCA1/2 cancers.

Given the involvement of HR proteins in double strand break (DSB) repair, the function of HR factors at replication forks was initially thought to relate to fixing DSBs at collapsed forks³⁵. However, growing evidence suggests a DSB repair-independent role for HR factors at the replication fork. HR factors have been detected on chromatin during unperturbed replication and are recruited to stalled replication forks independent of DSB formation³⁶⁻³⁸. Upon replication fork stalling, HR factors prevent excessive nucleolytic degradation of nascent DNA strands by the MRE11 nuclease, and this FP function is genetically uncoupled from DSB repair³⁸⁻⁴⁰. Reversed replication forks are degraded in BRCA deficient cells. Thus, FP is restored by loss of replication fork remodeler genes, such as SMARCAL1 or RAD51, that assists in replication fork reversa1^{38,41-43}.

Collectively, these studies demonstrate a role for the BRCA-RAD51 pathway beyond HR at a replication fork⁴⁴. While not well studied, BRCA-RAD51 proteins also function prior to replication fork stalling in the immediate response to replication stress. For example, RAD51 has been reported to function by restraining replication and therefore prevents replication ssDNA gaps during replication stress^{38,45}. Studies in frog extracts demonstrate that by promoting Rad51 binding to replicating DNA, BRCA2 also prevents ssDNA gap accumulation^{38,42}. Although it is not necessary to understand the mechanism of an invention, it is believed that in human cells BRCA functions with RAD51 in an initial response to replication stress to slow (e.g., restrain) replication and limit ssDNA gap induction. It is further believed that this function is distinct from FP, or HR and contributes to the efficacy of cancer therapy outcomes.

In contrast to the above, some embodiments of the present invention analyze DNA replication fork dynamics and cellular responses to identify alternative determinants of cancer therapy response and chemoresistance. These alternative pathways may promote or undermine therapy response in cancer. In particular, the role of discontinuous replication due to the termination and restart of replication vs continuous replication that avoids gaps is a considered to be involved. The model system of BRCA deficient cancer is utilized below to exemplify these alternative pathways. However, in no way is the present data limited in scope to BRCA-related cancers, as it is believed that the TLS mechanisms described above are common to all cancer types and are expected to be equally sensitive to related chemotherapy as well as avoiding the development of chemoresistance. One proposed unified mechanism of resistance provides biomarkers of response “BRCAness” (i.e. ssDNA gaps) and focuses towards defining and inhibiting pathways of resistance (i.e. gap suppression pathways).

B. Cancer Therapeutics by Regulating DNA Replication Stress

In one embodiment, the present invention contemplates a method to specifically kill cancer cells by uncovering and targeting survival pathways that subvert the replication stress response. In one embodiment, the method uncovers how a replication stress response is subverted in cancer and develops compositions to block these subversion pathways. Although it is not necessary to understand the mechanism of an invention, it is believed that a replication stress response limits cancer progression and preserves genome integrity, and accordingly cancer cells can subvert the response to survive. Indeed, similar to replication perturbations, it has been observed that oncogenes activate the replication stress response which can induce replicative senescence. Thus, the replication stress response may provide a barrier against malignant transformation. Therefore, subversion of the replication stress response may be involved in tumorigenesis and therefore could be targeted to reduce and/or prevent cancer.

In one embodiment, the present invention contemplates a method for predicting cancer therapy failure by determining the frequency of ssDNA gaps present in a cancerous tissue. Such gaps are indicative of a failure of DNA repair mechanisms. In one embodiment, the method predicts that cancer cells comprising DNA having a high frequency of ssDNA gaps are susceptible to cancer therapies. In one embodiment, the method predicts that cancer cells comprising DNA having a low frequency of ssDNA gaps are not susceptible (e.g., chemoresistant) to cancer therapies.

In one embodiment, the present invention contemplates a method to predict chemotherapeutic efficacy in ovarian cancer patients. For example, the method identifies vulnerabilities in ovarian tumors with mutations in the hereditary breast cancer genes. It has been found that ovarian cancer cells deficient in the BRCA genes fail to properly respond to stress such as induced by chemotherapy. Consequently, the process of duplicating the cells' DNA, called DNA replication, aberrantly continues. The data presented herein shows that this continued DNA replication during chemotherapy stress leads to an anemic DNA product. Rather than being the expected DNA double helix, the DNA has regions of single stranded DNA, “replication gaps”. Although it is not necessary to understand the mechanism of an invention, it is believed that when ssDNA gaps are present, BRCA deficient ovarian cancer cells are sensitive to therapy and when gaps are avoided, resistance occurs. The present findings that ssDNA gaps are involved in chemotherapic responses of ovarian cancers is a paradigm shift in the current framework that proposes that persistent DNA breaks and fork degradation is the cause of sensitivity. Thus, it is herein proposed to determine the molecular determinants of this BRCA pathway fork restraint function. Moreover, the data identifies the gap making machinery that may be involved in therapy response and the gap avoidance machinery that provides therapy resistance. Current chemotherapy resistance theories previously attributed to restored DNA repair and fork protection are compared to determine if ssDNA gap suppression is instead the fundamental resistance mechanism. Collectively, these data identify how ovarian cancer cells succumb to and eventually gain resistance to chemotherapy and provide valuable insight towards biomarkers predicting resistance and drugs that prevent resistance.

Although it is not necessary to understand the mechanism of an invention, it is believed that subversion of replication stress response involves TLS. This premise is based on the above disclosed data and previous reports demonstrating that TLS functions during replication and is activated in response to stress¹⁸. TLS loss may lead to delayed progression through S phase, slower replication fork extension rates, stalled and/or collapsed replication forks and genomic instability. Each of these events have been reported to be exacerbated by replication stress^{19,20}. Under the current working hypothesis, TLS promotes replication during replication stress; however, molecular details explaining the phenomenon are lacking. The data presented herein suggests that these events may be explained by altered DNA replication fork dynamics in cells with TLS activated through expression of a pro-TLS FANCJ mutant. See, FIGS. 6-8.

The data described herein seeks to identify how TLS keeps replication forks from effectively slowing. For example, understanding the components of a proficient vs disrupted TLS replisome may provide evidence as to how TLS is involved in cancer and whether the pathway can be targeted. These data further show that TLS enables cancer cells to maintain fitness by replicating both during stress and in unstressed conditions and maintain clonogenic capacity. See, FIG. 9. These findings indicate that cancer cells use TLS not only to combat replication stress, as induced by chemotherapies such as cisplatin, but also for viability. As such, TLS inhibition may be a promising therapeutic target for a diverse set of cancers that co-opt TLS by different mechanisms. In one embodiment, the present invention contemplates compositions comprising TLS inhibitors for use in methods for treating cancer.

Although it is not necessary to understand the mechanism of an invention, it is believed that chemotherapeutic drugs including, but not limited to, cisplatin and/or PARPi do not function to prevent DNA double strand break induction as had been conventional in the art for decades. In one embodiment, the present invention contemplates a method for treating cancer comprising a chemotherapeutic drug that induces single stranded DNA (ssDNA) gaps. In one embodiment, the induced ssDNA gaps develop in a DNA replication fork.

The present invention thereby discloses that chemotherapeutic drugs are clinically effective on treating cancers, not because of defects in DNA repair or replication fork protection, but rather because chemosensitive cancers have a high level of intrinsic DNA replication ssDNA gaps. For example, BRCA-deficient cells develop ssDNA gaps in response to replication stress because they have defects in restraining or slowing DNA replication. It is known that wild-type BRCA in normal cells slow or pause replication rates to prevent ssDNA gap formation when under replication stress. It is believed that this slowing of replication rates creates a natural barrier against the development of cancer. However, because cancer cells may be deficient in BRCA1, BRCA2, and/or RAD51 gene function, such replication restraint regulation is lost and the cancer cell continues to replicate during stress in a discontinuous manner that leads to the accumulation of replication gaps (e.g., ssDNA segments). See, FIG. 1. Therefore, when a cancer cell is treated with a DNA replication stress-inducing agent such as cisplatin or PARPi, BRCA-deficient cancer cells develop a toxic accumulation of gaps that results in the death of the cancer cell. Although it is not necessary to understand the mechanism of an invention, it is believed that chemotherapeutic agents and/or drugs, and likely many other anti-cancer medications, induce single stranded gaps in cancer cell DNA as a result of replication stress.

In one embodiment, the present invention contemplates a method for predicting chemotherapeutic efficacy comprising contacting a cancer cell with a chemotherapeutic agent and detecting a plurality of single stranded gaps in a cancer cell DNA segment. The data presented herein reveal that ssDNA gap induction by chemotherapeutic agents is a predictor of cancer therapy efficacy. For example, cancer cells that are deficient in replication rate regulatory genes including, but not limited to, BRCA1, BRCA2, and RAD51, are sensitive to chemotherapy due to the accumulation of ssDNA gaps. On the other hand, cancer cells that are BRCA1/2 or RAD51 proficient cells are non-sensitive to chemotherapy and do not accumulate ssDNA gaps during chemotherapeutic-induced replication stress.

Consistent with this point, it has been found that BRCA-deficient cancers that also suppress ssDNA gap induction acquire chemoresistance to cancer therapy. One mechanism of ssDNA gap suppression may include restored replication fork restraint, which can be achieved by (for example) reversion mutations in the BRCA genes. In addition, BRCA-deficient cancer cells can gain therapy resistance via a gap filling mechanism that employs translesion synthesis. In this case, replication fork restraint is not restored and replication stress continues but ssDNA gaps do not accumulate because they are concomitantly repaired upon formation.

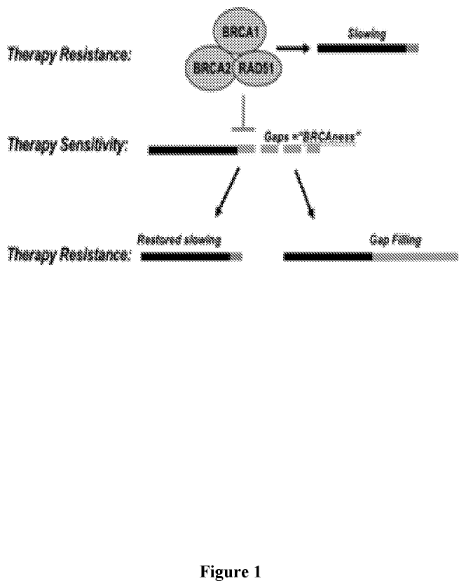

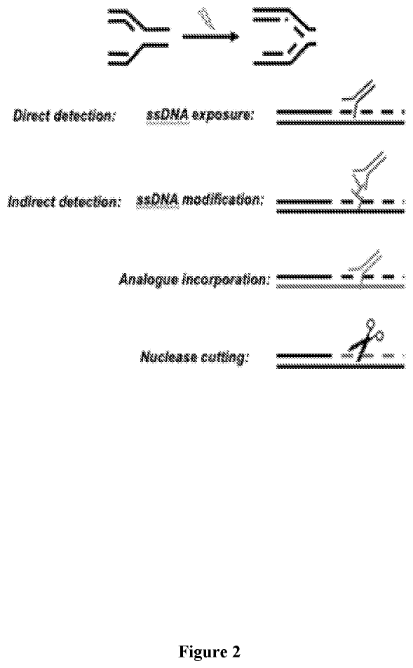

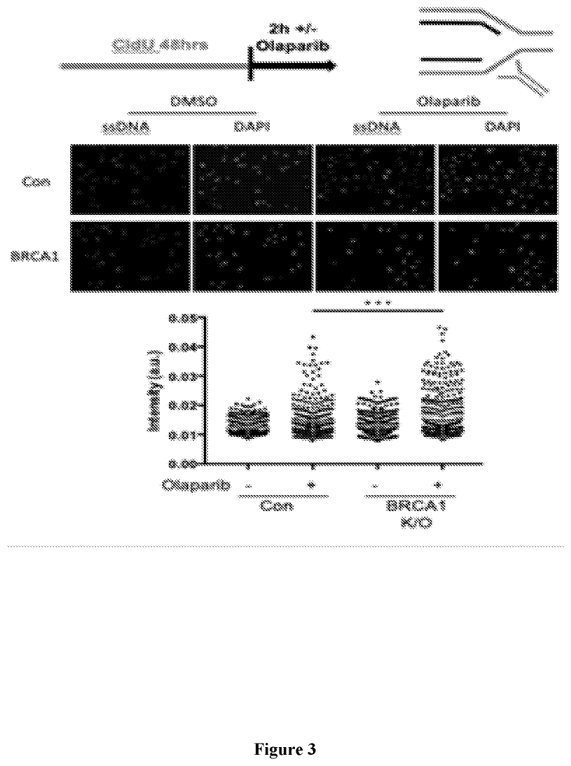

In one embodiment, the present invention contemplates a method for predicting chemotherapeutic efficacy comprising detecting ssDNA gaps in a cancer cell DNA segment with a biomarker or probe. In one embodiment, ssDNA gaps may be detected with several distinct reagents, including antibodies that directly detect single stranded DNA regions or that detect modifications uniquely occurring on ssDNA regions, such parylation or de-amination. Furthermore, ssDNA gaps may be detected via nucleotide analogue incorporation into the cancer cell genome. See, FIG. 2. When DNA analogues are incorporated into the genome, and non-denaturing immunofluorescence (IF) is performed, antibodies will only detect the analogue when present in a single stranded region. See, FIG. 3.

In one embodiment, ssDNA regions may be detected in replication forks by performing a single molecule DNA fiber analysis. For example, a linear nucleic acid segment is stretched on a microscope slide. By treatment with a nuclease that cuts ssDNA regions, the ssDNA gaps can be detected. If ssDNA gaps are present and concomitantly cut, the DNA fibers necessarily shrink as compared to a control. On the other hand, nucleic acid segments without ssDNA gaps will not be cut and will remain the same size. See, FIG. 4.

C. ssDNA Gap Avoidance by Replication Restraint

Replication fork restraint is believed to be functionally distinct from HR and/or FP. The data presented herein analyzes an ability of RAD51, BRCA1, and BRCA2 mutants with and without; i) HR; ii) FP; iii) complex formation; or iv) filament forming activity, to restrain replication during stress. In addition, data is presented which analyzes ssDNA gap formation kinetics in relation to checkpoint induction, fork degradation, and/or DNA breaks. These data show whether replication fork restraint and ssDNA gap avoidance plays a role in the BRCA pathway that protects the genome and whether BRCA activity requires RAD51 filament formation instead of the commonly believed pathway involving HR and FP.

1. BRCA1/2 Deficient Cancer Cells Fail to Properly Restrain Replication During Stress and Gaps Develop.

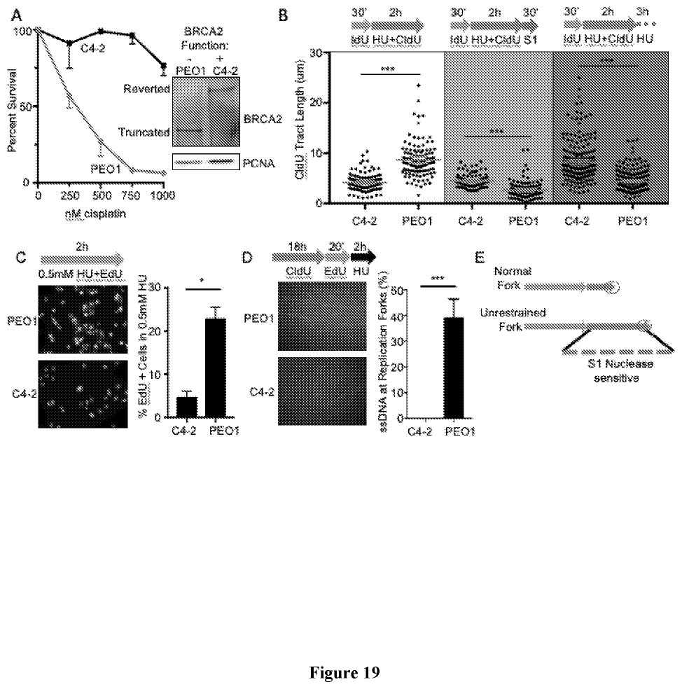

To address the role of BRCA2 in the initial stress response, studies were performed with the isogenic PEO1 parental ovarian cancer cell line with truncated BRCA2 and cisplatin hypersensitivity and the PEO1 reversion cell line, C4-2, with BRCA2 function and cisplatin resistance⁴⁶. See, FIG. 19A. The cell lines were incubated with 5-Iodo-2-deoxyuridine (IdU) for 30 minutes followed by 5-chloro-2′-deoxyuridine (CldU) for 2 h in the presence of hydroxyurea (HU). Longer CldU tracks were observed in PEO1 cells compared to C4-2 cells, indicating replication in PEO1 cells had continued during HU treatment. See, FIG. 19B. These data suggest that BRCA2 effectively restrains replication during stress. In agreement with the fiber assays, analysis of global cellular replication based on 5-ethynyl-2′-deoxyuridine (EdU) incorporation supported that BRCA2 restrains replication during stress. See, FIG. 19C.

The observed failure to fully restrain replication in response to stress in BRCA deficient cells might result in poorly replicated regions that contain ssDNA gaps. A DNA fiber assay was performed followed by incubation with S1 nuclease. S1 cuts at ssDNA regions and secondary DNA structures as an indicator of poor quality DNA⁴⁷. The data show that labelled nascent DNA tracks were S1 sensitive in PEO1 cells, but not in C4-2 cells. See, FIG. 19B. Suggesting that ssDNA gaps are throughout the region replicating during stress, DNA tracts in PEO1 cells were shortened to a length found in C4-2 cells in which replication stopped in response to stress. These S1 sensitive nascent DNA regions were degraded after continued exposure to replication stress. Next, the genome was labeled with CldU for 18 to 24 h and non-denaturing immunofluorescence (IF) was performed to detect regions of ssDNA. To determine whether ssDNA gaps were in the vicinity of an active replication fork, the cells were pulsed with EdU to define active replication by Click-it chemistry which can be visualized on DNA without denaturing. These data showed that ssDNA gaps were present in regions of active replication in BRCA2 deficient PEO1 cells, but not in BRCA2 proficient C4-2 cells. See, FIG. 19D. These data are consistent with the above S1 nuclease results. Thus, BRCA2 deficient PEO1 cells fail to fully restrain replication in the presence of stress, creating ssDNA gaps throughout the replicating region during stress that are degraded after additional exposure to stress. See, FIG. 19E.

2. Gap Suppression is Linked to Restored Replication Fork Slowing and Gap Filling by TLS

To understand the molecular mechanisms underlying the process of ssDNA gap formation in BRCA2 deficient cells, ssDNA gap suppression was investigated under conditions of chemoresistance. Chemoresistance was achieved by a BRCA2 reversion mutation as in C4-2 cells accompanied by a suppression of ssDNA gap induction. See, FIG. 19B. In addition to restored replication fork restraint, these data suggest that chemoresistance is achieved by ssDNA gap filling without replication fork restraint.

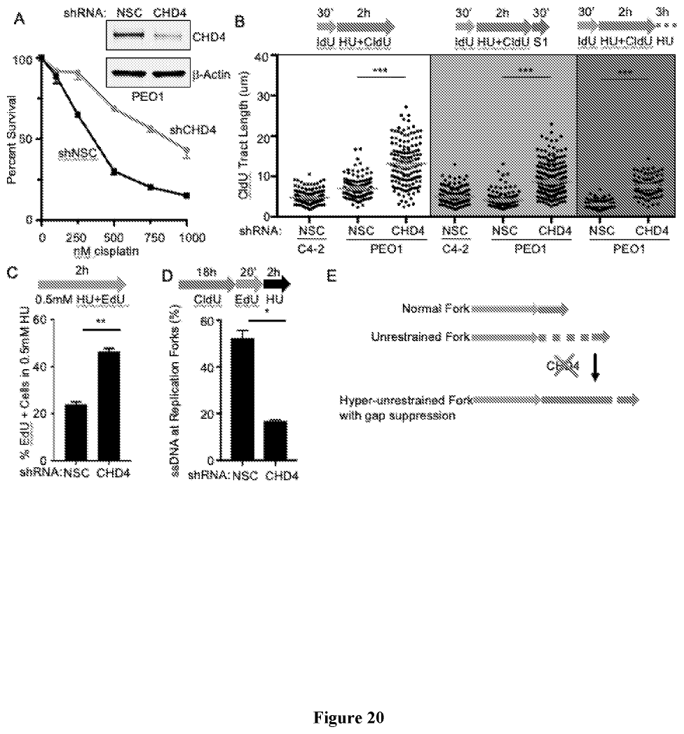

Specifically, BRCA2 deficient cells become chemoresistant by loss of a chromatin remodeler (e.g., CHD49). See, FIG. 20A. CHD4 depletion in PEO1 cells, similar to BRCA2 reversion in C4-2 cells, suppressed S1 nuclease sensitivity and nascent DNA tracks were not degraded after continued exposure to HU. See, FIG. 20B. ssDNA gaps that were adjacent to regions of active replication were reduced when CHD4 was depleted. See, FIG. 20D. However, replication tracks were significantly longer in CHD4-depleted PEO1 cells, compared to PEO1 control or C4-2 cells. See, FIG. 20B. This finding was also reflected in a greater EdU incorporation in CHD4 depleted PEO1 cells as compared to control PEO1 or C4-2 cells in response to HU treatment. See, FIG. 20C. Thus, ssDNA gaps and chemosensitivity were both suppressed by CHD4 depletion, but replication fork restraint was not restored. Instead, replication tracks were longer, indicating replication was further mis-regulated. See FIG. 19E. Previous data found that CHD4 depletion elevated TLS9 suggesting a role for TLS in the dysregulated replication and gap filling (data not shown). Collectively, these data indicate that induction of ssDNA gap formation positively correlate with cancer therapy response and suppression of ssDNA gap formation positively correlates with chemoresistance.

3. The Proteome of BRCA2 Mutant Cells Contains Factors that Mediate and Predict Therapy Response

In one embodiment, the present invention contemplates a method comprising reducing CHD4 activity that results in an activation of TLS, wherein BRCA2 mutant cell ssDNA gap induction is overcome.

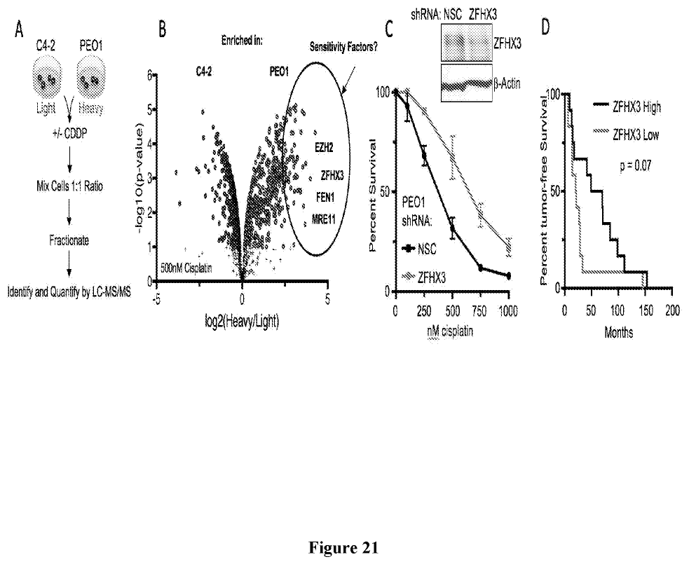

To elucidate protein changes that underlie cancer therapy response in an unbiased manner, high-resolution tandem mass spectrometry was combined with stable isotope labeling with amino acids in cell culture (SILAC)⁴⁸. This technique allows quantitative comparison of the chromatin proteomes between two distinct cell lines. In particular, cisplatin sensitive PEO1 cells were heavily SILAC-labeled and the derived cisplatin resistant clone, C4-2, were lightly SILAC-1abe1ed⁴⁶. See, FIG. 21A. Next, quantitative proteomics revealed the relative abundance of peptides found in chromatin between BRCA2 mutant and proficient cells with and without cisplatin at a dose shown to confer sensitivity in BRCA2 mutant cells⁹. See, FIG. 21B and FIG. 21C. To identify statistically significant changes, all SILAC experiments were performed as biological triplicates. For analysis, average ratios were converted to the log 2 scale for graphing. Finally, p-values were calculated by limma to identify statistically significant changes.

Proteins that were statistically significant and have large-fold changes were prioritized for further analysis. In the two sets of cell comparisons, significant pathways were identified that were up-regulated, represented, and validated along with expected proteins. FIG. 21B. Elevated MRE11, EZH2, a methyltransferase, and FEN1, were found to predict cancer therapy efficacy^{9,49,50}. Moreover, it was found that elevated CHD4-interacting protein ZFHX351, whose depletion enhanced cisplatin resistance in PEO1 cells and of which low mRNA levels predicted poor tumor-free survival in BRCA2 deficient cancer. Seem FIG. 21C and FIG. 21D. These findings indicate that proteomics can identify cancer therapy response genes and suggest biochemical pathways that confer chemoresistance.

4. Loss of Genes Conferring Therapy Resistance and Replication Fork Protection in BRCA2 Deficient Cancer Show ssDNA Gap Suppression

Similar to the CHD4 gene, loss of the EZH2 gene or the FEN1 gene restores FP to BRCA2 deficient cells and has been proposed in the art to mediate cancer therapy resistance in the absence of HR^{9,49,50}. However, the data presented herein showing that CHD4 loss suppresses ssDNA gaps, it was further investigated if the loss of EZH2, FEN1 or ZFHX3 also suppresses ssDNA gaps.

Strikingly, the depletion of ZFHX3 or FEN1, or EZH2 inhibition, increased replication in the presence of HU treatment, as found for CHD4 depletion. See, FIG. 22A. Moreover, inhibition of EZH2 or depletion of ZFHX3 or FEN1 also protected regions of nascent DNA from S1 nuclease degradation in BRCA2 deficient cells, similar to the depletion of CHD4 See, FIG. 22A. Given that CHD4 loss activates TLS, these findings suggest that ssDNA gap suppression via TLS, as opposed to FP via a block to MRE11 degradation, could be a mechanism of chemoresistance common to a variety of cancer therapies. See, FIG. 22B.

5. De Novo Cisplatin Resistant BRCA Deficient Patient Tumors Show Gap Suppression

BRCA patient tumor biopsy samples were tested on a xenograft mouse model to determine if ssDNA gaps could predict chemosensitivity and resistance. A triple-negative breast cancer patient-derived xenograft was selected, PNX0204, that harbored a hemizygous BRCA1 mutation (1105insTC). Following rounds of cisplatin treatment and serial passage in mice, resistant tumors developed. Isogenic sensitive and resistant tumors were then tested for S1 sensitivity, with PEO1 and MDA-MB-436 xenografts serving as controls. See, FIG. 22C. DNA fibers of cisplatin-sensitive PDX cells were degraded by S1 nuclease, but the fibers of cisplatin-resistant isogenic PDX cells were not. See, FIG. 22C. Notably, chemoresistant PDX0204 demonstrated suppressed ssDNA gaps (e.g., gap filling), indicating that loss of ssDNA gaps had occurred in BRCA patient tumors de novo that accurately predicted acquired cisplatin resistance. See, FIG. 22C.

6. HR and FP do not support cisplatin resistance when gaps are present The data disclosed herein present a paradigm-shifting chemotherapeutic strategy that ssDNA gaps cause chemosensitivity and ssDNA gap suppression confers chemoresistance. If true, ssDNA gap suppression should be sufficient to confer chemoresistance even in cells without FP or HR.

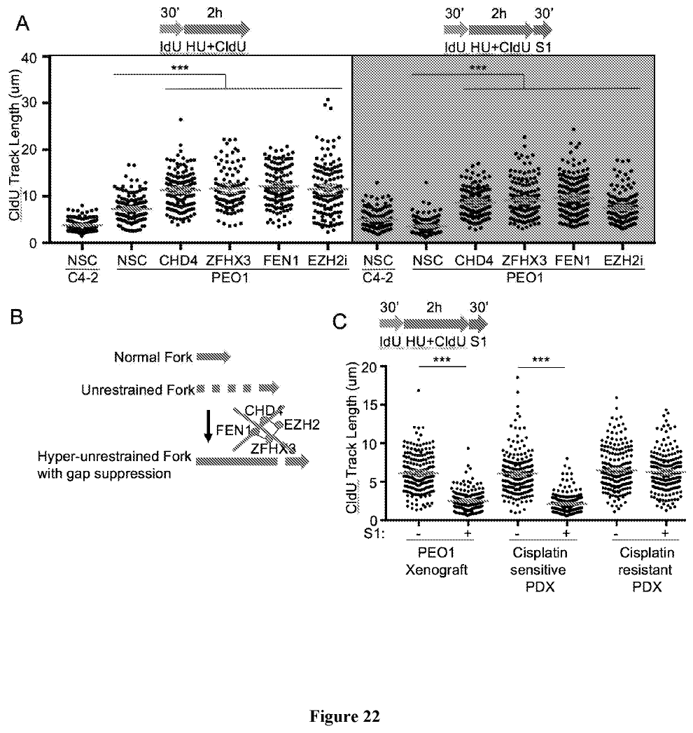

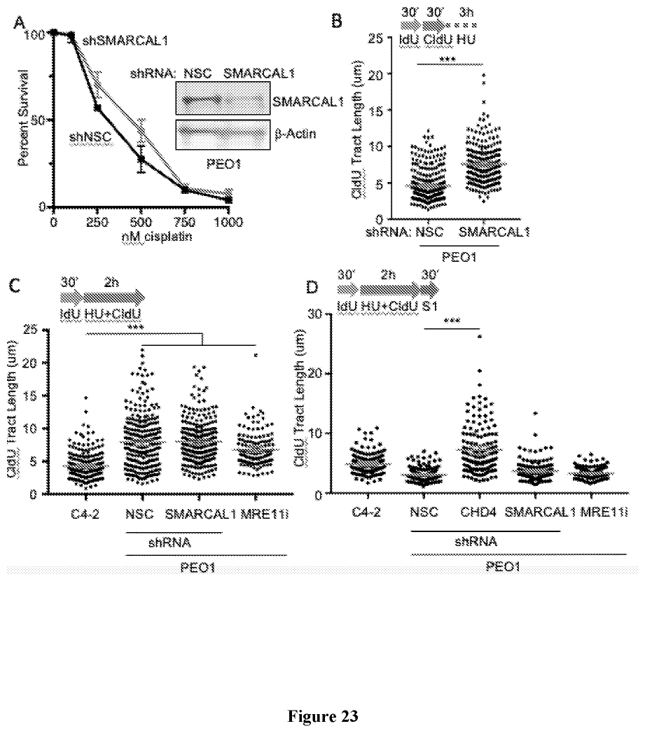

FP was first restored by inhibition of MRE11 or depletion of SMARCAL1 in PEO1 Cells^{39,42,43}. SMARCAL1 depletion did not enhance cisplatin resistance even though FP was restored. See, FIG. 23A and FIG. 23B. However, replication restraint defects and ssDNA gaps were formed with either SMARCAL1 depletion or MRE11 inhibition. See, FIG. 23C and FIG. 23D. These data suggest that FP is insufficient to enhance cisplatin resistance if ssDNA gaps remain. Moreover, ssDNA gaps form independently of MRE11 or SMARCAL1.

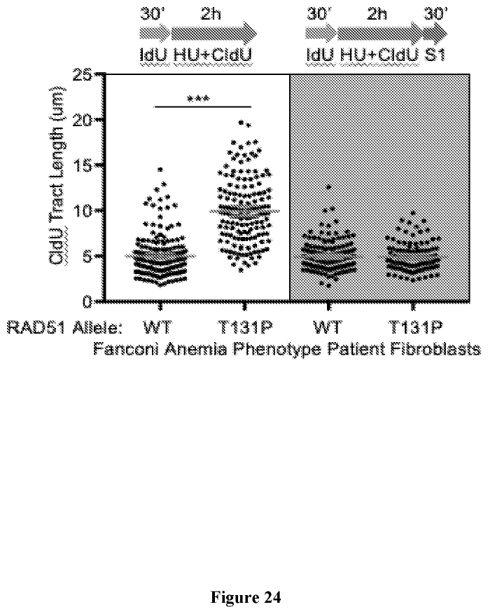

Next, it was determined if similar defects were present in an FA cell line carrying one RAD51^{T131P} mutant allele. These FA cells were also of interest because HR is proficient, but cells are sensitive to cisplatin and PARPi⁵². Moreover, the RAD51^{T131P} mutant is defective for filament formation^{42,53}. Interestingly, in the FA cells, wide-spread ssDNA gap induction was found in the S1 assay as compared to the isogenic RAD51 wild-type (WT) fibroblasts (CRISPR corrected after isolation from the patient)⁵² See, FIG. 24. Together, these results suggest that if ssDNA replication ssDNA gaps are present, cancer cells are sensitive to genotoxic chemotherapies even with an intact HR or FP.

7. Replication Fork Restraint Defects

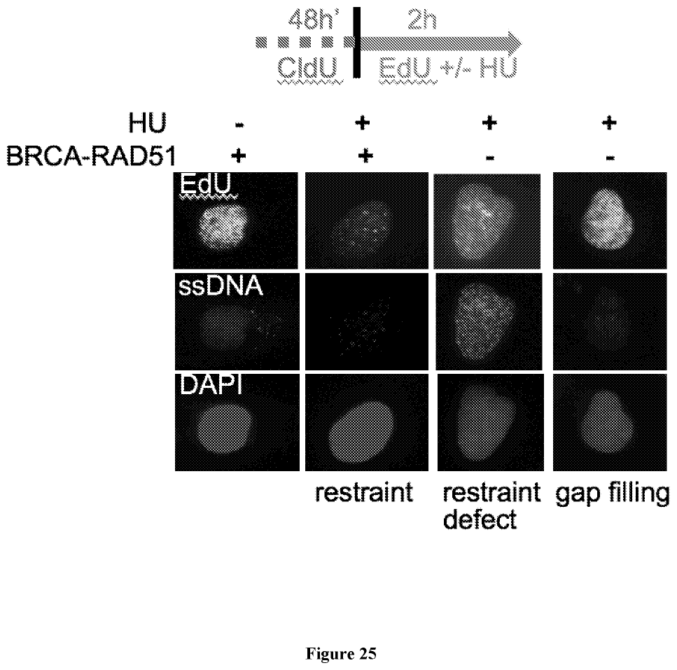

Although it is not necessary to understand the mechanism of an invention, it is believed that unrestrained replication with ssDNA gaps is a feature of other BRCA-RAD51 deficient cells (cancer or non-cancer) and not limited to stress induced by HU treatment. For example, replication stress may be induced by other means. For example, replication stress can be induced with ultraviolet radiation (UV), PARPi and/or aphidicolin treatment, which like HU treatment does not induce lesions. In addition to DNA fiber analysis and S1 nuclease cutting to score the replication stress response (supra), these analyses can be simultaneously scored with EdU incorporation and ssDNA induction by non-denaturing IF to create a combined assay. See, FIG. 25.

While the combined assay does not reveal whether ssDNA gaps are avoided by restored replication restraint or gap filling, it does reveal whether replication occurs with or without ssDNA gaps. For example, in response to HU treatment, BRCA2 proficient C4-2 restrain replication and ssDNA gaps do not form. In contrast, in BRCA2 deficient PEO1 cells, replication continues and ssDNA gaps form, unless CHD4 is depleted and replication proceeds without ssDNA gaps. See, FIG. 25. Given that DNA fibers in BRCA deficient cells treated with DNA interstrand crosslinking (ICLs) agents, such as cisplatin are of poor quality, we can analyze ssDNA gaps by the presently disclosed combined assay to confirm that cisplatin does not arrest replication2, but induces ssDNA gaps⁴⁵ and if these ssDNA gaps are compounded by BRCA deficiency. Collectively, these studies clarify that the BRCA-RAD51 proteins have a role in regulating replication during stress that limits ssDNA gap formation.

8. Molecular Determinants of Replication Fork Restraint

To address the molecular determinants of fork restraint, separation-of-function BRCA2 and RAD51 mutants were created that are defective in at least one function. See, Tables 2 and 3

TABLE 2
RAD51 variants and known functions.
Table 1: RAD51 variants and known functions

| | | Binds | Forms | | Binds | ICL | PARPi | Suppress |
|---|---|---|---|---|---|---|---|---|
| | Domain/motif | DNA | filament | HR | BRC | resistance | resistance | Gaps |
| WT | N/A | +++ | ++ | +++ | +++ | ++++ | ++++ | ++++ |
| T131P | Walker A | ++ | +/− | ++* | ? | − | − | − |
| F86L | NTD/interface | +/− | − | ++++ | ? | ? | ? | ? |
| K133R | Walker A | ++++ | ++++ | − | ? | ? | ? | ? |
| E258A | BRC | + | +/− | +/− | ? | ? | ? | ? |
| | binding | | | | | | | |
| E258K | BRC | ? | ? | ? | ? | ? | ? | ? |
| | binding | | | | | | | |
| E258Q | BRC | ? | ? | ? | ? | ? | ? | ? |
| | binding | | | | | | | |
| Q272L | DNA | + | +/− | +/− | ? | ? | ? | ? |
| | binding | | | | | | | |

RED = somatic tumor variant.
BLUE = germline variant.
GREEN = site-directed mutant.
PURPLE = de novo Fanconi Anemia variant.
*HR in FA cell line
+/− denotes weak or unstable activity.

TABLE 3
BRCA2 variants and known functions.
Table 2: BRCA2 variants and known functions

| | | | | | Binds | Binds | | | |
|---|---|---|---|---|---|---|---|---|---|
| | | Binds | | | RAD51 | RAD51- | ICL | PARPi | Suppress |
| | Domain/motif | DNA | HR | FP | Monomer | Filament | Resistance | Resistance | Gaps |
| WT | N/A | +++ | ++++ | ++++ | ++++ | ++++ | ++++ | ++++ | ++++ |
| [symbol] | PALB2 | ? | − | ? | ++++ | ? | + | ? | ? |
| | interaction | | | | | | | | |
| [symbol] | BRC | ? | ++++ | ? | ++++ | ? | ++++ | ++++ | ? |
| [symbol] | BRC | ++ | ++ | ? | +++ | ? | ++ | ++ | ? |
| [symbol] | C-terminus | +++ | ++++ | − | ++++ | ? | ++++ | ++++ | ? |
| [symbol] | C-terminus | +++ | ++ | − | ++++ | ? | ++ | + | ? |
| BRC4- | BRC repeat | +++ | + | ? | + | + | + | ? | ? |
| DBD | fused-DNA BD | | | | | | | | |
| BRC1-4- | BRC repeats | +++ | ++ | ? | ++ | + | ++ | ? | ? |
| DBD | fused-DNA BD | | | | | | | | |
| BRC5-8- | BRC repeats | +++ | +++ | ? | − | ++++ | +++ | ? | ? |
| DBD | fused-DNA BD | | | | | | | | |

RED = somatic tumor variant.
BLUE = germline variant.
GREEN = site-directed mutant.
PURPLE = fusion protein.
+/− denotes weak or unstable activity.
BD = binding domain [symbol]
indicates data missing or illegible when filed

Several of the mutant constructs are commercially available and have been expressed in human cells^{24,52}. In some embodiments, the present invention contemplates the development of other variants using QuickChange PCR^{14,15}. Alternatively, cancer cells can be genetically engineered using CRISPR-CAS9 technology to knock-in gene mutations of interest into cells proficient or deficient for the BRCA-RAD51 pathway, as routinely done in the art³⁴.

Several K/O and deficient cell lines derived from FA and hereditary breast or ovarian cancer patients that are null or retain residual mutant BRCARAD51 proteins are used herein. These lines provide an opportunity to express a mutant protein and determine which functions are lost or retained. Expressing a gain-of-function mutant (i.e. hyper-filament RAD51 mutant) in a cell deficient for a BRCA protein, provides an opportunity to determine if a mutant protein can rescue loss of a BRCA function. Thus, BRCA1 deficient parental cancer cells lines and therapy resistant derived lines expressing distinct forms of BRCA1 vs WT can be developed and utilized.

Following confirmation of gene mutation by sequencing, or expression by immunoblot analysis, wild-type (WT) vs mutant expressing cells can be evaluated for replication fork restraint and ssDNA gap induction during stress using DNA fiber assay analysis along with a combined assay (supra). To assess the integrity of the pathway, BRCA-RAD51 proteins can be examined for localization to chromatin, foci and complex formation in the presence or absence of replication stress or DNA break induction^{9,19,29}. Collectively, these data disclose domains of the BRCA-RAD51 proteins required for replication fork restraint and ssDNA gap avoidance and prediction of cancer therapy response efficacy.

To ensure that the relationship between replication fork restraint and other BRCA-RAD51 functions can be clearly delineated, mutant BRCA-RAD51 pathway expressing cells can be analyzed for other BRCA-RAD51 functions such as HR and FP. In the event that DNA repair by HR is intact, cells may be able to repair DNA lesions by HR, and cells may be resistant not only to cisplatin, but also to DSB-inducing agents, such as CPT, zeocin or IR. Thus, following treatment with these agents, cell survival experiments can identify these relationships. In particular, some BRCA2 mutants have been complemented with BRCA2 deficient colorectal adenocarcinoma DLD-1 cells and therapy response to cisplatin and PARPi was determined. See, Table 3. Furthermore, following DNA damage, an assessment for a robust RAD51 foci in IF experiments suggests a functional HR pathway^{54,55}. Given an expectation that replication fork restraint correlates with robust RAD51 foci, especially during stress induction, RAD51 filament makers can be tested to make robust foci as compared to filament making mutants when treated with low dose HU. As a control, g-H2AX can be scored by immunoblot to address whether in contrast to break-inducing CPT, low dose HU induced RAD51 foci form independent of DNA damage induction. As before, HR can be directly evaluated using the I-SceI HDR reporter and assess FP in DNA fibers assays^{10,27,34}.

To better understand a biochemical reason for replication restraint defects observed in cells expressing distinct RAD51 variants, these variants can be expressed and purified in vitro^{25,26}. See, Table 2. In comparison to WT RAD51, several parameters can be measured including, but not limited to, filament forming ability on ssDNA or dsDNA, and the ability to localize to and remain at DNA fork or gap structures, using electron microscopy and atomic force microscope imaging, electrophoretic mobility shift assays (EMSA), and quantitative fluorescence quenching assays⁵⁶. The localization of RAD51 proteins bound to fork and gap DNA structures can be determined by nuclease and chemical foot-printing⁵⁷⁻⁵⁹. These DNA substrates can be routinely generated and vary in length and position of DNA forks and ssDNA gaps to determine whether RAD51 variants exhibit unique structure-specific binding^{21,56}.

As shown above, replication fork restraint defects can be detected during a 2 h incubation with low dose HU (0.5 mM) treatment. Replication and ssDNA gaps can further be analyzed in 15-minute increments immediately following HU treatment in order to identify when replication fork restraint defects and ssDNA gaps are first generated. Extending evaluations can determine if fork restraint defects seed subsequent fork degradation and breaks by analyzing these outcomes in 2-12 h increments post-HU treatment. FP may be analyzed as before by DNA fiber and by combined assay (supra). To detect signs of breaks, phosphorylation of RPA32 on S4/S8 can measure a recognized DSB marker and detect breaks by pulse field gel analysis^{60,5}.

Given the possibility that these approaches are not be sensitive enough to reveal minor DSBs, as a control the HU dose can be increased to analyze fork degradation and breaks at a time and dose post-HU treatment known to detect DNA breaks and replication fork degradation^{35,39}. As an additional control, cells may be treated with CPT, at doses shown to induce detectable DSBs⁴⁵. To determine when and if the checkpoint is induced, ATR phosphorylation targets such as g-H2AX, phospho-RPA³², and Chkl may be scored^{12,16,61-63}.

For this analysis, cells can be employed that make ssDNA gaps and are deficient in both HR and FP (e.g., PEO1 cells) and cells that avoid ssDNA gaps and either have both HR and FP, vs deficient in HR but proficient in FP, respectively (e.g., C4-2 or PEO1-CHD4 or SMARCAL1 depleted or inhibited with MRE11)^{39,42,43}. In addition, kinetic analyses can be performed in FP defective, but HR proficient BRCA2 ^{S3192A} expressing BRCA2 deficient VC-8 cells that are not sensitive to chemotherapy and compare this mutant expressed in BRCA2 deficient DLD1 cells39. Moreover, kinetic analyses can also be performed in ssDNA gap making, HR proficient and FP deficient FA cells⁵². In conjunction with a functional domain analysis, this kinetic analysis clarifies the timing of ssDNA gaps and their relationship to replication fork degradation and DNA break induction.

9. Replication Fork Restraint and Replication Stress

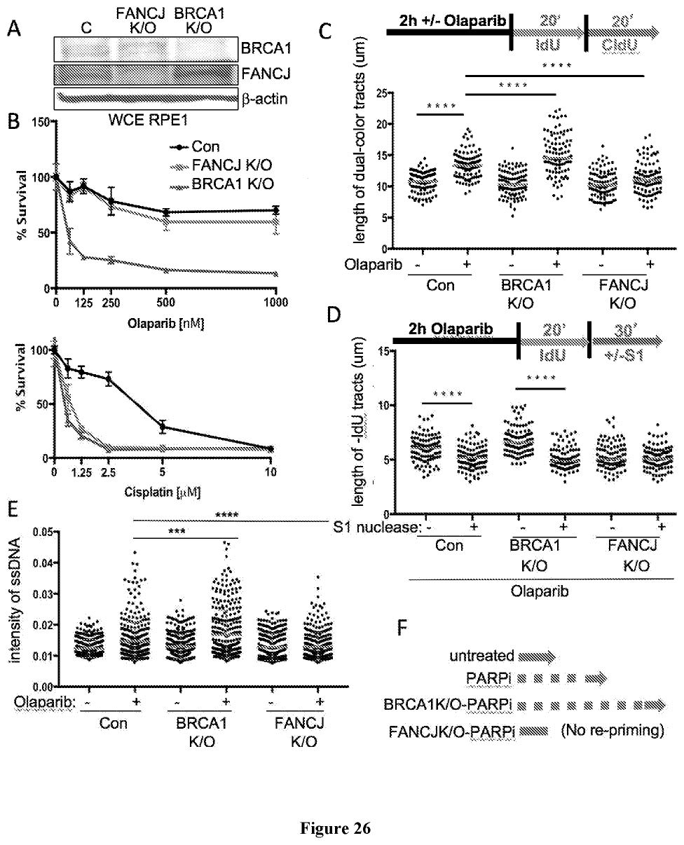

Although it is not necessary to understand the mechanism of an invention, it is believed that BRCA-RAD51 replication fork restraint is functional in response to distinct forms of replication stress and in diverse cell systems. Indeed, BRCA1-K/O RPE cells lengthen with PARPi treatment to a greater extent than controls. See, FIG. 26. While RAD51 filaments at DNA breaks are fundamental for HR, it is believed that filaments are dispensable at replication forks for HR. Consequently, RAD51 filaments may serve as a barrier to the progression of replication forks during stress. For example, the FA cell line with one mutant allele defective for filament formation (e.g., RAD51^{T131P} does not properly slow replication and gaps form even though HR is proficient⁴².

Further analysis of RAD51 tumor variants in cell-based and in vitro assays, can provide molecular and biochemical insight as to whether fork restraint problems derive from loss of RAD51 filament formation, fork or ssDNA binding activity and whether these defects, as opposed to HR or FP defects, contribute to cancer. Further confirmation of this model is demonstrated by showing that gaps in BRCA deficient cells are suppressed by the expression of hyper-filament maker RAD51 mutant K133R even though HR is defective. Replication fork restraint may also require BRCA2 domains involved in chromatin localization and filament formation of RAD51. Indeed, BRCA2 BRC repeats, as well as its C-terminal region, have been reported to be involved in initial RAD51 filament formation⁶⁴. Thus, ssDNA gaps may be prevented by expression of BRC5-8 that promotes RAD51 filaments because of its preference for oligomers over monomers²⁷.

In contrast, ssDNA gaps may be induced by the BRC4 peptide given that it can disrupt RAD51 filaments^{65,66}. It can be predicted that proteomics elucidate other determinants of fork restraint. These factors can be enriched in a BRCA2 proficient cell and reduced in a BRCA2 deficient cell. While an initial gap analysis usually follows HU treatment, the relationship between ssDNA gaps and cancer therapy efficacy, can be determined in the presence of replication stress induced by cisplatin, PARPi et al., which induce ssDNA gaps differentially in distinct cell lines that correlates with sensitivity to the respective agent. For example, RPE cells deficient in either BRCA1 or FANCJ are both sensitive to cisplatin, but only BRCA1 deficient are sensitive to PARPi which also have the most severe PARPi induced gaps. See, FIG. 26C and FIG. 26E. It is further predicted that a kinetic analysis will show that FANCJ null cells do not develop PARPi induced fork degradation or DNA breaks.

C. The Role of ssDNA Gaps in BRCA-Deficient Cancers

Although it is not necessary to understand the mechanism of an invention, it is believed that ssDNA gaps result from re-priming downstream of replication perturbations and can be avoided by restored fork restraint or by gap filling via translesion synthesis (TLS). (supra) By disrupting re-priming vs other replication restart and degradation pathways, gaps are avoided. By activating or inhibiting TLS, gaps are avoided or enhanced, respectively. Lastly, the protein landscape of gap avoidance via fork restraint or TLS that confers chemoresistance can be assessed using quantitative proteomics. In conclusion, it is suggested that loss of the BRCA-RAD51 pathway leads to re-priming induced gaps that are avoided by gap avoidance pathways that rewire the replisome.

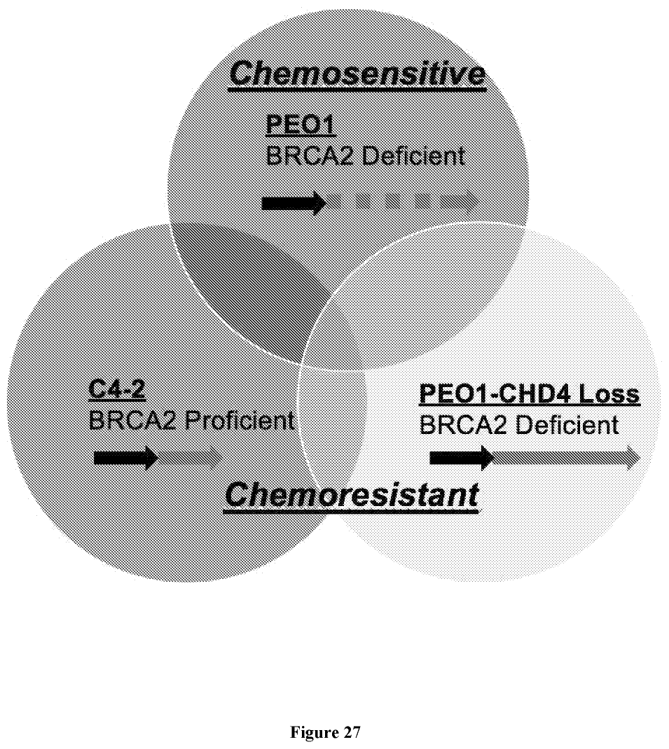

The data presented herein indicate that in human cells the BRCA-RAD51 pathway restrains replication during stress and suppresses ssDNA gap formation. These data also indicate that the accumulation of ssDNA gaps is positively correlated with an efficacious cancer therapy response and that the suppression of ssDNA gaps is positively correlated with a resistance to cancer therapy. Thus, cell models that activate the ssDNA gap making pathway and ensure a therapy response (e.g., PEO1 cells) can be compared with cell models that suppress ssDNA gap pathways (e.g., C4-2 and PEO1-CHD4 deleted cells). Thus, these distinct cell models can comprehensively identify components of chromatin and replisomes by performing iPOND-SILAC (isolation of proteins on nascent DNA—stable isotope labeling of amino acids in cell culture)^{67,34}. See, FIG. 27. Data from a combination of iPOND and proteomics can assess how ssDNA gaps are induced and avoided by fork restraint or TLS activation. These studies can predict how to disrupt replication fork restraint and TLS in cancer to prevent chemoresistance.

The data presented herein provide a description of replication ssDNA gaps in BRCA-RAD51 deficient or mutant cells, but do not define a specific replication intermediate or pathway responsible for these ssDNA gaps. Therefore, whether BRCA-RAD51 directly or indirectly antagonizes replication fork processing events that cause ssDNA gaps such as overactive nucleases or re-priming activity by polymerases is unclear. It is also unclear if ssDNA gap avoidance as with CHD4 depletion, that activates TLS, circumvents issues of fork degradation and collapse that would require BRCA functions in FP and HR. Lastly, identification of replisome machinery for replication fork restraint and TLS can target these pathways for therapeutic strategies to treat BRCA cancer.

1. Re-Priming and ssDNA Gap Induction

Although it is not necessary to understand the mechanism of an invention, it is believed that ssDNA gaps are due to re-priming downstream of transient blocks to DNA polymerization (e.g., DNA sequences, lesions, or other DNA anomalies prone to create secondary structures). Pre-priming may be prevented by a polymerase/primase (e.g., Primpol⁶⁸) or other replication restart pathway (e.g., template switch or transversion^{1,2,69}) and degradation pathways (MRE11, Ctip, Dna2, Mus^{81,70,71}) to determine if ssDNA gap induction is reduced.

Proteins of interest may also be depleted using known inhibitors or siRNA. Upon confirmation of depletion or inhibition in BRCA deficient cells vs controls, replication fork restraint and ssDNA gap formation can be assessed (supra). RAD51 associated polalpha⁴² can also contribute to replication fork restraint and ssDNA gap formation. Polalpha can be depleted or inhibited in cells with or without BRCA2 function and determine whether restraint or gaps are altered⁷². Lastly, given that FANCJ may be involved in PARPi induced gaps, FANCJ may be placed downstream of negative regulation by PARP1 as a re-priming factor. These studies can determine if FANCJ K/O in BRCA1 deficient cells prevents PARPi induced gaps or sensitivity. By comparison, FANCJ K/O cells or double FANCJ and BRCA1 K/O cells can be tested for cisplatin induced gaps and chemosensitivity

2. FEN1, EZH2 or ZHFX3 and TLS Gap Filling

Reduced replication slowing and chromatin remodeling protects nascent DNA in replication forks from degradation; especially in BRCA2 mutant cells in which reversed replication forks are not effectively protected from MRE11 nuclease degradation⁴¹. Moreover, activation of TLS in a BRCA2 mutant cells by CHD4 depletion prevents not only ssDNA gaps (supra), but also fork degradation⁷.

Other factors conferring therapy resistance in BRCA2 mutant cancer may also activate TLS. Likewise, TLS can be activated by other established mechanisms, to assess reductions in fork degradation and cancer therapy sensitivity as found for CHD4 depletion. In particular, over-expression of a TLS scaffolding factors (e.g., REV173, pro-TLS mutant of FANCJ, FANCJ^{S990A})¹⁵, or a depletion of negative regulators of TLS, such as p2174 or the de-ubiquintinase of PCNA monoubiquitination, USP175 can be assessed.

To validate that observed replication fork dynamics or ssDNA gap suppression outcomes are due to TLS, it can be determined if these outcomes are reversed upon depletion or inhibition of the TLS pathway. For these studies, distinct small molecule scaffolds have been identified that disrupt TLS by blocking a protein-protein interaction between the C-terminal domain of REV1 and the Revl interacting region of several other TLS polymerases^{76,77}.

3. The ssDNA Gap Suppression Proteome

The identification of chromatin or replisome factors unique to cells with chemoresistance via ssDNA gap suppression may be addressed by determining whether changes in the chromatin, such as that detected 18 h post cisplatin, occur in the immediate response to stress. Indeed, within 2 h post-HU treatment, accumulated ssDNA gaps may be detected in BRCA2 deficient cells and ssDNA gaps might be suppressed in BRCA2 proficient or deficient cells with CHD4 depletion. See, FIG. 27. Thus, with these cell models, chromatin or iPOND fractions may be purified within 2 h post-HU treatment. These data can be validated by performing immunoblot analysis on iPOND and chromatin samples³⁴. With robust changes, validated proteins can be prioritized based on function in DNA repair, replication, or bypass pathways that are known to confer therapy resistance. To rule out off-target effects of CHD4 depletion, CHD4 depletion with four distinct shRNAs vs NSC in PEO1 cells, generate chromatin lysates and measure candidate protein expression level changes by immunoblot⁹. In addition, proximity ligation assay (PLA)-based approach can be employed to measure an association of proteins to nascent DNA⁷⁸.

4. Enriched Protein Contribution to Chemoresistance

With significant proteins and pathways known to be up-regulated, represented and/or validated in BRCA2 mutant cells with CHD4 depleted, the proteins and pathways can be analyzed their contribution to cisplatin-induced chemoresistance. For this approach, shRNAs are introduced to target the proteins of interest vs control in BRCA2 mutant and proficient cells and protein expression level changes are confirmed by immunoblot. Next, proliferation kinetics can be assessed to determine cell survival following treatment with cisplatin or PARPi. Proteins in which two or more siRNAs reproducibly restore sensitivity can be prioritized for further testing and validation in other cisplatin resistant BRCA1 or BRCA2 deficient cells that have gained cisplatin resistance via HR or FP. With enriched candidates, RNA expression of these protein candidates in BRCA tumors can be evaluated to determine if high expression correlates with poor patient response and/or survival

5. Summary

Although it is not necessary to understand the mechanism of an invention, it is believed that replication ssDNA gaps derive from re-priming-events, rather than by overactive nucleases. For example, the data presented herein shows that ssDNA gaps were unaffected by MRE11 inhibition or depletion of the fork remodeler SMARCAL1, which generates the replication fork structure degraded by MRE11 in BRCA2 deficient cells⁴¹⁻⁴³. Thus, ssDNA gaps arise independently of fork reversal and MRE11, either mediated by a different nuclease or result from re-priming. However, whether these gaps lengthen or shrink when cells are treated with MRE11i or depleted of Primpol, respectively, should be considered.

Conceivably, RAD51 chromatin loading serves to stabilize and promote the progression of polymerases via its interaction with polalpha⁴². Alternatively, RAD51 could sequester polalpha so that other polymerases are favored. In this case, ssDNA gaps in BRCA-RAD51 deficient cells could derive from unregulated polalpha. Thus, whether loss of polalpha, as opposed to Primpol, reduces gaps in BRCA1 deficient cells should be considered. Proteins and pathways identified to be uniquely upregulated and/or expressed in ssDNA gap cell models provide information relevant to ssDNA gap formation, but also provide information that predicts therapy efficacy. By performing iPOND or chromatin analysis at time points prior to, and after, ssDNA gap formation the protein landscape of stress signaling and subsequent fork degradation or break induction can be defined. Likewise, iPOND-immunoblot experiments following cisplatin or PARPi can be performed to determine if the proteome of gap formation/therapy response or gap avoidance/therapy resistance is similar irrespective of the source of stress.

D. ssDNA Gap Suppression and Chemoresistance

In one embodiment, the present invention contemplates that BRCA deficient cells are sensitive to cancer therapies because of the accumulation of ssDNA gaps. In one embodiment, chemoresistance develops when ssDNA gaps are suppressed by restored fork restraint or gap filling. Although it is not necessary to understand the mechanism of an invention, it is believed that extensive gap induction is a mechanism by which BRCA deficient cells are sensitized to cancer therapy and, conversely, ssDNA gap suppression mediates chemoresistance. To assess the contribution gap suppression by restored fork restraint or gap filling to therapy resistance, ssDNA gap formation was measured in distinct isogenic models of restored FP and/or HR as well as de novo models of chemoresistance using human cell lines and patient samples. In addition, a determination of whether there is a correlation between genes (whose loss or enrichment) suppresses ssDNA gaps with poor patient response to cancer therapy using available cancer data sets. In one embodiment, the present invention contemplates that ssDNA gaps predicts cancer therapy response and chemoresistance outcomes in cancers.

PARPi(s) are being used in the clinic and in combination with cisplatin to treat BRCA and HR-defective cancer ⁴. The conventional mechanism{s} assumed for cancer cell death is premised upon the induction of persistent DNA double strand breaks and replication fork degradation. Moreover, mechanisms described in the art regarding chemoresistance invokes restored HR and FP. However, this belief does not address recent reports in which it was shown that these drugs do not initially pause replication or induce breaks³. Thus, some embodiments of the present invention re-evaluates mechanism(s) of cancer cell death induced by chemotherapeutics.

1. PARPi Induced Gaps are Enhanced in BRCA1 Deficient Cells and Avoided in FANCJ Deficient Cells.

To address a mechanism of action of a PARPi, it was determined if, similar to BRCA1 deficient cells, FANCJ deficient cells were sensitive to the PARPi, olaparib⁸⁷. While BRCA1 K/O cells were sensitive to olaparib, FANCJ K/O cells were not sensitize to olapraib although both K/O lines were sensitive to cisplatin. See, FIG. 26A. It was also confirmed that PARPi lengthens replication tracts after 2 h with 10 μM olaparib. See, FIG. 26C. Further indicating that BRCA deficiency causes replication restraint defects in response to stress, following PARPi treatment, the BRCA1 K/O cells displayed longer replication tracts than control. See, FIG. 26C.

Consistent with PARPi inducing gaps, the longer DNA replication tracts following PARPi treatment were significantly shortened when cells were treated with the S1 nuclease prior to DNA spreading. See, FIG. 26D. Moreover, non-denaturing IF revealed ssDNA was induced by PARPi more dramatically in BRCA1 K/O cells compared to control. See, FIG. 26E. In contrast, replication fork tracts in FANCJ K/O RPE cells did not lengthen or have a change in length when cells were treated with S1 nuclease. See, FIG. 26D. Moreover, PARPi induced ssDNA genomic gaps were lower in FANCJ K/O as compared to control or BRCA1 K/O RPE cells. See, FIG. 26E. These findings indicate that PARPi causes replication lengthening and ssDNA gaps that are exacerbated in BRCA K/O cells and avoided in FANCJ K/O cells, perhaps because FANCJ is required for re-priming in response to PARP loss. See, FIG. 26F. Although it is not necessary to understand the mechanism of an invention, it is believed that FANCJ drives re-priming and FANCJ deletion disrupts PARPi sensitivity in BRCA deficient cells.

Since FANCJ deficiency is believed to disrupt both HR and FP^{10,34}, the above data argue that defects in HR or FP are not a primary mediator of cancer therapy sensitivity to PARPi. Furthermore, replication ssDNA gaps, not fork degradation or breaks could play a role in any mechanism of action of first-line genotoxic chemotherapies that sensitize BRCA deficient cells. Furthermore, ssDNA gap suppression, not restored HR or FP, could be a mechanism of chemoresistance for all cancer therapies. Such a cancer therapy strategy is supported by showing that HR and FP can be fully uncoupled from PARPi resistance in diverse genetic and de novo models of resistance.

2. PARPi Chemoresistance Via CHD4 Loss and TLS and ssDNA Gap Suppression

Similar to loss of CHD4, loss of PTIP, EZH2, or FEN1 predicts poor patient response in BRCA2 cancer and suppresses fork degradation in DNA fiber assays following HU treatment^{9,49,50}. It can be determined whether loss of either of these genes will also suppress ssDNA gaps induced by PARPi. First, depletion can be confirmed by immunoblot and that similar to loss of CHD4, loss of EZH2, PTIP or FEN1 in BRCA2 deficient cells confers chemoresistance and FP^{9,49,50}. Next, DNA fiber with S1 nuclease cutting assay can be performed and a combined IF assay assesses global gap formation and replication proficiency. Analysis of DNA fibers also allows a determination of whether PARPi resistance is established by ssDNA gap suppression that occurs via gap filling as found with CHD4 depletion or via restored fork restraint as found with FANCJ deficiency. In addition, TLS can be activated by other means to determine if PARPi-induced gaps or sensitivity are suppressed.

3. HR and/or FP Restoration of PARPi Resistance in ssDNA Gap Presence

In one embodiment, the present invention contemplates that that PARPi induced gaps cause chemosensitivity and gap suppression confers chemoresistance independent of FP or HR, PARPi induced sensitivity, replication fork tract lengths, tract length S1 nuclease sensitivity, and global ssDNA and EdU incorporation can be determined in cells with FP, but no HR (PEO1 cells with MRE11 inhibition or depletion of SMARCAL1 or in cells with HR, but no FP (FA cells with the RAD51 T131P mutant. These data may show that PARPi induced gaps are more pronounced in sensitive lines even when FP and HR are intact.

4. ssDNA Gap Suppression in Genetic Models of Restored HR and PARPi Resistance

Although it is not necessary to understand the mechanism of an invention, it is believed that suppression of PARPi induced ssDNA gaps also occurs in distinct models of chemoresistance that have been attributed to restored HR. One well described mechanism of PARPi resistance in BRCA1 deficient cancer cells is by loss of the DNA repair protein, 53BP1, that limits HR by blocking DNA end resection⁸⁸. The 53BP1-interacting proteins RIF1, shieldin, PTIP, REV7 and polalpha have DNA end blocking activity that prevent the resection of DSBs in BRCA1-deficient cells^{72,89}. Consequently, when this complex is intact, it is proposed that BRCA1 mutant cells are highly sensitive to PARPi; conversely, loss of complex components results in PARPi resistance. However, if PARPi do not make breaks, loss of 53BP1 could promote PARPi resistance not by restoring HR, but rather by suppressing gaps.

PARPi resistance resulting from gap suppression can be determined in the presence or absence of PARPi treatment in RPE cells with either BRCA1 K/O, or double K/O of BRCA1 and 53BP1, or double K/O of BRCA1 and shieldlin⁸⁹. In addition, depletion of 53BP1, and associated partners (shieldin, polalpha, Ptip) in BRCA1 deficient breast cancer cell lines can determine if PARPi induced resistance and gap suppression are elevated compared to controls. Together, these findings may suggest that PARPi resistance in BRCA1 deficient cells as achieved by 53BP1 loss is also linked to ssDNA gap suppression. Again, by examining DNA fiber lengths, it can be determined if resistance is achieved by restored fork restraint, or by gap filling. BRCA1 hypomorphs are expressed in tissues and cancers, however their functions are not fully appreciated.

The data presented herein indicate that hypomorphs confer therapy resistance. In particular, removal of deleterious germline BRCA1 mutations through alternative mRNA splicing gives rise to isoforms that retain residual activity and contribute to therapeutic resistance. In particular, the BRCA1-Δ11q isoform derives from the alternative choice of splice donor site within BRCA1 exon 11, resulting in the loss of the majority of exon 11 nucleotides (c.788-4096/aa. 263-1365). Relative to full-length BRCA1, the splice form, BRCA1-Δ11q is partially functional for HR. Moreover, the splice isoform, BRCA1-Δ11q confers cisplatin and PARPi resistance¹⁹. These findings provide an explanation for why BRCA1 exon 11 mutation carriers have significantly worse overall survival compared to patients with mutations outside of this exon.

In addition, another model was established using both cisplatin and PARPi resistance working with the SUM1315MO02 cell line that was derived from a skin metastasis of a female patient with invasive ductal carcinoma²⁰. These cells harbor a hemizygous BRCA1185delAG mutation. The SUM1315MO2 cells are initially sensitive to PARPi and cisplatin but acquired resistance in ˜1 to 2 months when cultured in the presence of increasing concentrations of either the PARPi or cisplatin. Resistant clones do not have secondary reversion mutations, but rather, gain increased expression of a “really interesting gene” (RING) domain—deficient BRCA1 protein (Rdd-BRCA1). For example, Rdd-BRCA1 protein expression was detected in recurrent carcinomas from patients who carried germline BRCA1185delAG mutations suggesting this is a clinically relevant mechanism of resistance in BRCA1 cancer²⁰. To address whether therapy resistance in these models stem from gap suppression, a BRCA1 deficient breast cancer cell line can be employed that expresses a splice form (e.g., L56BRC1, SUM149PT, UWB1.289) or the sensitive vs resistant derived SUM1315MO2 cells that gain the Rdd-BRCA1 along with established PDX models derived from BRCA1 exon 11 or BRCA1185delAG mutant cancers. Manipulation of BRCA1 isoform expression using CRISPR/Cas9 is performed to examine the impact of hypomorph expression on ssDNA gap formation in response to HU, cisplatin or PARPi.

5. PARPi Resistance in BRCA2 Deficient Patient Tumors and ssDNA Gap Suppression

Although it is not necessary to understand the mechanism of an invention, it is believed that ssDNA gap avoidance mechanisms are detected in chemoresistant cell lines and patient samples that develop chemoresistance de novo.

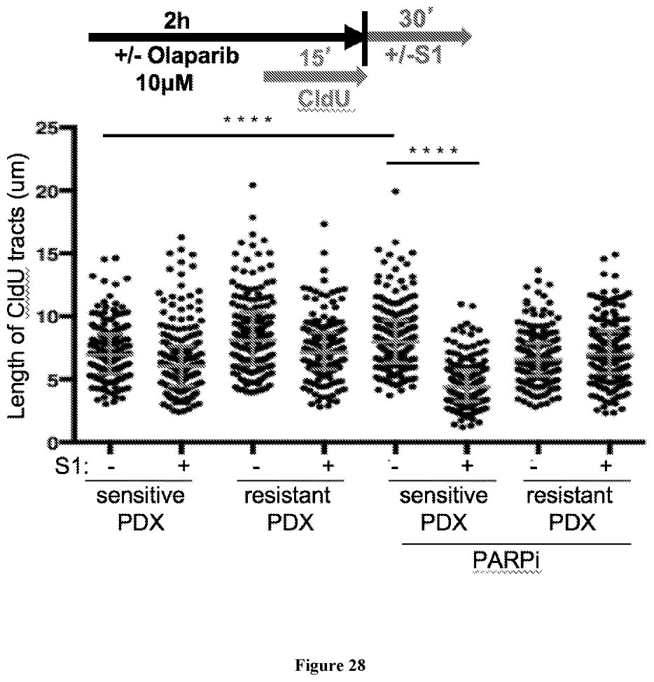

Several initially PARPi sensitive cell lines and patient derived xenograft (PDX) samples have been established that developed PARPi resistance under drug exposure when propagated in tissue culture or upon serial passage in mice^{17,20}. ssDNA gap suppression can be observed to occur by a combined assay and analyzed DNA fibers to address if ssDNA gap filling is achieved by TLS or restored restraint. In a preliminary experiment, it was found that as compared to the therapy sensitive PDX, gaps as measured in the S1 nuclease assay were suppressed in the resistant PDX following PARPi treatment. See, FIG. 28. (FIG. 11). It was also noted that replication tracts lengthened in the sensitive PDX, whereas the resistant PDX did not lengthen upon PARPi treatment, suggesting that ssDNA gaps were avoided by a block to lengthening or re-priming similar to loss of FANCJ (supra).

6. ssDNA Gap Suppression Predicts Cancer Therapy Efficacy

In one embodiment, the present invention contemplates a method comprising treating a patient sample with a chemotherapeutic and predicting chemoresistance by detecting ssDNA gap suppression in said treated patent sample. Although it is not necessary to understand the mechanism of an invention, it is believed that continued use of publicly available data sets can test the predictive power of genes (whose loss or gain of expression) to suppress ssDNA gaps in tissue culture and/or PDX models. The proteomics dataset presented herein has promise for identifying what is unique to a PEO1-(BRCA2 deficient cancer) and is part of the “gap making machinery”. Indeed, this data set has good predictive power because several of the genes (EZH3, ZFHX3, FEN1) are required for ssDNA gap making and their loss predicts poor patient response (supra). In addition, identifying what is unique to a PEO1-CHD4 depleted (TLS activated) and C4-2 (BRCA2 proficient cells) can supplement the ongoing “gap suppressing machinery” model development.

7. Summary

Although it is not necessary to understand the mechanism of an invention, it is believed that ssDNA gap suppression occurs by both restored replication restraint or by ssDNA gap filling via TLS. Suppression of ssDNA gap formation predicts PARPi resistance in patient xenografts. Schematic and quantification of CldU track lengths in S1 cutting assay in BRCA1 deficient patient derived xenografts. also suggest that in either model some of the elevated proteins found trend with high levels of their mRNAs predicting poor patient response in BRCA2 deficient cancer. As previously found, low CHD4 mRNA predicts poor patient response in BRCA2 patients'. If lower-fidelity TLS polymerases⁹⁰ participate in ssDNA gap filling as the present invention predicts, this process could contribute to developing a base substitution signature typical of BRCA2-defective cancer cells⁹¹.

The data presented herein suggest that drugs or genetic perturbations leading to synthetic lethal interactions in BRCA deficient cancer cells such as WEE1 inhibitors or poltheta loss also may cause a toxic accumulation of ssDNA gaps, or that TLS inhibition restores cisplatin or PARPi sensitivity. Conceivably, cancer cells with high levels of intrinsic stress uniquely employ TLS to maintain replication whereas non-cancer cells rely on the normal replicative polymerases. If true, TLSi therapy could be selective to cancer cells and spare non-cancer cells.

The persistence of unrepaired DNA breaks and degraded replication forks are thought to underlie the sensitivity of tumors deficient in the hereditary breast cancer genes to cisplatin or PARPi. Challenging this assumption, recent findings indicate that these chemotherapies do not initially cause DNA breaks or stall forks. The data presented herein suggest an alternative paradigm changing model. Specifically, it is revealed that ssDNA gaps derive from a failure of replication restraint during stress and that these cells are hypersensitive to chemotherapy. Furthermore, resistance to chemotherapy develops when ssDNA gaps are avoided when either fork restraint is restored, or ssDNA gaps are filled. Thus, ssDNA gaps may underlie the hypersensitivity of BRCA deficient cancer.

It can be determined if ssDNA gaps accurately predicts chemoresponse in cell culture, using the TCGA patient database, and patient xenografts. It can also be specifically determined if an ssDNA gap model is predictive even in cases in which the current dogma, namely the models of HR and FP, break down. For example, series of separation-of-function mutants in BRCA-RAD51 genes have been developed that are proficient or deficient for distinct functions that enable as assessment of whether HR or FP can be uncoupled from cancer therapy response. Collectively, embodiments of the present invention define ssDNA gaps as an underlying defect that may control global cancer phenotypes. One example, “BRCAness,” provides much needed new insight towards understanding the mechanism of action of chemotherapies and how chemoresistance develops.

E. Gap Inducing Therapies are Also Evaded by TLS

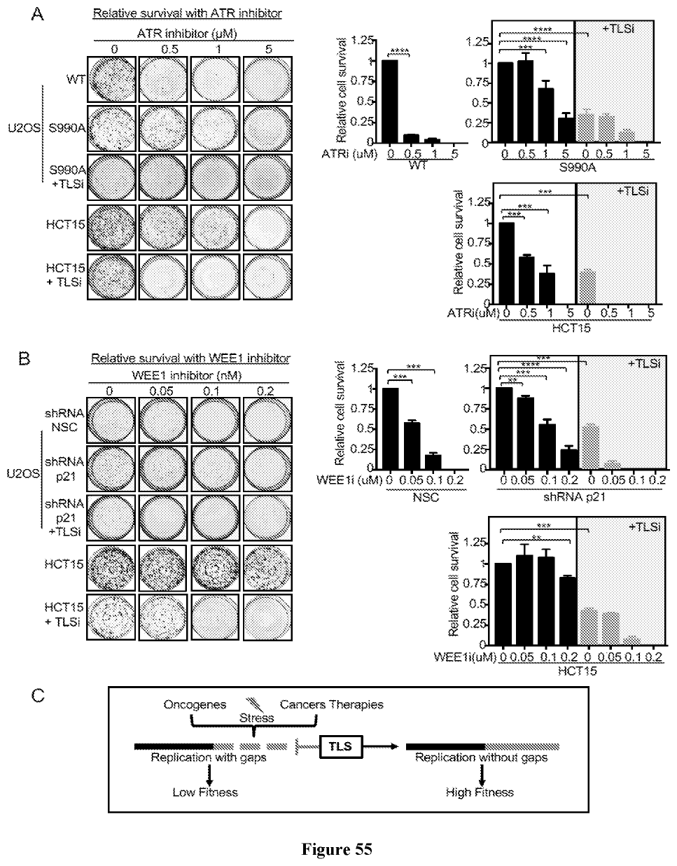

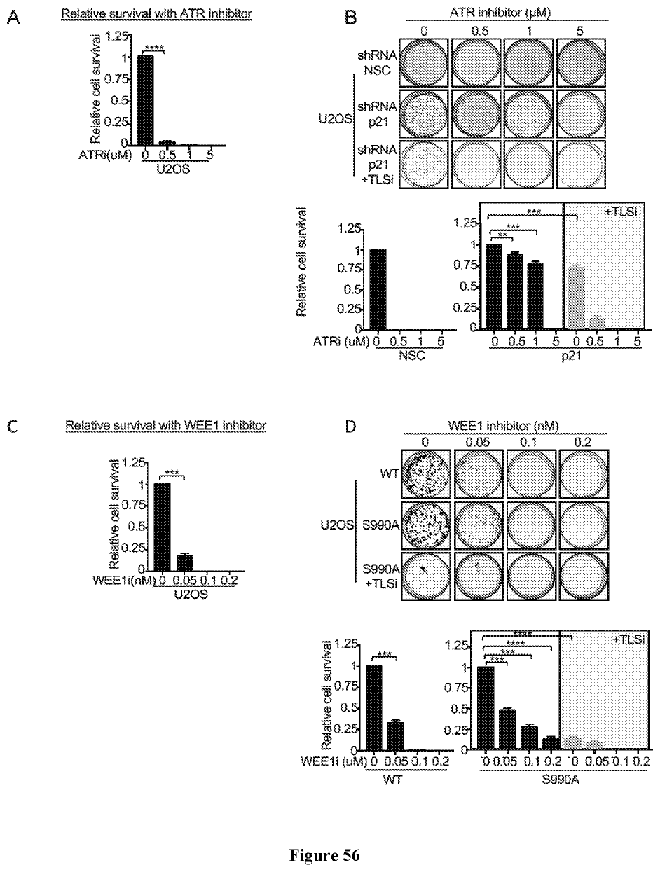

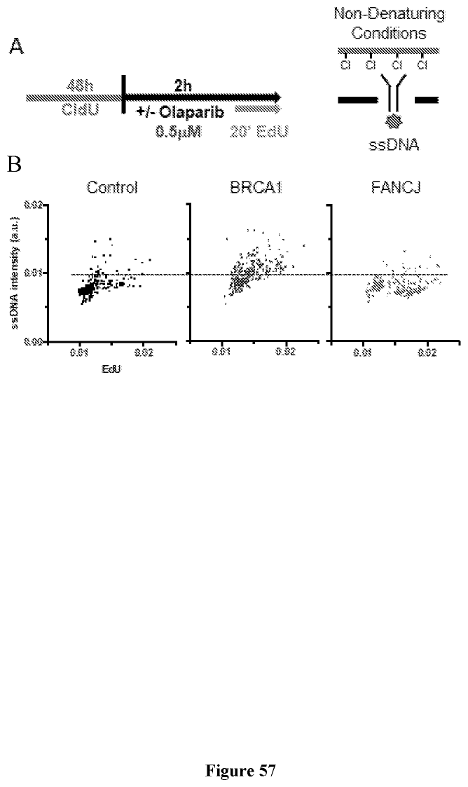

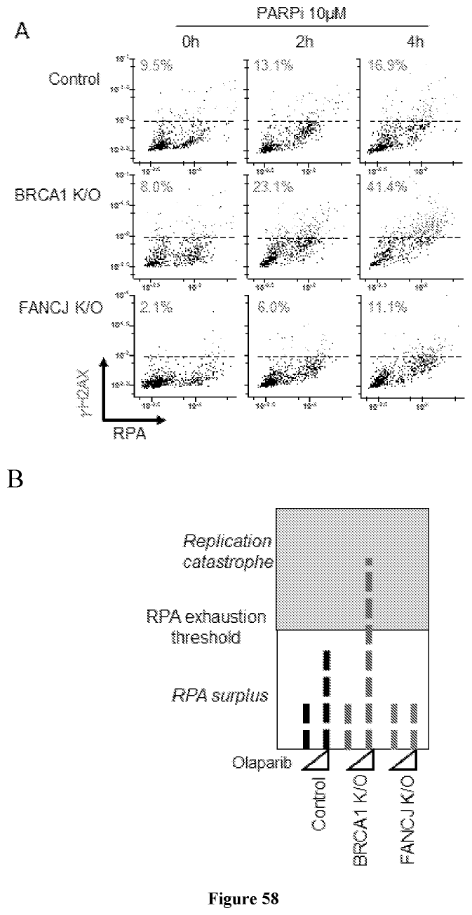

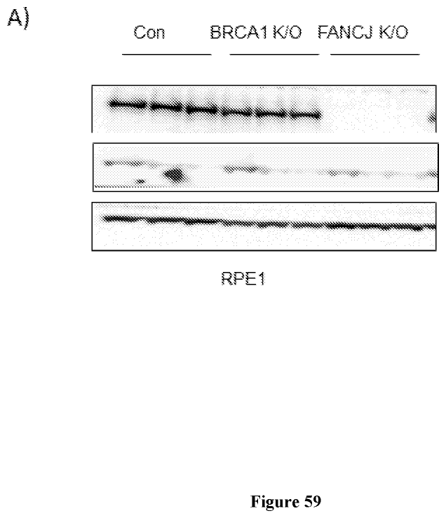

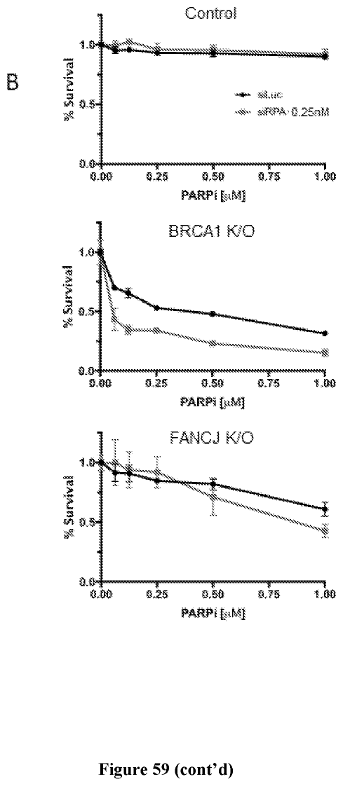

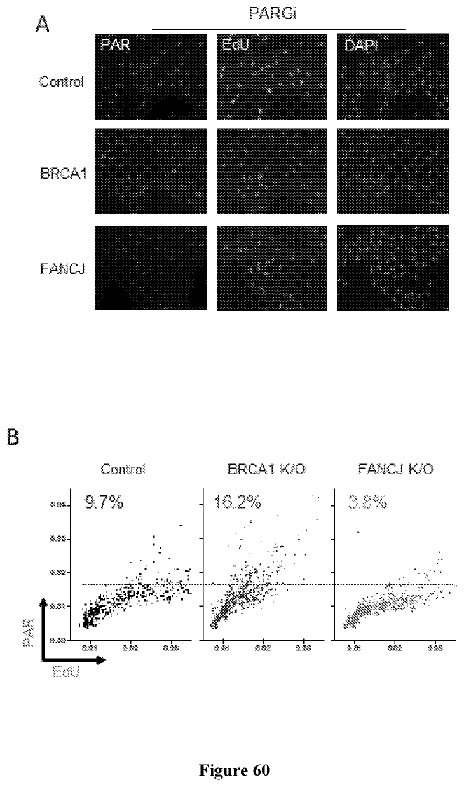

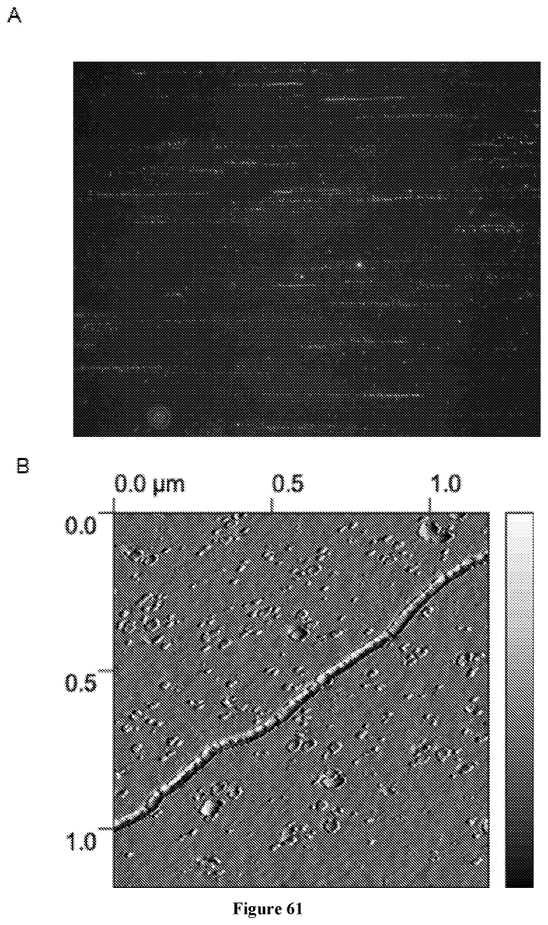

Currently, there is a major clinical effort to combat cancer by induction of replication stress through inhibition of ATR or the mitotic checkpoint kinase, Wee1 (Bukhari et al., 2019). Given that these drugs induce replication gaps, it was considered that if gaps were the sensitizing lesion, TLS could also interfere with their effectiveness. See, FIG. 29A; (Couch et al., 2013; Forment and O'Connor, 2018; Yang et al., 2017), Indeed, compared to the non-TLS dependent cells, the pro-TLS U2OS cells had greater clonogenic survival following ATRi or Wee1i, inhibitor MK1775. Similarly, the pro-TLS cancer cell line, HCT15, also showed resistance to both the ATRi and Wee1i. See, FIG. 55A, 5B and FIG. 56A-D; (Bukhari et al., 2019). However, when co-incubated with the TLSi, the pro-TLS U2OS or HCT15 cell lines were re-sensitized suggesting a more potent therapeutic response when TLSi is used in combination with ATRi or Wee1i

Collectively, these findings demonstrate that TLS overcomes replication stress from oncogene expression that explains the prevalence of cancer cells rewired to depend on TLS for replication and fitness. This TLS rewiring mitigates the effectiveness of drugs such that inhibit ATR and Weel, that induce gaps, suggesting the greater clinical potential of targeting TLS as a cancer therapy. FIG. 55C.

The replication stress response is a robust protective barrier that arrests or eradicates cells that are undergoing replication stress. Thus, cancers that overcome this barrier are expected to suppress the stress response by some mechanism. It is predicted that rewired replication that favors TLS represents an adaptation to blunt oncogene induced replication stress that otherwise rapidly induces ssDNA gaps and limits fitness. In support of this model, distinct cancer cells rely on TLS for fitness and to replicate during extrinsic or intrinsic stress. Mechanistically, it has been uncovered that TLS curtails the slowing and remodeling of replication forks and the global replication arrest response while also suppressing ssDNA gaps that are toxic to cells (Chen et al., 1997; Forment and O'Connor, 2018; Gagna et al., 2000; Huang et al., 1996; Naruse et al., 1994; Nur et al., 2003; Peitsch et al., 1993; Tidd et al., 2000). Collectively, the present findings highlight the importance of replication gaps as a cancer vulnerability that could be leveraged by TLS inhibition alone or in conjunction with gap inducing therapies in a wide range of cancers.

### REFERENCES: SECTIONS I-V

1 Zellweger, R. et al. Rad51-mediated replication fork reversal is a global response to genotoxic treatments in human cells. J Cell Biol 208, 563-579, doi:10.1083/jcb.201406099 (2015).
2 Neelsen, K. J. & Lopes, M. Replication fork reversal in eukaryotes: from dead end to dynamic response. Nat Rev Mol Cell Biol 16, 207-220, doi:10.1038/nrm3935 (2015).
3 Mutreja, K. et al. ATR-Mediated Global Fork Slowing and Reversal Assist Fork Traverse and Prevent Chromosomal Breakage at DNA Interstrand Cross-Links. Cell Rep 24, 2629-2642 e2625, doi:10.1016/j.celrep.2018.08.019 (2018).
4 Bartkova, J. et al. Oncogene-induced senescence is part of the tumorigenesis barrier imposed by DNA damage checkpoints. Nature 444, 633-637, doi:10.1038/nature05268 (2006).
5 Macheret, M. & Halazonetis, T. D. DNA replication stress as a hallmark of cancer. Annu Rev Pathol 10, 425-448, doi:10.1146/annurev-pathol-012414-040424 (2015).
6 Roy, S. et al. p53 orchestrates DNA replication restart homeostasis by suppressing mutagenic RAD52 and POLtheta pathways. Elife 7, doi:10.7554/eLife.31723 (2018).
7 Couch, F. B. et al. ATR phosphorylates SMARCAL1 to prevent replication fork collapse. Genes Dev 27, 1610-1623, doi:10.1101/gad.214080.113 (2013).
8 Qiu, Z., Oleinick, N. L. & Zhang, J. ATR/CHK1 inhibitors and cancer therapy. Radiother Oncol 126, 450-464, doi:10.1016/j.radonc.2017.09.043 (2018).
9 Pilie, P. G., Tang, C., Mills, G. B. & Yap, T. A. State-of-the-art strategies for targeting the DNA damage response in cancer. Nat Rev Clin Oncol 16, 81-104, doi:10.1038/s41571-018-0114-z (2019).
10 Kannouche, P. L. & Lehmann, A. R. Ubiquitination of PCNA and the polymerase switch in human cells. Cell Cycle 3, 1011-1013 (2004).
11 Barnes, R. & Eckert, K. Maintenance of Genome Integrity: How Mammalian Cells Orchestrate Genome Duplication by Coordinating Replicative and Specialized DNA Polymerases. Genes (Basel) 8, doi:10.3390/genes8010019 (2017).
12 Vujanovic, M. et al. Replication Fork Slowing and Reversal upon DNA Damage Require PCNA Polyubiquitination and ZRANB3 DNA Translocase Activity. Mol Cell 67, 882-890 e885, doi:10.1016/j.molce1.2017.08.010 (2017).
13 Xie, J. et al. FANCJ/BACH1 acetylation at lysine 1249 regulates the DNA damage response. PLoS genetics 8, e1002786, doi:10.1371/journal.pgen.1002786 (2012).
14 Xie, J. et al. Targeting the FANCJ-BRCA1 interaction promotes a switch from recombination to poleta-dependent bypass. Oncogene 29, 2499-2508, doi:10.1038/onc.2010.18 (2010).
15 Quinet, A., Carvajal-Maldonado, D., Lemacon, D. & Vindigni, A. DNA Fiber Analysis: Mind the Gap! Methods Enzymol 591, 55-82, doi:10.1016/bs.mie.2017.03.019 (2017).
16 Korzhnev, D. M. & Hadden, M. K. Targeting the Translesion Synthesis Pathway for the Development of Anti-Cancer Chemotherapeutics. J Med Chem 59, 9321-9336, doi:10.1021/acs.jmedchem.6b00596 (2016).
17 Sail, V. et al. Identification of Small Molecule Translesion Synthesis Inhibitors That Target the Rev1-CT/RIR Protein-Protein Interaction. ACS Chem Biol 12, 1903-1912, doi:10.1021/acschembio.6b01144 (2017).
18 Yang, Y. et al. Diverse roles of RAD18 and Y-family DNA polymerases in tumorigenesis. Cell Cycle 17, 833-843, doi:10.1080/15384101.2018.1456296 (2018).
19 Garcia-Exposito, L. et al. Proteomic Profiling Reveals a Specific Role for Translesion DNA Polymerase eta in the Alternative Lengthening of Telomeres. Cell Rep 17, 1858-1871, doi:10.1016/j.celrep.2016.10.048 (2016).
20 Wang, B. et al. Abraxas and RAP80 form a BRCA1 protein complex required for the DNA damage response. Science 316, 1194-1198, doi:316/5828/1194 [pii]10.1126/science.1139476 (2007).
21 Greenberg, R. A. et al. Multifactorial contributions to an acute DNA damage response by BRCA1/BARD1-containing complexes. Genes Dev 20, 34-46 (2006).
22 Kumaraswamy, E. & Shiekhattar, R. Activation of BRCA1/BRCA2-associated helicase BACH1 is required for timely progression through S phase. Mol Cell Biol 27, 6733-6741, doi:MCB.00961-07 [pii]10.1128/MCB.00961-07 (2007).
23 Wu, Y. & Brosh, R. M., Jr. FANCJ helicase operates in the Fanconi Anemia DNA repair pathway and the response to replicational stress. Curr Mol Med 9, 470-482 (2009).
24 Gong, Z., Kim, J. E., Leung, C. C., Glover, J. N. & Chen, J. BACH1/FANCJ acts with TopBP1 and participates early in DNA replication checkpoint control. Molecular cell 37, 438-446, doi:10.1016/j.molce1.2010.01.002 (2010).
25 Sarkies, P. et al. FANCJ coordinates two pathways that maintain epigenetic stability at G-quadruplex DNA. Nucleic Acids Res 40, 1485-1498, doi:10.1093/nar/gkr868 (2012).
26 Schwab, R. A., Nieminuszczy, J., Shin-Ya, K. & Niedzwiedz, W. FANCJ couples replication past natural fork barriers with maintenance of chromatin structure. The Journal of cell biology 201, 33-48 (2013).
27 Raghunandan, M., Chaudhury, I., Kelich, S. L., Hanenberg, H. & Sobeck, A. FANCD2, FANCJ and BRCA2 cooperate to promote replication fork recovery independently of the Fanconi Anemia core complex. Cell Cycle 14, 342-353, doi:10.4161/15384101.2014.987614 (2015).
28 Matsuzaki, K., Borel, V., Adelman, C. A., Schindler, D. & Boulton, S. J. FANCJ suppresses microsatellite instability and lymphomagenesis independent of the Fanconi anemia pathway. Genes Dev 29, 2532-2546, doi:10.1101/gad.272740.115 (2015).
29 Cantor, S. et al. The BRCA1-associated protein BACH1 is a DNA helicase targeted by clinically relevant inactivating mutations. Proc Natl Acad Sci U S A 101, 2357-2362 (2004).
30 Cantor, S. B. et al. BACH1, a novel helicase-like protein, interacts directly with BRCA1 and contributes to its DNA repair function. Cell 105, 149-160 (2001).
31 Chaudhuri, A. R. et al. Replication fork stability confers chemoresistance in BRCA-deficient cells. Nature 535, 382-387, doi:10.1038/nature18325 (2016).
32 Guillemette, S. et al. FANCJ localization by mismatch repair is vital to maintain genomic integrity after UV irradiation. Cancer research 74, 932-944 (2014).
33 Guillemette, S. et al. Resistance to therapy in BRCA2 mutant cells due to loss of the nucleosome remodeling factor CHD4. Genes Dev 29, 489-494, doi:10.1101/gad.256214.114 (2015).
34 Litman, R. et al. BACH1 is critical for homologous recombination and appears to be the Fanconi anemia gene product FANCJ. Cancer Cell 8, 255-265 (2005).
35 Peng, M., Litman, R., Jin, Z., Fong, G. & Cantor, S. B. BACH1 is a DNA repair protein supporting BRCA1 damage response. Oncogene 25, 2245-2253, doi:1209257 [pii]10.1038/sj.onc.1209257 (2006).
36 Peng, M. et al. The FANCJ/MutLalpha interaction is required for correction of the cross-link response in FA-J cells. Embo J 26, 3238-3249 (2007).
37 Peng, M., Xie, J., Ucher, A., Stavnezer, J. & Cantor, S. B. Crosstalk between BRCA-Fanconi anemia and mismatch repair pathways prevents MSH2-dependent aberrant DNA damage responses. The EMBO journal, doi:10.15252/embj.201387530 (2014).
38 Xie, J. et al. An MLH1 mutation links BACH1/FANCJ to colon cancer, signaling, and insight toward directed therapy. Cancer prevention research 3, 1409-1416, doi:10.1158/1940-6207.CAPR-10-0118 (2010).
39 Dash, R. C. et al. Structural Approach To Identify a Lead Scaffold That Targets the Translesion Synthesis Polymerase Revl. J Chem Inf Model 58, 2266-2277, doi:10.1021/acs.jcim.8b00535 (2018).
40 Deberardinis, A. M. et al. Structure-activity relationships for vitamin D3-based aromatic a-ring analogues as hedgehog pathway inhibitors. J Med Chem 57, 3724-3736, doi:10.1021/jm401812d (2014).
41 Hadden, M. K. Recent Advances in Developmental Signaling Pathway Inhibitors for the Treatment of Cancer. Curr Med Chem 22, 4031-4032 (2015).
42 Maschinot, C. A., Chau, L. Q., Wechsler-Reya, R. J. & Hadden, M. K. Synthesis and evaluation of third generation vitamin D3 analogues as inhibitors of Hedgehog signaling. Eur J Med Chem 162, 495-506, doi:10.1016/j.ejmech.2018.11.028 (2019).
43 Ozen, Z. et al. Small molecule scaffolds that disrupt the Rev1-CT/RIR protein-protein interaction. Bioorg Med Chem 26, 4301-4309, doi:10.1016/j.bmc.2018.07.029 (2018).
44 Pace, J. R. et al. Repurposing the Clinically Efficacious Antifungal Agent Itraconazole as an Anticancer Chemotherapeutic. J Med Chem 59, 3635-3649, doi:10.1021/acs.jmedchem.5b01718 (2016).
45 Teske, K. A. et al. Development of posaconazole-based analogues as hedgehog signaling pathway inhibitors. Eur J Med Chem 163, 320-332, doi:10.1016/j.ejmech.2018.11.056 (2019).
46 Berti, M. et al. Human RECQ1 promotes restart of replication forks reversed by DNA topoisomerase I inhibition. Nat Struct Mol Biol 20, 347-354, doi:10.1038/nsmb.2501 (2013).
47 Berti, M. & Vindigni, A. Replication stress: getting back on track. Nat Struct Mol Biol 23, 103-109, doi:10.1038/nsmb.3163 (2016).
48 Lemacon, D. et al. MREll and EXO1 nucleases degrade reversed forks and elicit MUS81-dependent fork rescue in BRCA2-deficient cells. Nature communications 8, 860, doi:10.1038/s41467-017-01180-5 (2017).
49 Pike, A. C. et al. Human RECQ1 helicase-driven DNA unwinding, annealing, and branch migration: insights from DNA complex structures. Proc Natl Acad Sci U S A 112, 4286-4291, doi:10.1073/pnas.1417594112 (2015).
50 Thangavel, S. et al. DNA2 drives processing and restart of reversed replication forks in human cells. J Cell Biol 208, 545-562, doi:10.1083/jcb.201406100 (2015).
51 Vindigni, A. & Lopes, M. Combining electron microscopy with single molecule DNA fiber approaches to study DNA replication dynamics. Biophys Chem, doi:10.1016/j.bpc.2016.11.014 (2016).
52 Peng, M. et al. Opposing Roles of FANCJ and HLTF Protect Forks and Restrain Replication during Stress. Cell Rep 24, 3251-3261, doi:10.1016/j.celrep.2018.08.065 (2018).
53 Zamborszky, J. et al. Loss of BRCA1 or BRCA2 markedly increases the rate of base substitution mutagenesis and has distinct effects on genomic deletions. Oncogene 36, 746-755, doi:10.1038/onc.2016.243 (2017).
54 Nacson, J. et al. BRCA1 Mutation-Specific Responses to 53BP1 Loss-Induced Homologous Recombination and PARP Inhibitor Resistance. Cell Rep 24, 3513-3527 e3517, doi:10.1016/j.celrep.2018.08.086 (2018).
55 George, E. et al. A patient-derived-xenograft platform to study BRCA-deficient ovarian cancers. JCI Insight 2, e89760, doi:10.1172/jci.insight.89760 (2017).
56 Wang, Y. et al. RING domain-deficient BRCA1 promotes PARP inhibitor and platinum resistance. J Clin Invest 126, 3145-3157, doi:10.1172/JCI87033 (2016).
57 Sirbu, B. M. et al. Identification of proteins at active, stalled, and collapsed replication forks using isolation of proteins on nascent DNA (iPOND) coupled with mass spectrometry. J Biol Chem 288, 31458-31467, doi:10.1074/jbc.M113.511337 (2013).
58 Schlacher, K. et al. Double-strand break repair-independent role for BRCA2 in blocking stalled replication fork degradation by MRE11. Cell 145, 529-542, doi:10.1016/j.ce11.2011.03.041 (2011).
59 Sirbu, B. M. et al. Analysis of protein dynamics at active, stalled, and collapsed replication forks. Gene Dev 25, 1320-1327 (2011).
60 Mann, M. Functional and quantitative proteomics using SILAC. Nat Rev Mol Cell Biol 7, 952-958, doi:10.1038/nrm2067 (2006).
61 Avkin, S. et al. p53 and p21 regulate error-prone DNA repair to yield a lower mutation load. Molecular cell 22, 407-413, doi:10.1016/j.molce1.2006.03.022 (2006).
62 Hendel, A. et al. PCNA ubiquitination is important, but not essential for translesion DNA synthesis in mammalian cells. PLoS genetics 7, e1002262, doi:10.1371/journal.pgen.1002262 (2011).
63 Jansen, J. G. et al. Separate domains of Revl mediate two modes of DNA damage bypass in mammalian cells. Molecular and cellular biology 29, 3113-3123 (2009).
64 Mijic, S. et al. Replication fork reversal triggers fork degradation in BRCA2-defective cells. Nature communications 8, 859, doi:10.1038/s41467-017-01164-5 (2017).
65 Sakai, W. et al. Functional restoration of BRCA2 protein by secondary BRCA2 mutations in BRCA2-mutated ovarian carcinoma. Cancer Res 69, 6381-6386, doi:0008-5472.CAN-09-1178 [pii]10.1158/0008-5472.CAN-09-1178 (2009).
66 Cox, J. & Mann, M. MaxQuant enables high peptide identification rates, individualized p.p.b.-range mass accuracies and proteome-wide protein quantification. Nat Biotechnol 26, 1367-1372, doi:10.1038/nbt.1511 (2008).
67 Phipson, B., Lee, S., Majewski, I. J., Alexander, W. S. & Smyth, G. K. Robust Hyperparameter Estimation Protects against Hypervariable Genes and Improves Power to Detect Differential Expression. Ann Appl Stat 10, 946-963, doi:10.1214/16-AOAS920 (2016).
68 Taglialatela, A. et al. Restoration of Replication Fork Stability in BRCA1- and BRCA2-Deficient Cells by Inactivation of SNF2-Family Fork Remodelers. Mol Cell 68, 414-430 e418, doi:10.1016/j.molce1.2017.09.036 (2017).
69 Kile, A. C. et al. HLTF's Ancient HIRAN Domain Binds 3′ DNA Ends to Drive Replication Fork Reversal. Mol Cell 58, 1090-1100, doi:10.1016/j.molce1.2015.05.013 (2015).
70 Lossaint, G. et al. FANCD2 binds MCM proteins and controls replisome function upon activation of s phase checkpoint signaling. Molecular cell 51, 678-690 (2013).
71 Lachaud, C. et al. Ubiquitinated Fancd2 recruits Fanl to stalled replication forks to prevent genome instability. Science 351, 846-849, doi:10.1126/science.aad5634 (2016).
72 Haracska, L., Unk, I., Prakash, L. & Prakash, S. Ubiquitylation of yeast proliferating cell nuclear antigen and its implications for translesion DNA synthesis. Proc Natl Acad Sci U S A 103, 6477-6482 (2006).
73 Petruk, S. et al. TrxG and PcG proteins but not methylated histones remain associated with DNA through replication. Cell 150, 922-933, doi:10.1016/j.ce11.2012.06.046 (2012).
74 Kolinjivadi, A. M. et al. Smarcall-Mediated Fork Reversal Triggers Mre11-Dependent Degradation of Nascent DNA in the Absence of Brca2 and Stable Rad51 Nucleofilaments. Mol Cell 67, 867-881 e867, doi:10.1016/j.molce1.2017.07.001 (2017).
75 Eelen, G. et al. Expression of the BRCA1-interacting protein Bripl/BACH1/FANCJ is driven by E2F and correlates with human breast cancer malignancy. Oncogene 27, 4233-4241 (2008).
76 Gupta, I. et al. BRIP1 overexpression is correlated with clinical features and survival outcome of luminal breast cancer subtypes. Endocr Connect 7, 65-77, doi:10.1530/EC-17-0173 (2018).
77 Hampton, O. A. et al. A sequence-level map of chromosomal breakpoints in the MCF-7 breast cancer cell line yields insights into the evolution of a cancer genome. Genome Res 19, 167-177, doi:10.1101/gr.080259.108 (2009).
78 Cantor, S. B. & Calvo, J. A. Fork Protection and Therapy Resistance in Hereditary Breast Cancer. Cold Spring Harb Symp Quant Biol, doi:10.1101/sqb.2017.82.034413 (2018).
79 Meghani, K. et al. Multifaceted Impact of MicroRNA 493-5p on Genome-Stabilizing Pathways Induces Platinum and PARP Inhibitor Resistance in BRCA2-Mutated Carcinomas. Cell Rep 23, 100-111, doi:10.1016/j.celrep.2018.03.038 (2018).
80 Rondinelli, B. et al. EZH2 promotes degradation of stalled replication forks by recruiting MUS81 through histone H3 trimethylation. Nat Cell Biol 19, 1371-1378, doi:10.1038/ncb3626 (2017).
81 Kotsantis, P., Petermann, E. & Boulton, S. J. Mechanisms of Oncogene-Induced Replication Stress: Jigsaw Falling into Place. Cancer Discov 8, 537-555, doi:10.1158/2159-8290.CD-17-1461 (2018).
82 Xie, K., Doles, J., Hemann, M. T. & Walker, G. C. Error-prone translesion synthesis mediates acquired chemoresistance. P Natl Acad Sci USA 107, 20792-20797, doi:10.1073/pnas.1011412107 (2010).
83 Doles, J. et al. Suppression of Rev3, the catalytic subunit of Pol{zeta}, sensitizes drug-resistant lung tumors to chemotherapy. P Natl Acad Sci USA 107, 20786-20791 (2010).
84 Ryan, A. Azoreductases in drug metabolism. Br J Pharmacol 174, 2161-2173, doi:10.1111/bph.13571 (2017).
85 Vesela, E., Chroma, K., Turi, Z. & Mistrik, M. Common Chemical Inductors of Replication Stress: Focus on Cell-Based Studies. Biomolecules 7, doi:10.3390/biom7010019 (2017).

VI. PARPi Synthetic Lethality Derives from Replication-Associated ssDNA Gaps

BRCA1 or BRCA2 (BRCA)-deficient tumor cells have defects in DNA double strand break repair that are thought to underlie the sensitivity to poly(ADP-ribose) polymerase inhibitor (PARPi). Given the recent finding that PARPi initially accelerates DNA replication, it was proposed that high speed DNA replication ultimately causes DNA breaks that drive synthetic lethality in BRCA deficient cells. Here, an alternative hypothesis is tested that PARPi sensitivity results from combined replication dysfunction causing a toxic accumulation of replication-associated single-stranded DNA (ssDNA) gaps. Consistent with this interpretation, PARPi treatment induces ssDNA gaps in replication tracts that are increased in BRCA deficient cells and avoided in FANCJ deficient cells that are not sensitive to PARPi. Furthermore, it is herein demonstrated that ssDNA gap suppression underlies known and de novo models of PARPi resistance in BRCA deficient cell lines and tumor samples. Collectively, a molecular link between PARPi sensitivity and ssDNA gaps may improve the understanding of synthetic lethal interactions in BRCA cancer and permit the identification of chemosensitive tumors, as well as suggesting potential therapies that target ssDNA gap suppression pathways.

BRCA1- and BRCA2-deficient cancer cells have a synthetic lethal interaction with PARP inhibition^{1,2}. This finding has led to the use of several PARP inhibitors (PARPi) in the clinic to treat BRCA deficient and other cancers³⁻⁵. The synthetic lethality is thought to derive from PARPi inducing DNA double strand breaks that persist in BRCA deficient cells due to defects in homologous recombination (HR)^{1,2}. BRCA deficient cancer cells are also defective in shielding stalled DNA replication forks from nuclease degradation due to defects in fork protection (FP)^{6,7}. Unprotected forks are thought to ultimately collapse into broken forks that further sensitizes to PARPi. Thus, a loss of HR and/or FP have been proposed to sensitize BRCA deficient cells to PARPi as well as other genotoxins such as cisplatin.

Consistent with this model, restoration of HR or FP can promote chemoresistance to these agents⁸⁻¹². However, this model of therapy response is at odds with recent reports that indicate that these chemotoxic agents do not initially induce DNA breaks or even pause DNA replication forks¹³⁻¹⁵. Indeed, PARPi accelerates DNA replication¹⁶. To square this finding with the framework that HR and/or FP defects causes the toxicity to PARPi, it was proposed that high speed DNA replication ultimately causes massive replication stress that leads to fork collapse and DNA breaks^{16,17}. Understandably, identifying the sensitizing lesion may provide information to effectively design and improve chemotherapies as well as for targeting chemoresistance mechanisms.

The data provided herein shows that the genotoxic lesion driving PARPi synthetic lethality in BRCA deficient cancer may be due to wide-spread single-stranded DNA (ssDNA) gaps. In support of this model, ssDNA gaps accumulate following PARP inhibitors, potentially reflecting the role of PARP1 in the repair of ssDNA breaks or processing Okazaki fragments^{16,18-20}. ssDNA gaps could also result from loss of PARP1 in the regulation of replication-fork reversal—a mechanism by which replication forks reverse direction when confronted with replication obstacles²¹⁻²³. Indeed, defects in fork reversal due to loss of fork remodelers generates replication-associated ssDNA gaps²⁴. Yeast cells devoid of PARPs do not undergo reversal and accumulate long ssDNA stretches at both fork junctions and in post-replicative regions^{25,26}. Deficiencies in the recombination protein, RAD51, has been reported to lead to replication stress induced ssDNA gaps, a phenomenon that also derives from deficiency in BRCA1 or BRCA2^{14,27,28,29}. Consequently, as shown here, PARPi-induced ssDNA gaps are elevated in BRCA deficient cells. Indicating that ssDNA gaps are fundamental to therapy response, ssDNA gaps were observed to be suppressed in PARPi resistant BRCA deficient cells. Collectively, these findings highlight the importance of considering an ssDNA gap toxicity threshold in chemotherapy response and as a biomarker of tumor response to chemotherapy.

A. PARPi Generates ssDNA Gaps that are Elevated in BRCA-Deficient Cells

To test the hypothesis that PARPi generates ssDNA gaps that are elevated in BRCA deficient cells, the response of DNA replication forks to PARPi was analyed by employing DNA fiber assays. DNA fiber assays monitor replication by incorporating nucleotide analogs into newly-synthesized DNA strands which can then be fluorescently labeled. As such, any replication perturbation can be observed with single-molecule resolution.

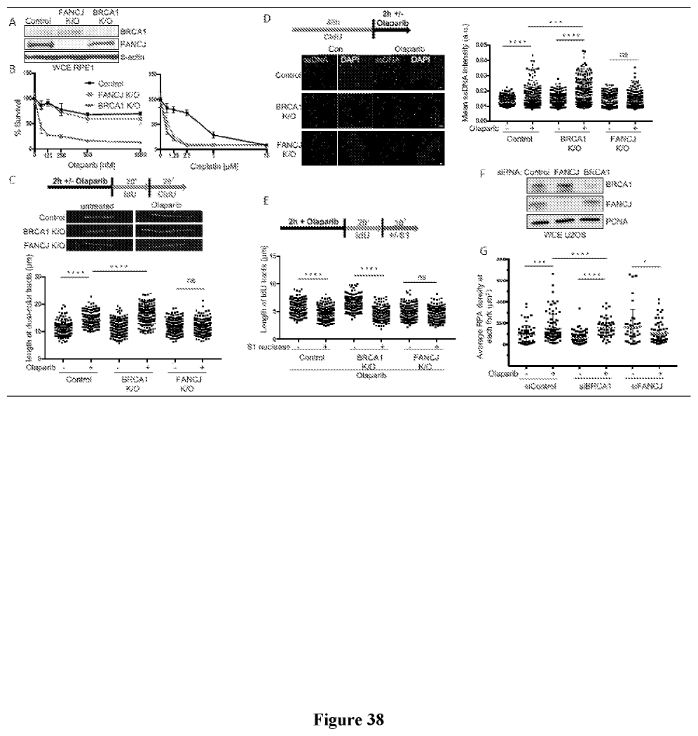

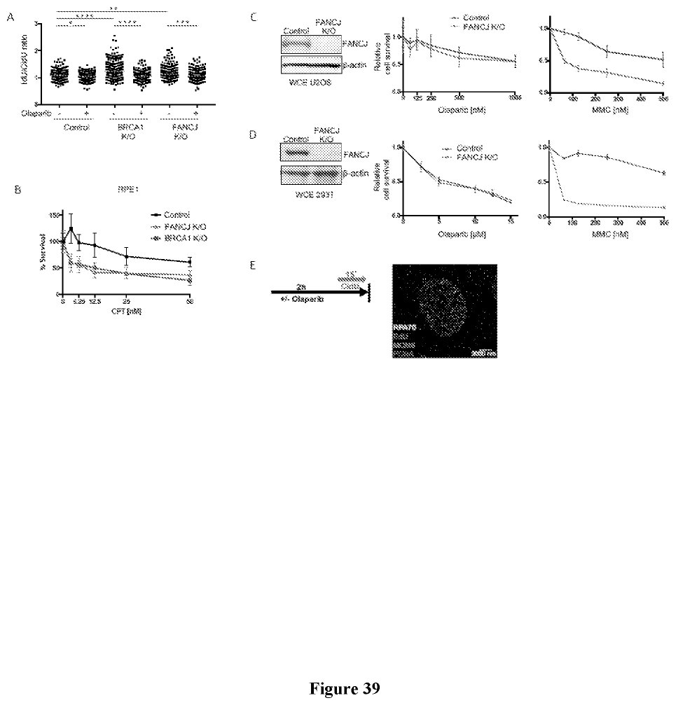

To determine if PARPi creates ssDNA replication gaps, an isogenic cell system was employed in which BRCA1 was either proficient or deleted by CRISPR-Cas9 in retinal pigment epithelial 1 (RPE1) ce11s30. As expected the BRCA1 K/O RPE1 cells were sensitive to both the PARPi, olaparib, and the DNA interstrand crosslinking (ICL) agent, cisplatin. See, FIGS. 38A and 38B. DNA fiber analysis revealed that following 2-hour PARPi treatment, both control and the BRCA1 K/O RPE1 cells displayed longer dual labeled replication tracts (5-Iodo-2′-deoxyuridine (IdU) and 5-chloro-2′-deoxyuridine (CldU)) as compared to untreated. See, FIG. 38C. Moreover, it was confirmed that BRCA1 deficiency lead to greater replication fork asymmetry that was reduced with PARPi¹⁶. See, FIG. 39A. It was also found that the BRCA1 K/O cells had longer dual labelled tracks as compared to control cells. See, FIG. 38C. Collectively these findings suggest that PARPi treatment causes replication acceleration that is exacerbated in BRCA1 deficient cells.

Next, it was determined as to whether accelerated replication leads to replication gaps in BRCA1 deficient cells. In particular, whether PARPi treatment induces ssDNA gaps. First, a non-denaturing immunofluorescence was performed on cells pre-labeled with thymidine analog, in which positive antibody stain indicates the presence of genome-wide immuno-reactive ssDNA gaps. This analysis revealed the formation of ssDNA gaps following PARPi treatment, which showed significant increase in BRCA1 K/O RPE1 cells. See, FIG. 38D. Second, to address whether the PARPi-induced gaps were in the vicinity of the accelerated replication fork, following treatment of cells with PARPi, cells were incubated with the S1 nuclease that digests ssDNA regions^{31,32}. If nascent ssDNA regions are within the labelled replication tracts, S1 nuclease will cut and therefore, shorten the visible CldU replication tract. Indeed, following PARPi treatment, labelled nascent DNA tracks appeared shorter for both control and BRCA1 K/O cells when treated with the nuclease. See, FIG. 38E Given that PARPi induces longer initial tract lengths in the BRCA1 deficient cells as compared to control, the similar reduction of tracts lengths following S1 nuclease treatment indicates that PARPi generates a more extensive region of gaps in the BRCA1 K/O cells. To further query the relationship of ssDNA gaps to individual replication assemblies, a single-molecule localization microscopy (STORM) assay was employed for direct visualization and quantification of individual replisomes (EdU, MCM6 and PCNA positive sites) and ssDNA bound RPA in the osteosarcoma cell line U2OS. See, FIG. 39B. To measure the amounts of incorporated EdU and RPA within individual replication domains, an unbiased correlation was performed based image analyses in control and BRCA1 depleted U2OS cells following PARPi treatment. See, FIGS. 38F and 38G. As compared to the control, the average RPA fork density was greater in the BRCA1 deficient U2OS cells. Collectively, these findings indicate that PARPi treatment causes gaps at replication forks. Given that these ssDNA gaps are more pronounced in PARPi sensitive BRCA1 deficient cells, ssDNA gaps are a likley cause of toxicity.

B. Fork Acceleration Alone Does Not Underlie PARPi Synthetic Lethality

It was previously reported that unrestrained replication and ssDNA gaps caused by loss of a fork remodeler, HLTF, were dependent on the BRCA1-associated helicase FANCJ (BACH1/BRIP1), which similar to BRCA1 is a hereditary breast/ovarian cancer and Fanconi anemia gene^{24,33-37}. Thus, it was determined whether PARPi induced accelerated replication and ssDNA gaps were also dependent on FANCJ. To analyze the PARPi response in an isogenic system, FANCJ K/O RPE1 cells were created. See, FIG. 38A. Similar to the BRCA1 K/O, the FANCJ K/O were sensitive to cisplatin or the double strand break inducing agent, CPT as compared to control RPE1 cells. See, FIG. 38B and FIG. 39C. Moreover, tract lengths were similar in the untreated control, BRCA1 K/O or FANCJ K/O RPE1 cell lines. However, in contrast to BRCA1 K/O cells, following PARPi treatment, FANCJ K/O cells did not display PARPi sensitivity, fork acceleration, genomic ssDNA accumulation, undergo S1 cutting, or develop RPA rich fork regions. See, FIGS. 38C-38G. These findings were not limited to RPE1 cells as FANCJ K/O U2OS and human epithelial kidney (HEK) 293T cells were also not sensitive to PARPi, but were sensitive to ICL-inducing agents.³⁵ See, FIGS. 39D and 39E. These findings indicate that PARPi causes replication acceleration and gaps in a manner dependent on FANCJ. Moreover, given that similar to BRCA1 loss, FANCJ loss also disrupts HR and FP^{24,35,38-40}. Consequently, these findings highlight that ssDNA gaps, as opposed to persistent DNA breaks at forks, are believed to underlie PARPi sensitivity.

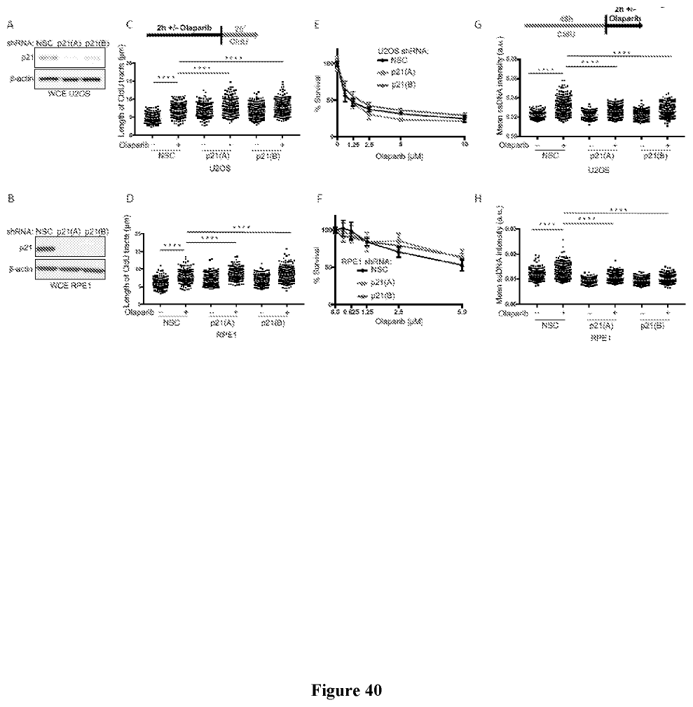

High replication fork speed and subsequent DNA breaks have been proposed to cause PARPi sensitivity in BRCA deficient cells¹⁶. One way to violate the potential threshold speed of replication is to deplete the cell cycle regulator, p21. When p21 depletion is combined with PARPi, replication forks are dramatically accelerated in U2OS cells as well as RPE1 cells. See, FIGS. 40A-40D. However, PARPi sensitivity was not elevated and ssDNA was decreased. See, FIG. 40E-40H. These findings suggest that enhanced fork speed alone is not the cause of PARPi sensitivity.

C. Gap Suppression Occurs in Distinct Models of De Novo PARPi Resistance

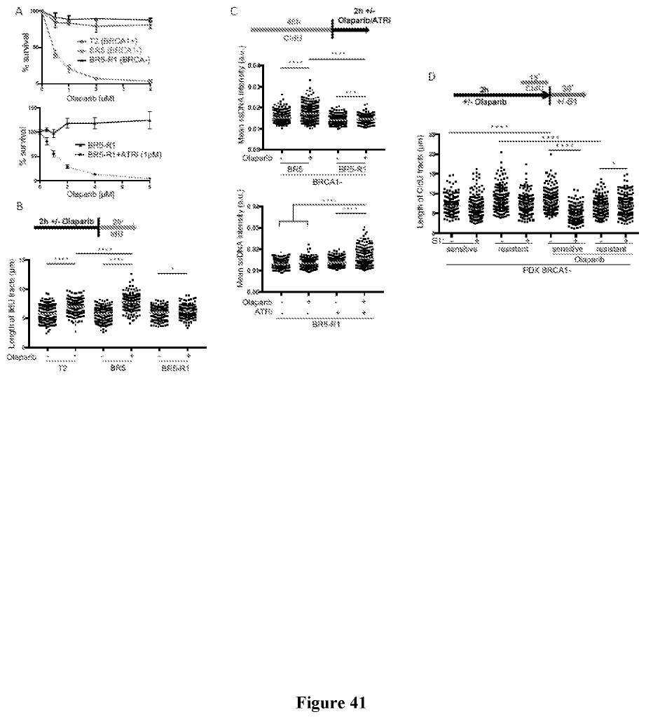

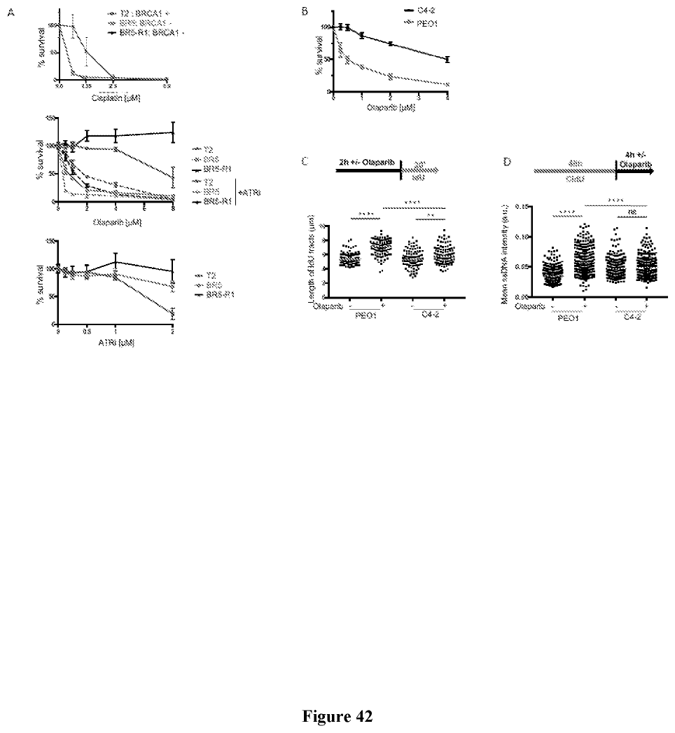

To determine whether gap suppression mechanisms occur in models of de novo PARPi resistance, cell lines were analyzed that were initially sensitive to PARPi and then had gained PARPi resistance while propagated in tissue culture⁴¹. BRCA1-deficient mouse ovarian tumor cell line, BR5, was utilized and its derived PARPi-resistant cell line, BR5-R1⁴¹. PARPi sensitivity of BR5 was confirmed as compared to BR5-R1 and BRCA1 proficient T2 cells. See, FIG. 41A. It was observed that PARPi treatment induced greater replication tract lengthening and gap induction in BRCA1 deficient BR5 cells as compared to BRCA1 proficient T2 cells. See, FIG. 41B. In contrast, PARPi resistant BR5-R1 cells did not gain longer tracts or develop gaps in response to PARPi. See, FIGS. 41B and 41C. Thus, BR5-R1 cells resemble FANCJ deficient cells, in that they both lack PARPi induced-sensitivity, lengthening, or gaps, but remain sensitive to cisplatin. See, FIG. 42A. It was also confirmed that co-incubating the BR5-R1 cell line with the ATR inhibitor (ATRi), VE-821, re-established PARPi sensitivity and noted gap induction exceeded PARPi or ATRi treatment alone, indicating that ATRi restores not only PARPi sensitivity, but also gap induction. See, FIGS. 41A, 41C and 42A.

The gain of chemotherapy resistance in tissue culture, and in patients, has been reported to result from BRCA reversion mutations that re-instate protein function in HR^{8,9}. In particular, the chemotherapy sensitive BRCA2 deficient ovarian cancer cell line, PEO1, was shown to undergo a BRCA2 reversion mutation to generate the C4-2 cell clone with functional BRCA2, HR activity, and cisplatin and PARPi resistance.^{42,43} See, FIG. 42B. It was also observed that in comparison to the PEO1 cells, the C4-2 cells maintained greater fork restraint and gained fewer gaps following PARPi treatment. See, FIGS. 42C and 42D. These findings indicate that the BRCA2 reversion mutation restores BRCA2 function not only in HR, but also in fork restraint and gap suppression.

Patient derived xenografts (PDX) were analyzed showing an initial sensitive to PARPi, but gained chemoresistance while propagated upon serial passage in mice. Tumor samples that were PARPi sensitive showed significant PARPi-induced lengthening of replication tracts. Consistent with this lengthening reflecting replication with gaps, elongated tracts dramatically shortened upon S1-nuclease treatment. In contrast, PARPi resistant tumor samples did not show PARPi-induced lengthening or S1 nuclease shortening, indicating that gaps were not present. See, FIG. 41D. Collectively, these data showing acquired PARPi resistance demonstrate a block to PARPi-induced acceleration that causes ssDNA gaps.

D. ssDNA Gaps Predict PARPi Response Better than HR or FP Status

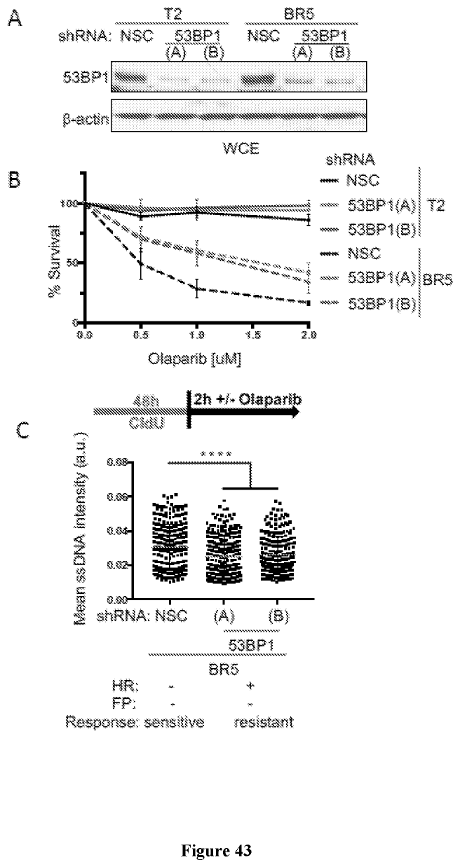

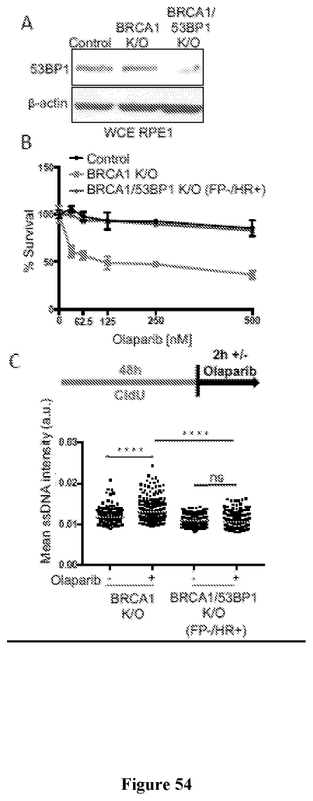

To further consider the relationship between ssDNA gaps and PARPi response, models were analyzed that vary with respect to HR, FP, and PARPi response. HR and PARPi resistance are re-established in BRCA1 deficient cells by loss of the DNA repair protein 53BP1, which limits HR by blocking DNA end resection^{10,11} . It was confirmed that 53BP1 deletion in BRCA1 K/O RPE1 cells or 53BP1 depletion in BRCA1 deficient BR5 cells enhances PARPi resistance.³⁰ See, FIG. 54A, 54B, 43A and 43B. As compared to BRCA1 deficiency alone, loss of both BRCA1 and 53BP1 reduced PARPi-induced gaps. See, FIG. 54C and 43C. These data suggest that 53BP1 loss restores not only HR, but also suppresses ssDNA gaps.

While HR remains defective, FP and PARPi resistance are re-established in BRCA2 deficient cells by loss of the chromatin remodeler, CHD4^{43,44}. Notably, CHD4 depletion not only enhanced PARPi resistance, but also suppressed PARPi-induced gaps as compared to control. Moreover, while FP remains defective, HR and PARPi resistance are re-established in BRCA2 mutant Chinese hamster cell line (V-C8) when complemented with the C-terminal BRCA2^{S3291A} mutant⁶. It was observed that the mutant, similar to wild-type expression, conferred PARPi resistance as well as ssDNA gap suppression.

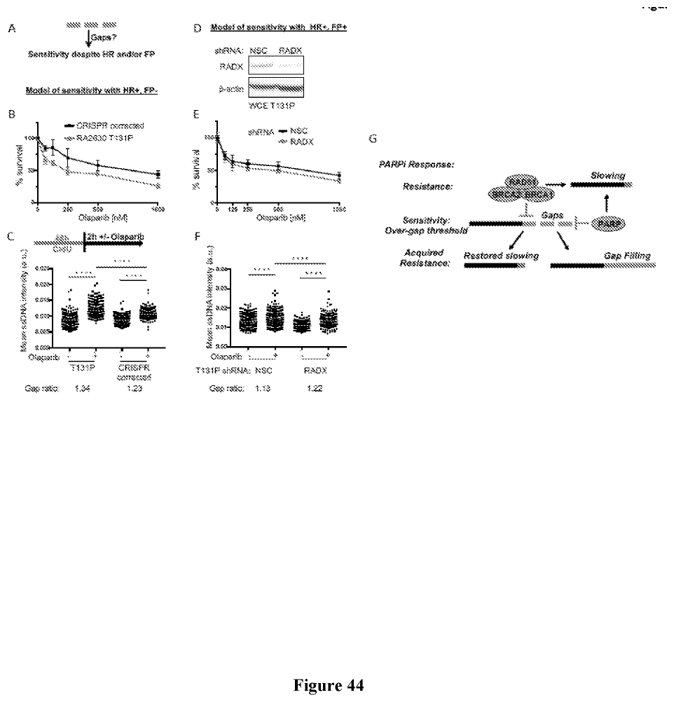

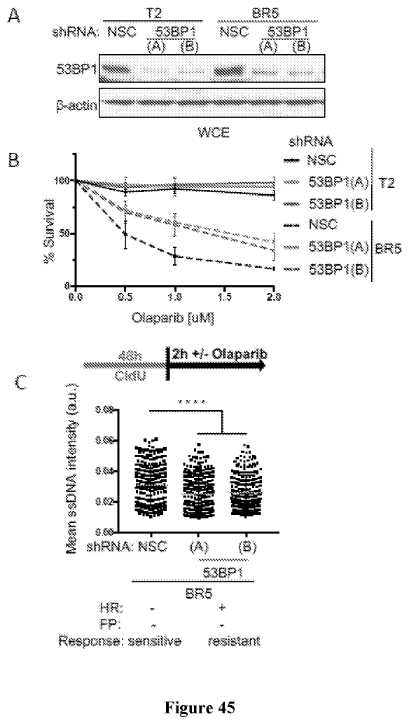

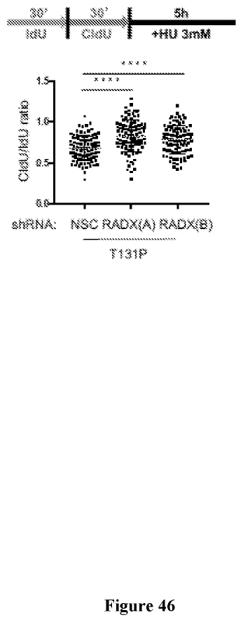

To better understand the link between gaps and therapy response without the HR and FP confounding the interpretation of resistance mechanism, a model system is proposed in which despite HR and/or FP proficiency, cells remain PARPi sensitive allowing one to address if gaps remain. See, FIG. 44A. HR is intact in the Fanconi anemia (FA) patient fibroblast cell line that has one wild-type and one mutant (T131P) RAD51 allele, but they remain therapy sensitive as compared to a CRISPR corrected line. See, FIG. 44B; Wang et al., (2015). While PARPi-induced gaps were observed in both wild-type and mutant cells, the mutant cell line had a higher level of gap induction and ratio of gaps. See, FIG. 44C. Conceivably, the FP defect in the FA cells could generate DSBs that overwhelm HR and sensitize to PARPi. FP can be restored in the RAD51 mutant FA cells by depletion of the RAD51 negative regulator, RADX. Bhat et al., (2018). Notably, despite restored FP upon RADX depletion, the RAD51 mutant FA cells remained PARPi sensitive. See, FIGS. 44 D & 44E. Because PARPi gaps are observed and the gap ratio was elevated, these findings suggest that HR and/or FP are insufficient for PARPi resistance when gaps exceed a toxicity threshold. See, FIG. 44F. Conceivably, in the models described above, HR or FP could underlie a mechanism of PARPi resistance. Moreover, residual HR or FP activity could underlie PARPi resistance in FANCJ null cells. Thus, it was sought to define a model system in which cells remain PARPi sensitive despite proficiency in HR and/or FP. Such a model system would allow one to address whether sensitivity correlates with the presence of gaps. For example, HR is intact in the Fanconi anemia (FA) patient fibroblast cell line that has one wild-type and one mutant (T131P) RAD51 allele, but they remain therapy sensitive as we confirmed as compared to a wild-type corrected line.⁴⁵ See, FIG. 46. While PARPi-induced gaps were observed in both wild-type and mutant cells, the mutant cell line had a higher level of gap induction. See, FIG. 43E. Most notably, when FP was restored by depletion of the RAD51 negative regulator, RADX,⁴⁶ the HR and FP FA cells remained PARPi sensitive and displayed PARPi-induced gaps. See, FIGS. 44J-44L and 43F. These findings suggest that HR and FP are not sufficient for PARPi resistance when ssDNA gaps exceed a toxicity threshold.

E. Summary

In the above tested model systems, HR or FP sometimes correlate with PARPi resistance whereas ssDNA gap suppression always correlates. Moreover, defects in HR or FP sometimes correlate with PARPi sensitivity whereas gaps always correlate. See Table 4.

TABLE 4
Correlations Of ssDNA gabs with PARPi Chemosensivity

| | | | | PARPi |
|---|---|---|---|---|
| Cell line | HR* | FP* | GS* | response |
| BRCA1/2 proficient | + | + | + | resistant |
| BRCA1 and 53BP1 deficient | + | − | + | resistant |
| FANCJ deficient | − | − | + | resistant |
| BRCA2 and CHD4 deficient | − | + | + | resistant |
| BRCA2 S3291A mutant | + | − | + | resistant |
| BRCA1/2 deficient | − | − | − | sensitive |
| RAD51 T131P mutant | + | − | − | sensitive |
| RAD51 T131P mutant | + | + | − | sensitive |
| and RADX deficient | | | | |

*HR: homologous recombination,
FP: fork protection,
GS: gap suppression

Together, these findings reveal that gaps as opposed to loss of HR or FP proficiency more directly predicts PARPi response. See, FIG. 44M.

PARP1 function and loss-of-function has been linked to ssDNA^{16,19,47,48}. However, ssDNA has been overlooked as the sensitizing lesion in BRCA deficient cells in favor of DNA double stranded breaks by numerous studies. Challenging this break-induced model of therapy sensitivity, PARPi do not initially generate DNA breaks or pause DNA replication forks.

Instead, PARPi dysregulates DNA replication as fork reversal and restraint are disrupted^{14,16,21-23} Therefore, a mechanism of action for PARPi-induced killing of BRCA deficient cells was in question. As clinical interventions for PARPi resistance are lacking, an understanding of the sensitizing lesion was also lacking.

The data presented herein shows that PARPi leads to dysregulated replication with the induction of ssDNA gaps that accumulate more excessively in BRCA deficient immortalized cells, human and mouse cancer cell lines, and patient tumors. Moreover, PARPi-induced gaps are restricted in FANCJ deficient cells that are not sensitive to PARPi. Further indicating gaps as a sensitizing lesion, in BRCA1 deficient cells PARPi-induced gaps are suppressed in both de novo and genetic models of PARPi resistance. Moreover, gap characterization studies using models of restored or defective HR or FP; despite differences in proficiency, revealed sensitivity if ssDNA gaps were present and resistance if ssDNA gaps were suppressed. These data also highlight that dysregulated or accelerated replication is most likely not involved in PARPi sensitivity.

Rather, these findings indicate that toxicity results from ssDNA gaps that exceed a safe threshold. It was envisioned that a toxicity threshold is more readily exceeded in BRCA deficient cells due to intrinsic defects in coordinating RAD51 replication restraint and gap avoidance functions. Indeed, it is foreseeable that replication restraint is a function of the BRCA pathway given that stress-resistant DNA synthesis is reminiscent of radioresistant DNA synthesis originally described in ATM deficient cells^{5,49}. In cells defective in restraining replication in response to stress, PARPi treatment could lead to unrestrained replication that proceeds until replication forks confront PARP-trapped proteins or other intrinsic obstacles that physically block the pre-existing replication fork. ssDNA gaps could form as replication re-initiates ahead of the block by re-priming replication. The extensive regions of under-replication could drive RPA exhaustion and replication catastrophe⁵⁰. Alternatively, given that ssDNA marks apoptotic cells and ssDNA induces apoptosis⁵¹⁻⁵⁵, the accumulation of an excessive amount of ssDNA could be the single driver of cell killing.

These findings also indicate that PARPi resistance involves ssDNA gap suppression by either a block to re-priming or by the acquisition of gap filling activity such as mediated by translesion synthesis (TLS). In support of this latter point, PARPi resistance in BRCA2 deficient cells is achieved by loss of CHD4 that elevates TLS43. Moreover, p21 is a TLS inhibitor and its loss lengthens tracts without gaps⁵⁶. Notably, 53BP1 loss uniquely rescues BRCA1 deficient cells and CHD4 loss uniquely rescues BRCA2 deficient cells^{10,11,43,44}. Thus, a mechanism of ssDNA gap suppression is distinct in BRCA backgrounds for reasons that remain to be fully understood.

These findings alos indicate that HR is not sufficient to confer PARPi resistance if ssDNA gaps are present. A greater correlation is demonstrated between gap suppression and therapy resistance than with restored HR or FP. The literature also provides several examples in which FP is not sufficient to confer resistance to PARPi. In particular, RADX depletion in BRCA1 deficient cells restores FP, but does not confer PARPi resistance⁴⁶. Likewise, it is shown herein that RADX depletion and restored FP in HR proficient FA cells is not sufficient to restore PARPi. Indeed, a rationale for limiting PARPi to HR defective cancers is already in question as recent clinical trials found significant clinical benefit across ovarian cancer patients regardless of BRCA status⁵. These findings indicate that HR deficiency may not be a root cause of chemotherapy response or for that matter “BRACAness”. Collectively, these findings highlight the importance of considering ssDNA gaps as a potential biomarker and determinant of chemotherapy response.

### REFERENCES: SECTION VI

1 Farmer, H. et al. Targeting the DNA repair defect in BRCA mutant cells as a therapeutic strategy. Nature 434, 917-921 (2005).
2 Bryant, H. E. et al. Specific killing of BRCA2-deficient tumours with inhibitors of poly(ADP-ribose) polymerase. Nature 434, 913-917 (2005).
3 Lord, C. J., Tutt, A. N. & Ashworth, A. Synthetic Lethality and Cancer Therapy: Lessons Learned from the Development of PARP Inhibitors. Annual review of medicine, doi:10.1146/annurev-med-050913-022545 (2014).
4 Mirza, M. R., Pignata, S. & Ledermann, J. A. Latest clinical evidence and further development of PARP inhibitors in ovarian cancer. Ann Oncol 29, 1366-1376, doi:10.1093/annonc/mdyl74 (2018).
5 Ashworth, A. & Lord, C. J. Synthetic lethal therapies for cancer: what's next after PARP inhibitors? Nat Rev Clin Oncol 15, 564-576, doi:10.1038/s41571-018-0055-6 (2018).
6 Schlacher, K. et al. Double-strand break repair-independent role for BRCA2 in blocking stalled replication fork degradation by MRE11. Cell 145, 529-542, doi:10.1016/j.ce11.2011.03.041 (2011).
7 Schlacher, K., Wu, H. & Jasin, M. A distinct replication fork protection pathway connects Fanconi anemia tumor suppressors to RAD51-BRCA1/2. Cancer cell 22, 106-116 (2012).
8 Sakai, W. et al. Secondary mutations as a mechanism of cisplatin resistance in BRCA2-mutated cancers. Nature 451, 1116-1120, doi:nature06633 [pii]10.1038/nature06633 (2008).
9 Edwards, S. L. et al. Resistance to therapy caused by intragenic deletion in BRCA2. Nature 451, 1111-1115 (2008).
10 Bouwman, P. et al. 53BP1 loss rescues BRCA1 deficiency and is associated with triple-negative and BRCA-mutated breast cancers. Nature structural & molecular biology 17, 688-695, doi:10.1038/nsmb.1831 (2010).
11 Bunting, S. F. et al. 53BP1 inhibits homologous recombination in Brcal-deficient cells by blocking resection of DNA breaks. Cell 141, 243-254, doi:10.1016/j.ce11.2010.03.012 (2010).
12 Chaudhuri, A. R. et al. Replication fork stability confers chemoresistance in BRCA-deficient cells. Nature 535, 382-387, doi:10.1038/nature18325 (2016).
13 Huang, J. et al. The DNA translocase FANCM/MHF promotes replication traverse of DNA interstrand crosslinks. Molecular cell 52, 434-446 (2013).
14 Zellweger, R. et al. Rad51-mediated replication fork reversal is a global response to genotoxic treatments in human cells. J Cell Biol 208, 563-579, doi:10.1083/jcb.201406099 (2015).
15 Mutreja, K. et al. ATR-Mediated Global Fork Slowing and Reversal Assist Fork Traverse and Prevent Chromosomal Breakage at DNA Interstrand Cross-Links. Cell Rep 24, 2629-2642 e2625, doi:10.1016/j.celrep.2018.08.019 (2018).
16 Maya-Mendoza, A. et al. High speed of fork progression induces DNA replication stress and genomic instability. Nature 559, 279-284, doi:10.1038/s41586-018-0261-5 (2018).
17 Quinet, A. & Vindigni, A. Superfast DNA replication causes damage in cancer cells. Nature 559, 186-187, doi:10.1038/d41586-018-05501-6 (2018).
18 Caldecott, K. W., Aoufouchi, S., Johnson, P. & Shall, S. XRCC1 polypeptide interacts with DNA polymerase beta and possibly poly (ADP-ribose) polymerase, and DNA ligase III is a novel molecular ‘nick-sensor’ in vitro. Nucleic Acids Res 24, 4387-4394, doi:10.1093/nar/24.22.4387 (1996).
19 Leppard, J. B., Dong, Z., Mackey, Z. B. & Tomkinson, A. E. Physical and functional interaction between DNA ligase IIIalpha and poly(ADP-Ribose) polymerase 1 in DNA single-strand break repair. Mol Cell Biol 23, 5919-5927, doi:10.1128/mcb.23.16.5919-5927.2003 (2003).
20 Hanzlikova, H. et al. The Importance of Poly(ADP-Ribose) Polymerase as a Sensor of Unligated Okazaki Fragments during DNA Replication. Mol Cell 71, 319-331 e313, doi:10.1016/j.molce1.2018.06.004 (2018).
21 Sugimura, K., Takebayashi, S., Taguchi, H., Takeda, S. & Okumura, K. PARP-1 ensures regulation of replication fork progression by homologous recombination on damaged DNA. J Cell Biol 183, 1203-1212, doi:10.1083/jcb.200806068 (2008).
22 Ray Chaudhuri, A. et al. Topoisomerase I poisoning results in PARP-mediated replication fork reversal. Nat Struct Mol Biol 19, 417-423, doi:10.1038/nsmb.2258 (2012).
23 Berti, M. et al. Human RECQ1 promotes restart of replication forks reversed by DNA topoisomerase I inhibition. Nat Struct Mol Biol 20, 347-354, doi:10.1038/nsmb.2501 (2013).
24 Peng, M. et al. Opposing Roles of FANCJ and HLTF Protect Forks and Restrain Replication during Stress. Cell Rep 24, 3251-3261, doi:10.1016/j.celrep.2018.08.065 (2018).
25 Sogo, J. M., Lopes, M. & Foiani, M. Fork reversal and ssDNA accumulation at stalled replication forks owing to checkpoint defects. Science 297, 599-602 (2002).
26 Lopes, M., Foiani, M. & Sogo, J. M. Multiple mechanisms control chromosome integrity after replication fork uncoupling and restart at irreparable UV lesions. Mol Cell 21, 15-27, doi:10.1016/j.molce1.2005.11.015 (2006).
27 Hashimoto, Y., Ray Chaudhuri, A., Lopes, M. & Costanzo, V. Rad51 protects nascent DNA from Mrell-dependent degradation and promotes continuous DNA synthesis. Nat Struct Mol Biol 17, 1305-1311, doi:10.1038/nsmb.1927 (2010).
28 Kolinjivadi, A. M. et al. Moonlighting at replication forks—a new life for homologous recombination proteins BRCA1, BRCA2 and RAD51. FEBS Lett 591, 1083-1100, doi:10.1002/1873-3468.12556 (2017).
29 Kolinjivadi, A. M. et al. Smarcall-Mediated Fork Reversal Triggers Mrell-Dependent Degradation of Nascent DNA in the Absence of Brca2 and Stable Rad51 Nucleofilaments. Mol Cell 67, 867-881 e867, doi:10.1016/j.molce1.2017.07.001 (2017).
30 Noordermeer, S. M. et al. The shieldin complex mediates 53BP1-dependent DNA repair. Nature 560, 117-121, doi:10.1038/s41586-018-0340-7 (2018).
31 Quinet, A. et al. Translesion synthesis mechanisms depend on the nature of DNA damage in UV-irradiated human cells. Nucleic Acids Res 44, 5717-5731, doi:10.1093/nar/gkw280 (2016).
31 Quinet, A., Carvajal-Maldonado, D., Lemacon, D. & Vindigni, A. DNA Fiber Analysis: Mind the Gap! Methods Enzymol 591, 55-82, doi:10.1016/bs.mie.2017.03.019 (2017).
33 Sawyer, S. L. et al. Biallelic mutations in BRCA1 cause a new Fanconi anemia subtype. Cancer Discov 5, 135-142, doi:10.1158/2159-8290.CD-14-1156 (2015).
34 Cantor, S. et al. The BRCA1-associated protein BACH1 is a DNA helicase targeted by clinically relevant inactivating mutations. Proc Natl Acad Sci U S A 101, 2357-2362 (2004).
35 Litman, R. et al. BACH1 is critical for homologous recombination and appears to be the Fanconi anemia gene product FANCJ. Cancer Cell 8, 255-265 (2005).
36 Levitus, M. et al. The DNA helicase BRIP1 is defective in Fanconi anemia complementation group J. Nat Genet 37, 934-935, doi:ng1625 [pii]10.1038/ng1625 (2005).
37 Levran, O. et al. The BRCA1-interacting helicase BRIP1 is deficient in Fanconi anemia. Nat Genet 37, 931-933 (2005).
38 Peng, M., Litman, R., Jin, Z., Fong, G. & Cantor, S. B. BACH1 is a DNA repair protein supporting BRCA1 damage response. Oncogene 25, 2245-2253, doi:1209257 [pii]10.1038/sj.onc.1209257 (2006).
39 Suhasini, A. N. et al. Fanconi anemia group J helicase and MRE11 nuclease interact to facilitate the DNA damage response. Molecular and cellular biology 33, 2212-2227 (2013).
40 Nath, S., Somyajit, K., Mishra, A., Scully, R. & Nagaraju, G. FANCJ helicase controls the balance between short- and long-tract gene conversions between sister chromatids. Nucleic Acids Res 45, 8886-8900, doi:10.1093/nar/gkx586 (2017).
41 Yazinski, S. A. et al. ATR inhibition disrupts rewired homologous recombination and fork protection pathways in PARP inhibitor-resistant BRCA-deficient cancer cells. Genes Dev 31, 318-332, doi:10.1101/gad.290957.116 (2017).
42 Swisher, E. M. et al. Secondary BRCA1 mutations in BRCA1-mutated ovarian carcinomas with platinum resistance. Cancer Res 68, 2581-2586, doi:68/8/2581 [pii]10.1158/0008-5472.CAN-08-0088 (2008).
43 Guillemette, S. et al. Resistance to therapy in BRCA2 mutant cells due to loss of the nucleosome remodeling factor CHD4. Genes Dev 29, 489-494, doi:10.1101/gad.256214.114 (2015).
44 Ray Chaudhuri, A. et al. Replication fork stability confers chemoresistance in BRCA-deficient cells. Nature 535, 382-387, doi:10.1038/nature18325 (2016).
45 Wang, A. T. et al. A Dominant Mutation in Human RAD51 Reveals Its Function in DNA Interstrand Crosslink Repair Independent of Homologous Recombination. Mol Cell 59, 478-490, doi:10.1016/j.molce1.2015.07.009 (2015).
46 Bhat, K. P. et al. RADX Modulates RAD51 Activity to Control Replication Fork Protection. Cell Rep 24, 538-545, doi:10.1016/j.celrep.2018.06.061 (2018).
47 Lonskaya, I. et al. Regulation of poly(ADP-ribose) polymerase-1 by DNA structure-specific binding. J Biol Chem 280, 17076-17083, doi:10.1074/jbc.M413483200 (2005).
48 Ray Chaudhuri, A. & Nussenzweig, A. The multifaceted roles of PARP1 in DNA repair and chromatin remodelling. Nat Rev Mol Cell Biol 18, 610-621, doi:10.1038/nrm.2017.53 (2017).
49 Painter, R. B. Radioresistant DNA synthesis: an intrinsic feature of ataxia telangiectasia. Mutat Res 84, 183-190 (1981).
50 Toledo, L. I. et al. ATR prohibits replication catastrophe by preventing global exhaustion of RPA. Cell 155, 1088-1103, doi:10.1016/j.ce11.2013.10.043 (2013).
51 Frankfurt, O. S., Robb, J. A., Sugarbaker, E. V. & Villa, L. Monoclonal antibody to single-stranded DNA is a specific and sensitive cellular marker of apoptosis. Exp Cell Res 226, 387-397, doi:10.1006/excr.1996.0240 (1996).
52 Chen, J. et al. Early detection of DNA strand breaks in the brain after transient focal ischemia: implications for the role of DNA damage in apoptosis and neuronal cell death. J Neurochem 69, 232-245 (1997).
53 Gagna, C. E., Kuo, H. R., Homan, C., Hammond, I. & Lambert, W. C. Use of anti-single-stranded DNA antibodies to localize and quantify denatured DNA during cell death. Cell Biol Int 24, 657-659, doi:10.1006/cbir.2000.0574 (2000).
54 Michiue, T. et al. Single-stranded DNA as an immunohistochemical marker of neuronal damage in human brain: an analysis of autopsy material with regard to the cause of death. Forensic Sci Int 178, 185-191, doi:10.1016/j.forsciint.2008.03.019 (2008).
55 Nur, E. K. A. et al. Single-stranded DNA induces ataxia telangiectasia mutant (ATM)/p53-dependent DNA damage and apoptotic signals. J Biol Chem 278, 12475-12481, doi:10.1074/jbc.M212915200 (2003).
56 Avkin, S. et al. p53 and p21 regulate error-prone DNA repair to yield a lower mutation load. Molecular cell 22, 407-413, doi:10.1016/j.molce1.2006.03.022 (2006).

VII. Replication ssDNA Gaps Underlie BRCA-Deficiency and Chemotherapy Response

Defects in DNA repair and/or the protection of stalled DNA replication forks are thought to underlie the chemosensitivity of tumors deficient in hereditary breast cancer genes (e.g., BRCA1 and BRCA2; (BRCA)). Challenging this assumption, recent findings indicate that chemotherapies such as cisplatin used to treat BRCA deficient tumors do not initially cause DNA breaks or stall forks. (Mutreja et al., 2018). The data presented herein show that single stranded DNA (ssDNA) replication gaps may underlie a hypersensitivity of BRCA deficient cancer to chemotherapy, and that defects in homologous recombination or fork protection do not.

Specifically, in BRCA deficient cells, ssDNA gaps develop because replication may not be effectively restrained in response to replication stress. ssDNA gap suppression by either restoration of fork restraint or gap filling confers therapy resistance in tissue culture and BRCA patient tumors. In contrast, restored fork protection or homologous recombination can be uncoupled from therapy resistance when gaps are present. Together, this data indicate that ssDNA replication gaps underlie the BRCA cancer phenotype, “BRCAness,” and provide a common mechanism of action for chemotherapies.

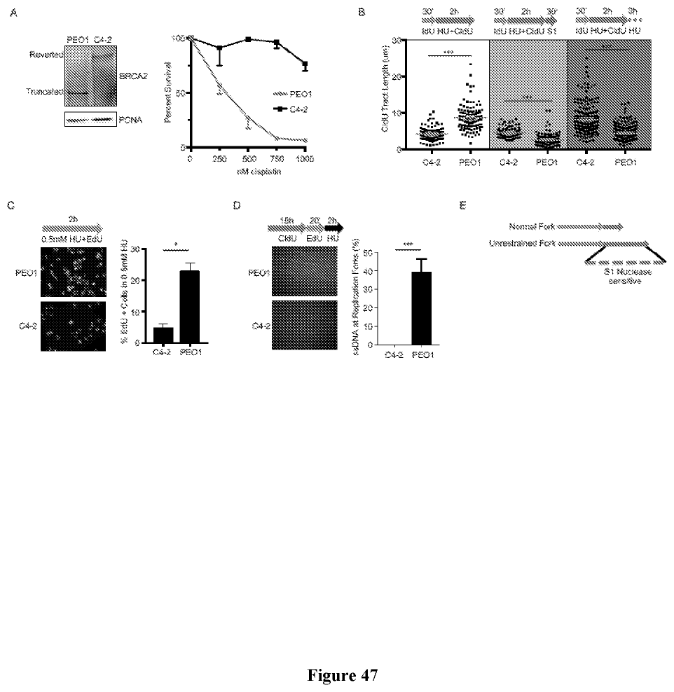

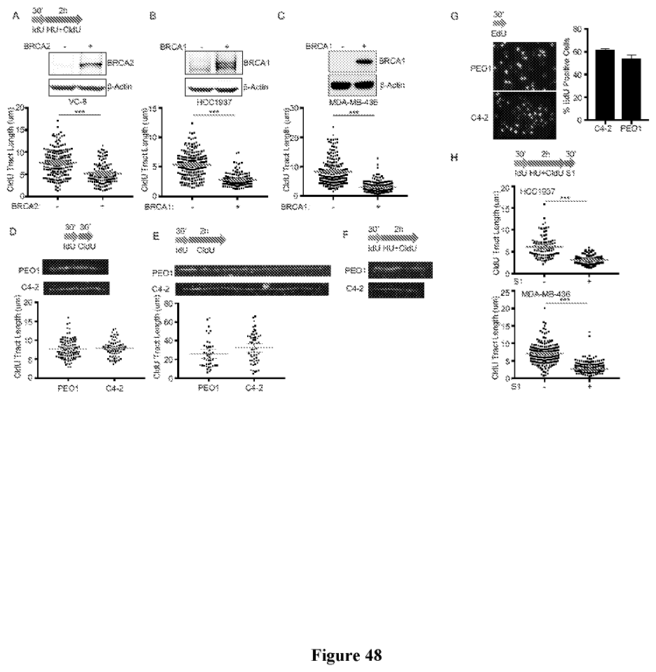

To analyze a mechanism underlying a hypersensitivity of BRCA deficient cancers to chemotherapy, the immediate response of replication to stress was monitored. A parental PEO1 cancer cell line has a truncated BRCA2 protein and is hypersensitive to cisplatin, whereas the BRCA2 proficient PEO1 reversion cell line, C4-2, is resistant to cisplatin. See, FIG. 47A. (Sakai et al., 2009). The cell lines were incubated with 5-Iodo-2′ -deoxyuridine (IdU) for 30 minutes followed by 5-chloro-2′ -deoxyuridine (CldU) for 2 h in the presence of hydroxyurea (HU). Longer CldU tracks were observed in PEO1 cells compared to C4-2 cells, indicating replication in PEO1 cells had continued during HU. See, FIG. 47B. These data suggest that BRCA2 effectively restrains replication during stress. Indeed, a BRCA2 deficient Chinese hamster cell line (VC-8) (Schlacher et al., 2011), and two BRCA1 deficient breast cancer lines (HCC1937 and MDA-MB-436) also showed improved fork restraint upon complementation with the respective full length BRCA genes. See, FIGS. 48A-48E. These data suggest that replication restraint is a function of the BRCA proteins. In agreement with fiber assays, analysis of global cellular replication based on 5-ethynyl-2′ -deoxyuridine (EdU) incorporation supported that BRCA2 restrains replication during stress. See, FIGS. 47C and FIG. 48G.

It was hypothesized that failure to fully restrain replication in response to stress in BRCA deficient cells would result in poorly replicated regions that contain ssDNA. To test this hypothesis, a DNA fiber assay was performed followed by incubation with S1 nuclease. S1 cuts at ssDNA regions and secondary DNA structures as an indicator of poor quality DNA(Quinet et al., 2017). Indeed, labelled nascent DNA tracks were S1 sensitive in PEO1 cells, but not C4-2 cells. These S1 sensitive nascent DNA regions were also degraded after continued exposure to replication stress. See, FIG. 47B. Direct analysis of ssDNA in active replication regions using a non-denaturing DNA fiber assay validated that ssDNA was present adjacent to newly replicating EdU positive regions in BRCA2 deficient PEO1 cells, but not in BRCA2 proficient C4-2 cells. FIG. 47D. Moreover, BRCA1 deficient cancer cells also displayed DNA replication tracks that were sensitive to S1 nuclease treatment. See, FIG. 48H. Thus, BRCA deficient cancer cells fail to fully restrain replication in the presence of stress, creating ssDNA regions that are degraded after additional exposure to stress. See, FIG. 47E.

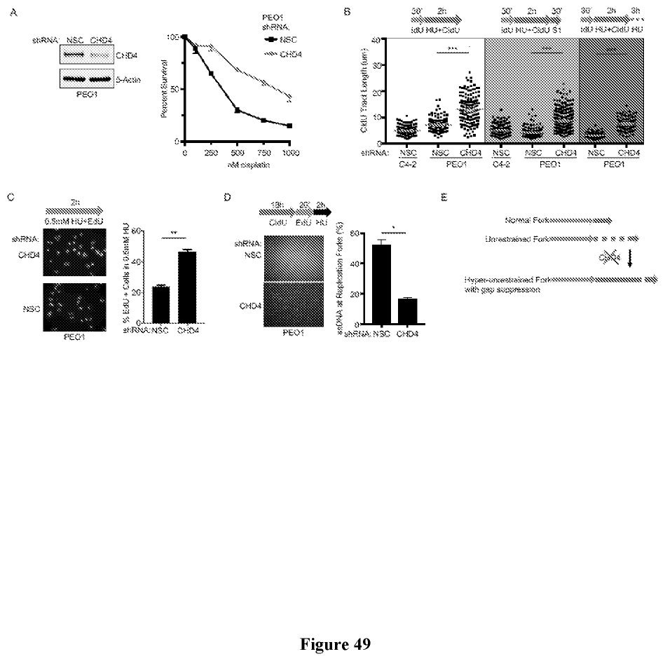

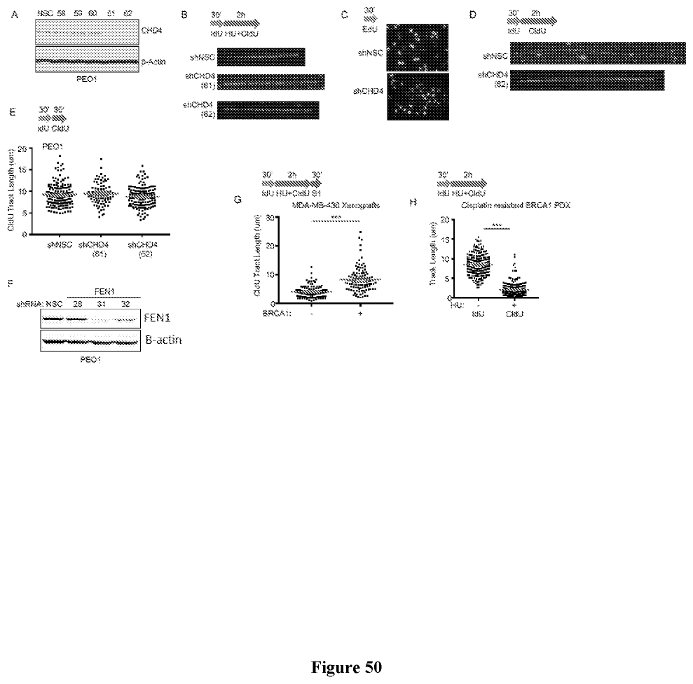

It was hypothesized that ssDNA gaps confer chemosensitivity in BRCA cancer, and that mechanisms of chemoresistance would suppress these ssDNA gaps. Indeed, ssDNA gaps were found to be suppressed when chemoresistance was achieved by depletion of the chromatin remodeling enzyme CHD4 in BRCA2 deficient PEO1 cells. See, FIG. 49A. (Guillemette et al., 2015). When CHD4 was depleted, significantly suppressed S1 nuclease sensitivity was observed as compared to the control, and nascent DNA tracks were not degraded after continued exposure to HU. See, FIG. 49B. In agreement, ssDNA gaps adjacent to regions of active replication were significantly reduced when CHD4 was depleted. See, FIG. 49D. These DNA fiber phenotypes were also observed with a second shRNA reagent to CHD4. See, FIG. 50A, 50B and 51E. However, when CHD4 was depleted, replication restraint in response to stress was not re-established. Instead, replication tracks were significantly longer in CHD4-depleted PEO1 cells, compared to PEO1 control or C4-2 cells. See, FIG. 49B. Moreover, in agreement with increased replication track length, analysis of global cellular replication by EdU incorporation demonstrated that CHD4 depleted PEO1 cells also had a greater number of EdU positive cells as compared to control PEO1 or C4-2 cells in response to HU. Instead, EdU positive cells were similar in unchallenged controls. See, FIGS. 49C and FIGS. 50A-50E. Thus, ssDNA gaps and chemosensitivity were suppressed by CHD4 depletion, but replication fork restraint was not restored, indicating replication was further mis-regulated. See, FIG. 49E. Notably, in C4-2 cells the BRCA2 reversion mutation (Sakai et al., 2009) also suppresses ssDNA gaps. See, FIGS. 47B and 49B. These data suggest that chemoresistance may be achieved by either gap filling or restored fork slowing.

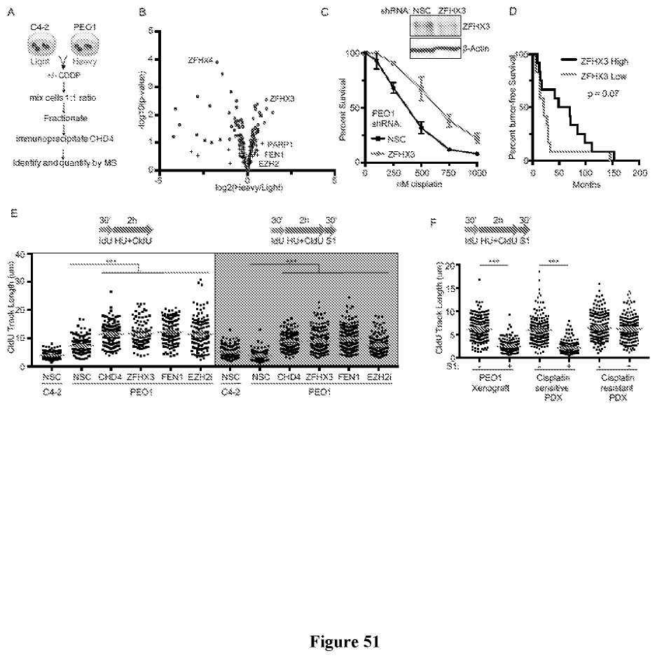

The data presented herein indicate that suppression of ssDNA replication gaps in BRCA deficient cancer could confer chemoresistance. To address this possibility, it was sought to identify additional genes similar to CHD4 that confer chemoresistance by gap suppression. Thus, quantitative mass spectrometry proteomics was perfomred comparing a CHD4-interactome between BRCA2 deficient vs proficient cells after cisplatin treatment. See, FIG. 51A. In BRCA2 deficient PEO1 cells, CHD4 interacted with several proteins known, when lost, to confer chemoresistance without restoring homologous recombination, including PARP1, EZH2, and FEN1. See, FIG. 51B. (Guillemette et al., 2015; Meghani et al., 2018; and Rondinelli et al., 2017). Also identified was the CHD4-interacting protein ZFHX3, whose depletion was found to enhance cisplatin resistance in PEO1 cells. See, FIG. 51C. (Chudnovsky et al., 2014). Moreover, in BRCA2 deficient ovarian cancers, low mRNA levels of ZFHX3 predicted poor tumor-free survival, as previously found for CHD4, EZH2, and FEN1 See, FIG. 51D. (Guillemette et al., 2015; Meghani et al., 2018; Rondinelli et al., 2017).

It was also observed that depletion of ZFHX3 or FEN1, or EZH2 inhibition, increased replication in the presence of HU, as found for CHD4 depletion. See, FIG. 51E. Moreover, in an S1 nuclease assay, inhibition of EZH2 or depletion of ZFHX3 or FEN1 also protected regions of nascent DNA from S1 nuclease degradation in BRCA2 deficient cells, similar to the depletion of CHD4. See, FIG. 51E and FIGS. 50A and 50F. Together, these findings suggest that loss of CHD4, EZH2, FEN1, and ZFHX3 suppresses ssDNA gaps to confer chemoresistance.

Next, ssDNA gaps were tested if they could predict chemosensitivity and resistance in BRCA patient tumor samples. A triple-negative breast cancer patient-derived xenograft (PNX0204), was utilized that harbored a hemizygous BRCA1 mutation (1105insTC). PNX0204 tumors were originally sensitive to cisplatin treatment. After several rounds of cisplatin treatment and serial passage in mice, resistant tumors developed. Isogenic sensitive and resistant tumors were then tested for S1 sensitivity, with PEO1 and MDA-MB-436 xenografts serving as controls. See, FIG. 51F and FIG. 50F. It was found that DNA fibers of cisplatin-sensitive PDX cells were degraded by S1 nuclease, but fibers of cisplatin-resistant isogenic PDX cells were not. Notably, resistant PDX suppressed gaps either by gap filling, or by restored fork slowing, indicating that loss of ssDNA gaps had occurred in BRCA patient tumors de novo and accurately predicted acquired cisplatin resistance See, FIG. 51F, and FIGS. 50F and 50H.

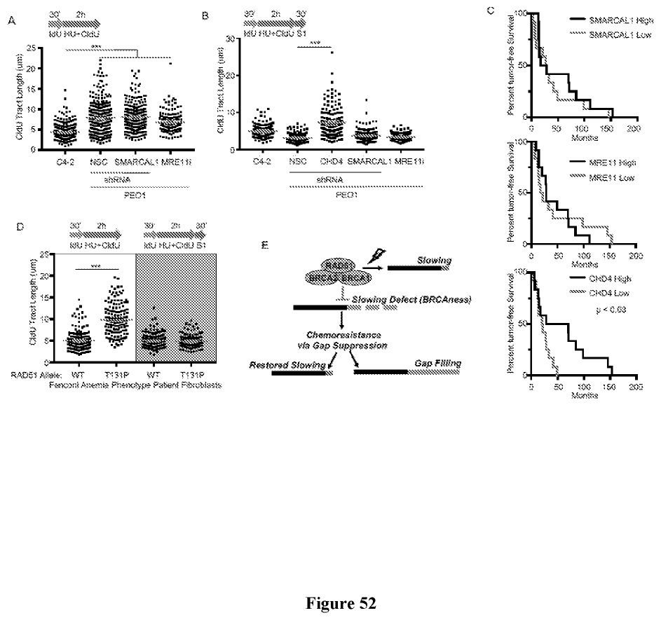

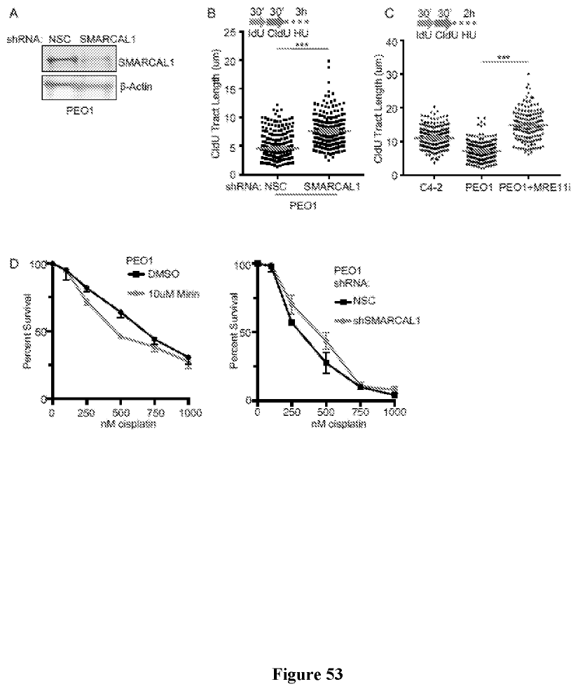

These findings suggest that ssDNA gaps underlie BRCAness and chemosensitivity, and that loss of replication fork protection or homologous recombination do not. If so, when ssDNA gaps are present, it should be possible to uncouple fork protection and homologous recombination from therapy response. To test this prediction, fork protection was restored by inhibition of MRE11 or depletion of SMARCAL1 in BRCA2-deficient PEO1 cells (Kolinjivadi et al., 2017; Schlacher et al., 2011; Taglialatela et al., 2017). Nevertheless, even though replication fork protection was restored, cisplatin resistance was not conferred and, as predicted herein, ssDNA gaps remained as demonstrated by the S1 nuclease degradation. See, FIGS. 52A, 52B and FIGS. 53A-C. Moreover, neither SMARCAL1 nor MRE11 was predictive of BRCA2 cancer patient response based on mRNA levels in the TCGA database suggesting that ssDNA gaps, but not replication fork protection, determines chemotherapy response and/or efficacy. See, FIG. 52C.

It was also considered a possibility that the presently disclosed ssDNA replication gap model could explain a discrepancy in the literature in which cells from a patient with Fanconi Anemia (FA) were sensitive to cisplatin and other genotoxic agents as expected, but were surprisingly found to be proficient in homologous recombination (Wang et al., 2015). Indeed, a wide-spread ssDNA gap induction was found in an S1 assay in these FA patient cells; specifically, S1 sensitivity was observed in an FA patient fibroblasts that maintain a RAD51 mutant (T131P) allele as compared to isogenic RAD51 wild type fibroblasts (CRISPR corrected after isolation from the patient). See, FIG. 52D. Together, these results suggest that ssDNA replication gaps have a superior predictive power as compared to either a fork protection or homologous recombination models of chemotherapeutic therapy response and/or efficacy, and suggests that ssDNA replication gaps represent a first-line genotoxic chemotherapy.

The above data showing that ssDNA gaps predict chemoresponse and/or chemotherapeutic efficacy has several implications. Namely, that replication ssDNA gaps may be resposnible for, at least, predicting chemotherapic response if not directly responsible for the toxic effects of most chemotherapeutics. Furthermore, it is shown that the failure to suppress ssDNA gaps, and not defects in homologous recombination or fork protection, that underlies the sensitivity of BRCA deficient cancer to chemo therapy. In support of this concept, when ssDNA gaps persist, it has been demonstrated that homologous recombination or fork protection proficient cells are nevertheless sensitive to cisplatin. Moreover, when ssDNA gaps are suppressed by loss of CHD4, FEN1, or EZH2, homologous recombination deficient BRCA2 mutant cells are resistant to cisplatin (Guillemette et al., 2015; Meghani et al., 2018; Rondinelli et al., 2017), suggesting chemoresponse is independent of homologous recombination.

Although ssDNA gaps are a common indicator of replication stress and result from loss of the BRCA-RAD51 pathway and can be detected in mouse mammary tissue deficient in BRCA1, they have been overlooked as the determinant of chemotherapeutic toxicity in favor of defects in homologous recombination and fork protection. (Hashimoto et al., 2010; Henry-Mowatt et al., 2003; Kolinjivadi et al., 2017; Sugimura et al., 2008; Wang et al., 2015; Xu et al., 2018; Zellweger et al., 2015). However, it is unclear how homologous recombination and fork protection participate in therapy response given that drugs used to treat BRCA deficient tumors do not initially cause DNA breaks or stall forks (Maya-Mendoza et al., 2018; Mutrej a et al., 2018). In contrast, replication ssDNA gaps arising due to loss of the BRCA-RAD51 pathway provides a logical explanation for the initiating lesion that is exacerbated by chemotherapies that further dysregulate replication.

When replication fails to be fully restrained due to loss of the BRCA-RAD51 pathway, the methods disclosed herein predict that replication ssDNA gaps derive from replication dysfunction rather than overactive nuclease activity (Kolinjivadi et al., 2017; Xu et al., 2018). Indeed, ssDNA length and S1 nuclease digestion were unaffected by MRE11 inhibition or depletion of the fork remodeler SMARCAL1, which generates the replication fork structure degraded by MRE11 in BRCA2 deficient cells(Kolinjivadi et al., 2017; Mijic et al., 2017; Taglialatela et al., 2017), suggesting that ssDNA gaps form in newly replicated DNA prior to remodeling or degradation of replication forks. CHD4 and its associated proteins could promote such replication gaps by mediating re-priming events downstream of the pre-existing replication fork. Several factors have been linked to re-priming including the polymerase primase, PrimPol (Guilliam and Doherty, 2017). Interestingly, the hyper-unrestrained replication achieved by CHD4 depletion in BRCA2 mutant cells correlates with a further reduction in the residual chromatin bound RAD51 (Guillemette et al., 2015). With RAD51 displaced, a separate pathway may have the opportunity to polymerize without gaps. Indeed, CHD4 depletion elevates the translesion synthesis (TLS) pathway(Guillemette et al., 2015) and TLS polymerases operating at the fork promote continuous replication without gaps(Cantor and Calvo, 2018). TLS may contribute to the mutation signature of BRCA2 mutant cancer cells(Nik-Zainal et al., 2016) and its further activation provides a mechanism for chemoresistance.

The data presented herein indicates that cancer cells with deficiency in a BRCA pathway can be effectively treated by chemotherapies that exacerbate replication ssDNA gaps. Moreover, preventing ssDNA gap suppression pathways improves the effectiveness of chemotherapy as well as potentially re-sensitizing chemoresistant disease to therapy. Based on these findings, ssDNA gaps could serve as biomarkers for BRCAness and that ssDNA gap induction could be involvedin chemotherapies that dysregulate replication.

### REFERENCES: SECTION VII

Cantor, S. B., and Calvo, J. A. (2018). Fork Protection and Therapy Resistance in Hereditary Breast Cancer. Cold Spring Harb Symp Quant Biol.
Carpenter, A. E., Jones, T. R., Lamprecht, M. R., Clarke, C., Kang, I. H., Friman, O., Guertin, D. A., Chang, J. H., Lindquist, R. A., Moffat, J., et al. (2006). CellProfiler: image analysis software for identifying and quantifying cell phenotypes. Genome Biol 7, R100.
Cerami, E., Gao, J., Dogrusoz, U., Gross, B. E., Sumer, S. O., Aksoy, B. A., Jacobsen, A., Byrne, C. J., Heuer, M. L., Larsson, E., et al. (2012). The cBio cancer genomics portal: an open platform for exploring multidimensional cancer genomics data. Cancer Discov 2, 401-404.
Chaudhuri, A. R., Callen, E., Ding, X., Gogola, E., Duarte, A. A., Lee, J. E., Wong, N., Lafarga, V., Calvo, J. A., Panzarino, N. J., et al. (2016). Replication fork stability confers chemoresistance in BRCA-deficient cells. Nature 535, 382-387.
Chudnovsky, Y., Kim, D., Zheng, S., Whyte, W. A., Bansal, M., Bray, M. A., Gopal, S., Theisen, M. A., Bilodeau, S., Thiru, P., et al. (2014). ZFHX4 interacts with the NuRD core member CHD4 and regulates the glioblastoma tumor-initiating cell state. Cell Rep 6, 313-324.
Cox, J., and Mann, M. (2008). MaxQuant enables high peptide identification rates, individualized p.p.b.-range mass accuracies and proteome-wide protein quantification. Nat Biotechnol 26, 1367-1372.
Gao, J., Aksoy, B. A., Dogrusoz, U., Dresdner, G., Gross, B., Sumer, S. O., Sun, Y., Jacobsen, A., Sinha, R., Larsson, E., et al. (2013). Integrative analysis of complex cancer genomics and clinical profiles using the cBioPortal. Sci Signal 6, pl1.

Guillemette, S., Serra, R. W., Peng, M., Hayes, J. A., Konstantinopoulos, P.A., Green, M. R., and Cantor, S. B. (2015). Resistance to therapy in BRCA2 mutant cells due to loss of the nucleosome remodeling factor CHD4. Genes Dev 29, 489-494.

Guilliam, T. A., and Doherty, A. J. (2017). PrimPol-Prime Time to Reprime. Genes (Basel) 8. Hashimoto, Y., Ray Chaudhuri, A., Lopes, M., and Costanzo, V. (2010). Rad51 protects nascent DNA from Mrell-dependent degradation and promotes continuous DNA synthesis. Nat Struct Mol Biol 17, 1305-1311.

Henry-Mowatt, J., Jackson, D., Masson, J. Y., Johnson, P. A., Clements, P. M., Benson, F. E., Thompson, L. H., Takeda, S., West, S. C., and Caldecott, K. W. (2003). XRCC3 and Rad51 modulate replication fork progression on damaged vertebrate chromosomes. Mol Cell 11, 1109-1117.

Kolinjivadi, A. M., Sannino, V., De Antoni, A., Zadorozhny, K., Kilkenny, M., Techer, H., Baldi, G., Shen, R., Ciccia, A., Pellegrini, L., et al. (2017). Smarcall-Mediated Fork Reversal Triggers Mrell-Dependent Degradation of Nascent DNA in the Absence of Brca2 and Stable Rad51 Nucleofilaments. Mol Cell 67, 867-881 e867.
Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079.
Maya-Mendoza, A., Moudry, P., Merchut-Maya, J. M., Lee, M., Strauss, R., and Bartek, J. (2018). High speed of fork progression induces DNA replication stress and genomic instability. Nature 559, 279-284.
McKenna, A., Hanna, M., Banks, E., Sivachenko, A., Cibulskis, K., Kernytsky, A., Garimella, K., Altshuler, D., Gabriel, S., Daly, M., et al. (2010). The Genome Analysis Toolkit: a MapReduce framework for analyzing next-generation DNA sequencing data. Genome Res 20, 1297-1303.
McLaren, W., Gil, L., Hunt, S. E., Riat, H. S., Ritchie, G. R., Thormann, A., Flicek, P., and Cunningham, F. (2016). The Ensembl Variant Effect Predictor. Genome Biol 17, 122.
Meghani, K., Fuchs, W., Detappe, A., Drane, P., Gogola, E., Rottenberg, S., Jonkers, J., Matulonis, U., Swisher, E. M., Konstantinopoulos, P. A., et al. (2018). Multifaceted Impact of MicroRNA 493-5p on Genome-Stabilizing Pathways Induces Platinum and PARP Inhibitor Resistance in BRCA2-Mutated Carcinomas. Cell Rep 23, 100-111.
Mijic, S., Zellweger, R., Chappidi, N., Berti, M., Jacobs, K., Mutreja, K., Ursich, S., Ray Chaudhuri, A., Nussenzweig, A., Janscak, P., et al. (2017). Replication fork reversal triggers fork degradation in BRCA2-defective cells. Nature communications 8, 859.
Mutreja, K., Krietsch, J., Hess, J., Ursich, S., Berti, M., Roessler, F. K., Zellweger, R., Patra, M., Gasser, G., and Lopes, M. (2018). ATR-Mediated Global Fork Slowing and Reversal Assist Fork Traverse and Prevent Chromosomal Breakage at DNA Interstrand Cross-Links. Cell Rep 24, 2629-2642 e2625.
Nik-Zainal, S., Davies, H., Staaf, J., Ramakrishna, M., Glodzik, D., Zou, X., Martincorena, I., Alexandrov, L. B., Martin, S., Wedge, D. C., et al. (2016). Landscape of somatic mutations in 560 breast cancer whole-genome sequences. Nature 534, 47-54.
O'Shaughnessy, A., and Hendrich, B. (2013). CHD4 in the DNA-damage response and cell cycle progression: not so NuRDy now. Biochemical Society transactions 41, 777-782.
Peng, M., Cong, K., Panzarino, N. J., Nayak, S., Calvo, J., Deng, B., Zhu, L. J., Morocz, M., Hegedus, L., Haracska, L., et al. (2018). Opposing Roles of FANCJ and HLTF Protect Forks and Restrain Replication during Stress. Cell Rep 24, 3251-3261.
Quinet, A., Carvajal-Maldonado, D., Lemacon, D., and Vindigni, A. (2017). DNA Fiber Analysis: Mind the Gap! Methods Enzymol 591, 55-82.
Ritchie, M. E., Phipson, B., Wu, D., Hu, Y., Law, C. W., Shi, W., and Smyth, G.K. (2015). limma powers differential expression analyses for RNA-sequencing and microarray studies. Nucleic Acids Res 43, e47.
Rondinelli, B., Gogola, E., Yucel, H., Duarte, A. A., van de Ven, M., van der Sluijs, R., Konstantinopoulos, P. A., Jonkers, J., Ceccaldi, R., Rottenberg, S., et al. (2017). EZH2 promotes degradation of stalled replication forks by recruiting MUS81 through histone H3 trimethylation. Nat Cell Biol 19, 1371-1378.
Sakai, W., Swisher, E. M., Jacquemont, C., Chandramohan, K. V., Couch, F. J., Langdon, S. P., Wurz, K., Higgins, J., Villegas, E., and Taniguchi, T. (2009). Functional restoration of BRCA2 protein by secondary BRCA2 mutations in BRCA2-mutated ovarian carcinoma. Cancer Res 69, 6381-6386.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682.
Schlacher, K., Christ, N., Siaud, N., Egashira, A., Wu, H., and Jasin, M. (2011). Double-strand break repair-independent role for BRCA2 in blocking stalled replication fork degradation by MRE11. Cell 145, 529-542.
Sugimura, K., Takebayashi, S., Taguchi, H., Takeda, S., and Okumura, K. (2008). PARP-1 ensures regulation of replication fork progression by homologous recombination on damaged DNA. J Cell Biol 183, 1203-1212.
Taglialatela, A., Alvarez, S., Leuzzi, G., Sannino, V., Ranjha, L., Huang, J. W., Madubata, C., Anand, R., Levy, B., Rabadan, R., et al. (2017). Restoration of Replication Fork Stability in BRCA1- and BRCA2-Deficient Cells by Inactivation of SNF2-Family Fork Remodelers. Mol Cell 68, 414-430 e418.
Wang, A. T., Kim, T., Wagner, J. E., Conti, B. A., Lach, F. P., Huang, A. L., Molina, H., Sanborn, E. M., Zierhut, H., Comes, B. K., et al. (2015). A Dominant Mutation in Human RAD51 Reveals Its Function in DNA Interstrand Crosslink Repair Independent of Homologous Recombination. Mol Cell 59, 478-490.
Xu, X., Chen, E., Mo, L., Zhang, L., Shao, F., Miao, K., Liu, J., Su, S. M., Valecha, M., In Chan, U., et al. (2018). BRCA1 represses DNA replication initiation through antagonizing estrogen signaling and maintains genome stability in parallel with WEE1-MCM2 signaling during pregnancy. Hum Mol Genet.
Zadorozhny, K., Sannino, V., Belan, O., Mlcouskova, J., Spirek, M., Costanzo, V., and Krejci, L. (2017). Fanconi-Anemia-Associated Mutations Destabilize RAD51 Filaments and Impair Replication Fork Protection. Cell Rep 21, 333-340.
Zellweger, R., Dalcher, D., Mutreja, K., Berti, M., Schmid, J.A., Herrador, R., Vindigni, A., and Lopes, M. (2015). Rad51-mediated replication fork reversal is a global response to genotoxic treatments in human cells. J Cell Biol 208, 563-579.

### Experimental

### EXAMPLE I

### Cell Lines

U2OS, PEO1, HeLa, MCF7, HCT15 and A549 cell lines were grown in DMEM supplemented with 10% fetal bovine serum and penicillin and streptomycin (100 U/ml each). MOLT4 and NCI-H522 cell lines were grown in RPMI supplemented with 10% fetal bovine serum and penicillin and streptomycin (100 U/ml each). HMEC, PD846F, and FA2819 cell lines were grown in DMEM supplemented with 15% fetal bovine serum and penicillin and streptomycin (100 U/ml each). FA-J cells (EUFA30-F) were immortalized with human telomerase reverse transcriptase (hTERT) 48 and cultured as previously described^{49,50}. Stable FA-J pOZ complemented cell lines were generated and selected as previously described (Peng et al., 2007). U2OS and HeLa FANCJ K/O CRISPR cell lines were generated as previously described⁵¹. Stable U2OS FANCJ K/O and PEO1 pLenti complemented cell lines were generated by blasticidin selection (5 μg/ml). Stable HeLa and U2OS shRNA knockdown cell lines were generated by puromycin selection (0.25 and 0.5m/m1 respectively).

PEO1, C4-2, VC-8, and MDA-MB-436 cell lines were cultured in DMEM+10% FBS+1% P/S. HCC1937 Deficient and HCC1937+WT BRCA1 were cultured in RPMI1640+L-Glutamine+10% FBS+1% P/S.

### EXAMPLE II

### Human Subjects

Malignant ovarian cancer cells were recovered from ascitic fluids from ovarian cancer patients by the University of Massachusetts Medical School (UMMS) Biorepository and Tissue Bank. Patient consent was obtained prior to specimen collection under an UMMS Institutional Review Board (IRB)-approved protocol (H4721). Malignant ovarian cancer cells were recovered from ascitic fluids by centrifugation at 200 g and cryopreserved in RPMI media supplemented with 10% FBS and 10% DMSO. Cells were slowly frozen at −80° C. in an isopropanol bath overnight and stored long term in the vapor phase of a liquid nitrogen freezer. The consent process included conditions for sharing de-identified samples and information with other investigators. No identifiable information will be shared at any time per Health Insurance Portability and Accountability Act guidelines.

### EXAMPLE III

### Plasmid and shRNA Constructs

The WT and S990A FANCJ pLentiviral vectors were a gift from J Chen⁵². The WT and S990A pO₂ vectors were generated as previously described⁴⁸. HeLa FANCJ K/O and U2OS cells were infected with pLK0.1 vector containing shRNAs against non-silencing control (NSC) or one of three shRNAs against p21/CDKN1A (A) (Target region—3′UTR -CGCTCTACATCTTCTGCCTTA), (B) (CDS-GAGCGATGGAACTTCGACTTT) and (C) (CDS-GTCACTGTCTTGTACCCTTGT). shRNAs were obtained from UMMS shRNA core facility.

### EXAMPLE IV

### Drugs and Reagents

Cisplatin (Sigma), was prepared as a 1 mM solution in saline per the manufacturer's instructions. MMC (Sigma), was prepared by dissolving 0.5 mg/mL in water. HU (Sigma), was prepared fresh in complete media prior to experiments per the manufacturer's instructions. The ATR inhibitor, VE-821 (Selleckchem), was prepared as a 15 mM solution in DMSO. 5-chloro-2′ (CldU) and 5-Iodo-2′ -deoxyuridine (IdU) were obtained from Sigma. Click-iT EdU Alexa Fluor 488 Imaging Kit was obtained from Invitrogen.

The following drugs were used in the course of this study: PARP inhibitor olaparib (AZD-2281, SelleckChem), cisplatin (Sigma-Aldrich), camptothecin (Sigma-Aldrich), ATR inhibitor (VE-821, SelleckChem). Reagents used including 5-chloro-2′ -deoxyuridine (CldU) and 5-Iodo-2′ -deoxyuridine (IdU) were obtained from Sigma-Aldrich. Concentration and duration of treatment are indicated in the corresponding figures and sections.

The MRE11 inhibitor mirin (Sigma) was prepared as a 50 mM solution in DMSO and used at 50 uM for DNA fiber analysis and was added during the indicated step per the figure diagrams(Schlacher et al., 2011). EZH2 was inhibited with 5uM GSK126 (Selleck) and was added during the CldU+HU step in S1 and slowing experiments(Rondinelli et al., 2017). 5-chloro-2′ -deoxyuridine (CldU) and 5-Iodo-2′ -deoxyuridine (IdU) were obtained from Sigma.

### EXAMPLE V

### Immunoblotting and Antibodies

Cells were harvested, lysed and processed for Western blot analysis as described previously using 150 mM NETN lysis buffer (20 mM Tris (pH 8.0), 150 mM NaCl, 1 mM EDTA, 0.5% NP-40, 1 mM phenylmethylsulfonyl fluoride, 10 mg/ml leupeptin, 10 mg/ml aprotinin). For cell fractionation, cytoplasmic and soluble nuclear fractions wee isolated with an NE-PER Kit (Thermo) according to the manufacturer's protocol; to isolate the chromatin fraction, the insoluble pellet was resuspended in RIPA buffer and sonicated in a BioRuptor according to the manufacturer's protocol (Medium power, 20 minutes, 30 seconds on, 30 seconds off at 4° C.). Proteins were separated using SDS—PAGE and electro-transferred to nitrocellulose membranes. Membranes were blocked in 5% not fat dry milk(NFDM) phos-phate-buffered saline (PBS)/Tween and incubated with primary Ab for overnight at 4° C.

Antibodies for Western blot analysis included anti-bactin (Sigma), anti-FANCJ (E67), anti-CHK1 (Cell signaling), anti-phospho CHK1[s317] (Cell Signaling), anti-RPA (Bethyl), anti-phospho RPA [s33] (Bethyl), anti-H2B (Cell Signaling), anti-p21 (BD Pharmingen), anti-RAD51 [H-92] (Santacruz), anti-REV1 [H-300] (Santacruz), anti-RAD18 (abcam), anti-MRE11 (Novus Biologicals) and anti-Pol H [B-7] (Santacruz). Membranes were washed, incubated with horseradish peroxidase-linked secondary Abs (Amersham) for 1 h at room temperature and detected by chemiluminescence (Amersham).

### EXAMPLE VI

### Viability Assays

Cells were seeded onto 96 well plates (500 cells per well, performed in triplicates for each experiment) and incubated overnight. Next day the cells were treated with increasing dose of MMC for 1 hr in serum-free media or cisplatin or TLSi and maintained in complete media for 5 days. Percent survival was measured using CellTitre-Glo viability assay (Promega) photometrically in a microplate reader (Beckman Coulter — DTX 880 Multimode detector).

### EXAMPLE VII

### Immunofluorescence

Immunofluorescence was performed as described previously²⁷. Cells were grown on coverslips in 10 μM BrdU for 48 h before the treatment with drugs. Cells were then treated with the aforementioned drugs for 2 h. After treatment, cells were washed with PBS and pre-extracted (0.5% Triton X-100 made in phosphate-buffered saline (PBS) on ice. Cells were then fixed using 4% Formalin for 15 min at RT. Fixed cells were then incubated with primary Abs against BrdU (Abcam) at 37° C. for 1 h. Cells were washed and incubated with secondary Abs for 1 h at room temperature. After washing, cover slips were mounted onto glass slides using Vectashield mounting medium containing DAPI (Vector Laboratories). For EdU labeling, staining was carried out with Click-iT EdU imaging kit (Invitrogen) according to the manufacturer's instructions.

### EXAMPLE VIII

### DNA Fiber Assays

To directly visualize replication fork dynamics, a single molecular DNA fiber analysis was established. In this assay, progressing replication forks in cells were labeled by sequential incorporation of two different nucleotide analogues, 5-Iodo-2′-deoxyuridine (IdU, 50 μM) and 5-Chloro-2′-deoxyuridine (CldU, 50 μM), into nascent DNA strands for the indicated time and conditions. After nucleotide analogues were incorporated in vivo, the cells were collected, washed, spotted (2.5 μl of 105 cells/ml PBS cell suspension) and lysed on positively charged microscope slides (Globe Scientific, #1358W) by 7.5 μl spreading buffer (0.5% SDS, 200 mM Tris-Hcl pH 7.4, 50 mM EDTA) for 8 min at room temperature. Individual DNA fibers were released and spread by tilting the slides at a 45 degree. After air dry, the fibers were fixed by 3:1 methanol: acetic acid at room temperature for 3 min. After air dry again, fibers were rehydrated in PBS, denatured with 2.5M HCl for 30 min, washed with PBS and blocked with blocking buffer (3% BSA and 0.1% Trition in PBS) for 1 hour. Next, slides were incubated for 2.5 h with primary Abs for (IdU: 1:100, mouse monoclonal anti-BrdU, Becton Dickinson 347580; CldU: 1:100, rat monoclonal anti-BrdU, Abcam 6326) diluted in blocking buffer, washed several times in PBS, and then incubated with secondary Abs (IdU: 1:200, goat antimouse, Alexa 488; CldU: 1:200, goat anti-rat, Alexa 594) in blocking buffer for 1 h. After washing and air dry, the slides were mounted with Prolong (Invitrogen, P36930). Finally, the visualization of green and/or red signals by fluorescence microscopy (Axioplan 2 imaging, Zeiss) will provide information about the active replication directionality at single molecular level.

### EXAMPLE IX

### S1 Nuclease Fiber Assay

Cells were exposed to 50 uM IdU to label replication forks, followed by 50 uM CldU with 0.5 mM HU for 1 h. Subsequently, cells were permeabilized with CSK buffer (100 mM NaCl, 10 mM MOPS, 3 mM MgCl2 pH 7.2, 300 mM sucrose, 0.5% Triton X-100) at room temperature for 8 minutes, followed by S1 nuclease (20 U/ml) in S1 buffer (30 mM Sodium Acetate pH 4.6, 10 mM Zinc Acetate, 5% Glycerol, 50 mM NaCl) for 30 minutes at 37C. Finally, cells were collected by scraping, pelleted, resuspended in 100-500 ul PBS; 2 ul of cell suspension was spotted on a positively charged slide and lysed and processed in accordance with Example VIII.

### EXAMPLE X

### Electron Microscopy

For the EM analysis of replication intermediates, 5-10×10⁶ U2OS cells treated with or without 4 mM HU were collected and genomic DNA was cross-linked by two rounds of incubation in 10 μg ml-1 4,5,8-trimethylpsoralen (Sigma-Aldrich) and 3 min of irradiation with 366 nm UV light on a precooled metal block10,26. Cells were lysed and genomic DNA was isolated from the nuclei by proteinase K (Roche) digestion and phenol-chloroform extraction. DNA was purified by isopropanol precipitation, digested with PvuII HF in the proper buffer for 3-5 h at 37° C. and replication intermediates were enriched on a benzoylated naphthoylated DEAE-cellulose (Sigma-Aldrich) column. EM samples were prepared by spreading the DNA on carbon-coated grids in the presence of benzyl-dimethyl-alkylammonium chloride and visualized by platinum rotary shadowing. Images were acquired on a transmission electron microscope (JEOL 1200 EX) with side-mounted camera (AMTXR41 supported by AMT software v601) and analyzed with ImageJ (National Institutes of Health). EM analysis allows distinguishing duplex DNA—which is expected to appear as a 10 nm thick fiber, after the platinum/carbon coating step necessary for EM visualization—from ssDNA, which has a reduced thickness of 5-7 nm. The criteria used for the unequivocal assignment of reversed forks include the presence of a rhomboid structure at the junction itself in order to provide a clear indication that the junction is opened up and that the four-way junction structure is not simply the result of the occasional crossing of two DNA molecules²⁵. In addition, the length of the two arms corresponding to the newly replicated duplex should be equal (b=c), whereas the length of the parental arm and the regressed arm can vary (a≠b=c≠d). Conversely, canonical Holliday junction structures will be characterized by arms of equal length two by two (a=b, c=d).

### EXAMPLE XI

### Colony Formation Assay

For colony formation assays either 500 or 1000 cells per well were seeded into 6 well plates and were treated continuously with or without TLSi. Once the colonies had developed, the cells were fixed with 90% methanol and then stained with 0.05% (w/v) crystal violet solution. Plates were then imaged using ChemiDocTM Touch Imaging System (BioRad).

### EXAMPLE XII

### Statistical Methods

Statistical differences in DNA fiber assay, S1 Nuclease assay, Immunofluorescence and the alkaline BrdU comet assay were determined using upaired t test or a two-tailed Mann-Whitney test. Statistical analysis was performed using Excel and GraphPad Prism (Version 7.0). In all cases, *p<0.05, **p<0.01, *p<0.001, and ****p<0.0001.

### EXAMPLE XIII

### Gene Editing

Human RPE1-hTERT cell lines were grown in DMEM+GlutaMAX-I (Gibco, 10569) supplemented with 10% FBS and 1% Pen Strep (100 U/ml). U2OS, 293T, PEO1 and C4-2 cell lines were grown in DMEM (Gibco, 11965) supplemented with 10% fetal bovine serum (FBS) and 1% Pen Strep (100 U/ml). T2, BRS, BR5-R1 cell lines were cultured in DMEM (CORNING cellgro, 15-017-CV) with 10% FBS, penicillin and streptomycin (100 U/ml each), and 1% L-glutamine41. The resistant cell line BR5-R1 was maintained in 1 □M olaparib. FA Patient fibroblasts (RA2630) were cultured in DMEM (Gibco, 11965) supplemented with 10% FBS, 1% Pen Strep (100 U/ml), 1% GlutaMAX-I, 1% MEM NEAA and 1% Sodium pyruvate45. All the cell lines were cultured at 37° C., 5% CO2. The generation of RPE1-hTERT TP53−/−BRCA1 K/O and BRCA1/53BP1 double K/O Cas9 cells were described elsewhere30. FANCJ gene knockout in RPE1-hTERT TP53−/−Cas9 cells was introduced by using two synthesized gRNAs (gRNA #1: GGGTCGAGGAAAGGTAACGG, gRNA #2: GGCAATCACCACACCCTTCA) and the protocol from IDT technology. For more details, see supplement methods. U2OS FANCJ K/O and 293T FANCJ K/O cells were generated and maintained as previously described 24.

### EXAMPLE XVI

### RNA Interference

U2OS cells were reverse transfected using RNAiMAX transfection reagent (Life Technologies 13778150) and siRNA targeting FANCJ/BRIP1 (QIAGEN SI03110723), BRCA1 (QIAGEN SI00299495), or scrambled negative control (ORIGENE SR30004) in 6-well plates for 48 h before super-resolution microscopy analysis. Stably transduced cells were generated by infection with pLK 0.1 vectors containing shRNAs against non-silencing control (NSC) or one of the shRNAs against corresponding genes: p21(CDKN1A) includes (A) 5′-TAAGGCAGAAGATGTAGAGCG-3′, (B) 5′-AAAGTCGAAGTTCCATCGCTC-3′; 53BP1(TP53BP1) includes (A) 5′-AAACCAGTAAGACCAAGTATC-3′, (B) 5′-AATCAATACTAATCACACTGG-3′; CHD4 includes 5′-AATTCATAGGATGTCAGCAGC-3′; RADX (CXorf57) includes 5′ ATTTCCGTGGAATACTTTCAG-3′. The sequence information was obtained from Dharmacon website (https://dharmacon.horizondiscovery.com), and the shRNAs were obtained from the University of Massachusetts Medical School (UMMS) shRNA core facility. Cells were selected by puromycin for 3-5 days before experiments were carried out.

### EXAMPLE XV

### Immunofluorescence for ssDNA

Cells were grown on coverslips in 10 μM CldU for 48 h before the indicated treatment in figures without CldU. After treatment, cells were washed with PBS and pre-extracted by 0.5% Triton X-100 made in phosphate-buffered saline (PBS) on ice. Cells were then fixed using 4% Formalin for 15 min at RT, and then permeabilized by 0.5% Triton X-100 in PBS again. Permeabilized cells were then incubated with primary antibodies against CldU (Abcam 6326) at 37° C. for 1 h. Cells were washed and incubated with secondary antibodies (Alexa Fluor 594) for 1 h at room temperature. After washing, cover slips were mounted onto glass slides using Vectashield mounting medium containing DAPI (Vector Laboratories). Images were collected by fluorescence microscopy (Axioplan 2 and Axio Observer, Zeiss) at a constant exposure time in each experiment. Representative images were processed by ImageJ software. Mean intensities of ssDNA in each nucleus were measured with Cell Profiler software version 3.1.5 from Broad Institute.

### EXAMPLE XVI

### Combined DNA Fiber Assay and S1 Nuclease Analysis

Cells were labeled by sequential incorporation of two different nucleotide analogs, IdU and CldU, into nascent DNA strands for the indicated time and conditions. After nucleotide analogs were incorporated in vivo, the cells were collected, washed, spotted, and lysed on positively charged microscope slides by 7.5 mL spreading buffer for 8 min at room temperature. For experiments with the ssDNA-specific endonuclease S1, after the CldU pulse, cells were treated with CSK100 buffer for 10 min at room temperature, then incubated with S1 nuclease buffer with or without 20 U/mL S1 nuclease (Invitrogen, 18001-016) for 30 min at 37 C. The cells were then scraped in PBS +0.1% BSA and centrifuged at 7,000 rpm for 5 min at 4 C. Cell pellets were resuspended at 1,500 cells/mL and lysed with lysis solution on slides. Individual DNA fibers were released and spread by tilting the slides at 45 degrees. After air-drying, fibers were fixed by 3:1 methanol/acetic acid at room temperature for 3 min. After air-drying again, fibers were rehydrated in PBS, denatured with 2.5 M HCl for 30 min, washed with PBS, and blocked with blocking buffer for 1 hr. Next, slides were incubated for 2.5 hr with primary antibodies for (IdU, Becton Dickinson 347580; CldU, Abcam 6326) diluted in blocking buffer, washed several times in PBS, and then incubated with secondary antibodies (IdU, goat anti-mouse, Alexa 488; CldU, goat anti-rat, Alexa Fluor 594) in blocking buffer for 1 hr. After washing and air-drying, slides were mounted with Prolong (Invitrogen, P36930). Finally, visualization of green and/or red signals by fluorescence microscopy (Axioplan 2 imaging, Zeiss) provided information about the active replication directionality at the single-molecule level.

### EXAMPLE XVII

### Viability Assays

Cells were seeded onto 96-well plates (500 cells per well, performed in triplicate for each experiment group) and incubated overnight. The next day, cells were treated with increasing doses of drugs indicated in corresponding figures and maintained in complete media for 5 to 7 days. Percentage survival was measured photometrically using a CellTiter-Glo 2.0 viability assay (Promega) in a microplate reader (Beckman Coulter DTX 880 Multimode Detector).

### EXAMPLE XVIII

### STORM Analysis

For super resolution imaging experiments, cells were trypsinized and seeded on glass coverslips in six-well plates in low density. siRNA transfection, drug treatment, and EdU incorporation were performed directly on cells on coverslips. We used an optimized pre-extraction and fixation protocol for our immunofluorescence experiments in order to clearly visualize chromatin-bound nuclear fraction of cells and to minimize nonspecific antibody labeling from the cytoplasm and non-chromatin bound proteins that could significantly increase noise for image analysis. Cells were permeabilized with 0.5% Triton X-100 in ice-cold CSK buffer (10 mM Hepes, 300 mM Sucrose, 100 mM NaCl, 3 mM MgCl2, and 0.5% Triton X-100, pH=7.4) in RT for 10 minutes and fixed with 4% paraformaldehyde (Electron Microscopy Sciences 15714) in RT for 30 minutes. Following fixation, cells were washed twice with PBS and blocked with blocking buffer (2% glycine, 2% BSA, 0.2% gelatin, and 50 mM NH4Cl in PBS). For nascent DNA detection, cells were pulse-labeled with 10 μM EdU (ThermoFisher A10044), a thymidine analogue, 15 minutes before permeabilization and fixation so that it would be incorporated into nascent DNA during replication in S-phase cells. After fixation, EdU was tagged with Alexa Fluor 647 picolyl azide through click reaction (Click-iT chemistry, ThermoFisher, C10640). The cells were blocked with blocking buffer at least overnight at 4° C. Before imaging, the samples were stained with primary antibodies against rb-RPA70 (Abcam ab79398), rb-MCM6 (conjugated to AF568, Abcam ab211916), and ms-PCNA (Santa cruz sc-56) in blocking buffer for 1 h at RT, then secondary antibodies (goat anti-mouse AF488, Invitrogen A11029; goat anti-rabbit AF 750, Invitrogen A21039) in blocking buffer for 30minutes at RT. For super resolution imaging and other related processes, see supplement methods.

### EXAMPLE XIX

### PDX Studies

PNX0204 was derived at Fox Chase Cancer Center under IRB and IACUC approved protocols. PDX tumors were grown in NOD.Cg-Prkdcscid Il2rgtm1Wjl/SzJ (NSG) mice. Resistant PDX tumors were obtained from mice after tumors progressed on serial treatments of olaparib. The tumors were harvested at approximately 500 mm3 and dissociated in 0.2% collagenase, 0.33 mg/ml dispase solution for 3 h at 37° C. The dissociated cells were maintained at 37° C. in RPMI1640 +10% FBS and used for DNA fiber assays within 24 h of tumor extrackion. DNA fiber assay and S1 nuclease analysis were performed as described above.

### EXAMPLE XX

### shRNA

HEK293T cells were used to package lentiviral particles with the pLKO.1 shRNA system as previously described(Guillemette et al., 2015). Briefly, HEK293 cells were transfected with 1:1:2 μg of packaging plasmids versus shRNA hairpins on the pLKO.1 vector using Effectene transfection reagent (Qiagen) 48 h prior to harvesting supernatants. Supernatants were filtered and added to recipient cell lines with 1 μg/mL polybrene. Cells infected with shRNA vectors were selected with puromycin. For shRNA-mediated silencing, the following hairpins from The RNAi Consortium were obtained from GE Dharmacon:

CHD4-61, TRCN0000021361:
5′-GCTGACACAGTTATTATCTAT-3′
CHD4-62, TRCN0000021362:
5′-GCTGACACAGTTATTATCTAT-3′
ZFHX3-58, TRCN0000013558:
5′-GCCAGGAAGAATTATGAGAAT-3′
FEN1-32, TRCN0000049732:
5′-GATGCCTCTATGAGCATTTAT-3′
SMARCAL1-69, TRCN0000083569:
5′-GCGGAACTCATTGCAGTGTTT-3′

### EXAMPLE XXI

### CellTiter-Glo 2.0 Toxicity Assays

Cells were plated at 500 cells per well in the center wells of a 96 well plate in 200 ul volume and allowed to adhere for 36 h. Subsequently, drugs were added in a 100 ul volume and the cells were incubated for 10 days. CellTiter-Glo 2.0 (Promega) was used to quantify cell number by ATP. To prevent evaporation, all blank wells in the 96 well plate were filled with media, and each plate was placed in a humid chamber with PBS.

### EXAMPLE XXII

### Cell Fractionation

To isolate cytoplasmic, nuclear, and chromatin fractions for western blot or mass spectrometry, cells were lysed with the NE-PER kit according to the manufacturer's instructions (Thermo Scientific). To isolate chromatin fractions, the insoluble pellet that remains from NE-PER lysis was resuspended in 60 ul of 2× loading buffer with DTT, heated at 70 C for 10 minutes, and sonicated in a BioRuptor (Diagenode) for 20 minutes on high, with a cycle of 30 seconds on and 30 seconds off.

### EXAMPLE XXIII

### Western Blot

All steps were performed according to the manufacturer's instructions (Thermo Scientific). The protein concentration of different cellular fractions was determined by BCA Assay. Samples were reduced with DTT in LDS loading buffer, and heated at 70 C for 10 minutes. 40 ug total protein was fully resolved on either a Tris-Acetate gel (for large proteins) or a Bis-Tris gel (for small proteins), and transferred to a nitrocellulose membrane. The nitrocellulose membrane was processed for near-infrared quantitative westerns according to the manufacturer's protocol (LiCor). The membrane was allowed to dry for 20 minutes, total protein was stained with the REVERT stain as a total protein loading control, followed by blocking with Odyssey blocking buffer, treated with primary antibody overnight, followed by near-infrared secondary antibody (800CW) at 1:5000 for 1 h. The membrane was allowed to dry prior to imaging on the LiCor Odyssey Imager. Primary antibodies used include anti-BRCA2 (Abcam ab123491, 1:1000); anti-CHD4 (Abcam ab54603 1:1000); anti-CHD4 (Abcam ab70469, for immunoprecipitation); anti-SMARCAL1 (Abam ab37003, 1:1000); anti-ZFHX3 (Lifespan Biosciences LS -C179898-100); anti-PCNA (Abcam ab29); anti-B-actin (Sigma A5441, 1:15,000).

### EXAMPLE XXIV

### Proteomics

For SILAC, PEO1 were dual labeled in SILAC media with dialyzed FBS (Thermo Scientific) with heavy lysine (K+8) and heavy arginine (R+10) from Cambridge Isotope Labs. PEO1 and C4-2 (unlabeled SILAC media) were treated with cisplatin, collected with trypsin, counted, mixed at a 1:1 ratio, and fractionated together in the same Eppendorf tube with the NE-PER kit as described. Cellular fractions were fully resolved on SDS-PAGE gels, fixed with Imperial Protein Coomassie Stain (Thermo Scientific), washed in water overnight, and cut into 13 molecular weight regions corresponding to the protein marker standard. Each region was reduced with DTT, alkylated with iodoacetamide, and digested with Trypsin Gold for Mass

Spectrometry with ProteaseMAX according to the manufacturer's instructions (Promega). Peptides were dried in a speedvac, resuspended in 6 ul buffer A (0.1% formic acid), and 2 ul tryptic digests were analyzed on the Thermo Q-Exactive mass spectrometer coupled to an EASY-nLC Ultra system (Thermo Fisher). Peptides were separated on reversed phase columns (12 cm×100 μm I.D), packed with Halo C18 (2.7 um particle size, 90 nm pore size, Michrom Bioresources) at a flow rate of 300 nl/min with a gradient of 0 to 40% acetonitrile (0.1% FA) over 55 min. Peptides were injected into the mass spectrometer via a nanospray ionization source at a spray voltage of 2.2 kV. The mass spectrometer was operated in a data-dependent fashion using a top-10 mode(Peng et al., 2018).

### EXAMPLE XXV

### Processing of Proteomics Data

Raw proteomics data were analyzed with MaxQuant software(Cox and Mann, 2008). We required a false discovery rate (FDR) of 0.01 for proteins and peptides and a minimum peptide length of 7 amino acids. MS/MS spectra were searched against the human proteome from UniProt. For the Andromeda search, we selected trypsin allowing for cleavage N-terminal to proline as the enzyme specificity. We selected cysteine carbamidomethylation as a fixed modification, and protein N-terminal acetylation and methionine oxidation were selected as variable modifications. Two missed tryptic cleavages were allowed. Initial mass deviation of precursor ion was up to 7 ppm, mass deviation for fragment ions was 0.5 Dalton. Protein identification required one unique peptide to the protein group. Known contaminants were removed from the analysis. To identify statistical significance, isotopic ratios of identified proteins from three biological replicates were analyzed using the limma statistical package (Ritchie et al., 2015). The isotopic ratio obtained from MaxQuant was subsequently converted to log 2 scale and plotted against the −log 10(p-value) for each gene in GraphPad Prism.

### EXAMPLE XXVI

### TCGA Database Analysis

The TCGA database was used to identify ovarian cancer patients with germline mutations in BRCA2, and subsequently tested for correlation between patient progression free survival and the mRNA expression of genes of interest. To obtain patient germline sequencing data, we applied for access to protected TCGA patient data through NIH. The germline BAM sequencing data for each patient at the BRCA2 locus was downloaded. The BAM Slicing tool option and the GDC API were used to automate the process. Sliced BAM files were sorted and indexed using SAMTOOLS(Li et al., 2009), and mutations were identified using the Genome Analysis Tool Kit (GATK), Broad Institute(McKenna et al., 2010). We followed the GATK best practices for germline mutation calling until the last step of the protocol, where we used hard filtering instead of variant quality score recalibration (VQSR) because sliced BAM files at a single locus are not compatible with VQSR. Briefly, we called germline variants using the HaplotypeCaller tool in GVCF mode, consolidated the GVCF files using the GenomicsDBlmport Tool, and called mutations using the GenotypeGVCFs tool. The data were hard filtered to isolate BRCA2 germline mutations, and we classified mutations using the Variant Effect Predictor (VEP) from Ensembl(McLaren et al., 2016). Finally, mutations were selected that are predicted to disable BRCA2, including premature stop codons, frameshift mutations, and deletions. A case list of patient barcodes was compiled harboring at least one of these BRCA2 disabling mutations, and cBioPortal(Cerami et al., 2012; Gao et al., 2013) was used to obtain the progression free survival data and mRNA expression data for our genes of interest. Patients with mRNA expression of the target gene over the median were classified as high expression; patients below were classified as low expression. The survival curve of the low and high expression groups was plotted in GraphPad Prism, and significance was determined using the Log-rank test.

1-8. (canceled)

9. A method, comprising:
a) providing;
i) a cancer cell replicating a deoxyribonucleic acid (DNA) sequence; and
ii) a probe configured to detect a single stranded DNA (ssDNA) gap within said DNA sequence;
b) scoring a quantity of ssDNA gaps within said DNA sequence with said probe;
c) predicting that said cancer cell is a chemosensistive cancer cell when said quantity of ssDNA gaps is above a threshold number; and
d) contacting said chemosensitive cancer cell with a chemotherapeutic agent, wherein said chemotherapeutic agent is efficacious against said at least one chemosensitive cancer cell.

10. The method of claim 9, wherein said contacting comprises administering said chemotherapeutic agent to a patient.

11. The method of claim 9, wherein said contacting comprises an in vitro cell culture.

12. The method of claim 9, wherein said cancer cell is a breast cancer cell.

13. The method of claim 10, wherein said patient is responsive to said chemotherapeutic agent.

14. The method of claim 9, wherein said threshold number is the quantity of ssDNA gaps in a non-cancer cell.

15. A method, comprising:
a) providing;
i) a cancer cell replicating a deoxyribonucleic acid (DNA) sequence; and
ii) a probe configured to detect a single stranded DNA (ssDNA) gap within said DNA sequence;
b) scoring a quantity of ssDNA gaps within said DNA sequence with said probe;
c) predicting that said cancer cell is a chemoresistant cancer cell when said quantity of ssDNA gaps is below a threshold number; and
d) contacting said chemoresistant cancer cell with a first compound to induce a single stranded DNA (ssDNA) gap within said DNA sequence or a second compound to inhibit ssDNA gap filling of said DNA sequence, wherein said chemoresistant cancer cell is converted into a chemosensitive cancer cell.

16. The method of claim 15, wherein said cancer cell is in a patient.

17. The method of claim 16, wherein said contacting comprises administering said first or second compound to said patient.

18. The method of claim 15, wherein said method further comprises treating said chemosensitive cancer cell with a chemotherapetic agent.

19. The method of claim 15, wherein said cancer cell is a breast cancer cell.

20. The method of claim 18, wherein said chemotherapeutic agent is efficacious against said chemosensitive cancer cell.

21. The method of claim 15, wherein said chemosensitive cell comprises a quantity of ssDNA gaps that is above a threshold number.

22. The method of claim 15, wherein said threshold number is the quantity of ssDNA gaps in a non-cancer cell.
